SEQUENCE LISTING (4 sequences)

SEQ ID NO: 1 - protein, 9 aa, Artificial Sequence
  Chemically Synthesized
QQGEFLNYD

SEQ ID NO: 2 - protein, 17 aa, Artificial Sequence
  Chemically Synthesized
SEGNRWSNSTKGLFQRA

SEQ ID NO: 3 - protein, 9 aa, Artificial Sequence
  Chemically Synthesized
HSEGLFQRA

SEQ ID NO: 4 - protein, 840 aa, Homo sapiens
MALPRCTWPNYVWRAVMACLVHRGLGAPLTLCMLGCLLQAGHVLSQKLDDVDPLVATNFGKIRGIKKELNNEILGPVIQFLGVPYAAPPTGERRFQPPEPPSPWSDIRNATQFAPVCPQNIIDGRLPEVMLPVWFTNNLDVVSSYVQDQSEDCLYLNIYVPTEDGPLTKKRDEATLNPPDTDIRDSGGPKPVMVYIHGGSYMEGTGNLYDGSVLASYGNVIVITVNYRLGVLGFLSTGDQAAKGNYGLLDLIQALRWTSENIGFFGGDPLRITVFGSGAGGSCVNLLTLSHYSEGNRWSNSTKGLFQRAIAQSGTALSSWAVSFQPAKYARMLATKVGCNVSDTVELVECLQKKPYKELVDQDIQPARYHIAFGPVIDGDVIPDDPQILMEQGEFLNYDIMLGVNQGEGLKFVENIVDSDDGISASDFDFAVSNFVDNLYGYPEGKDVLRETIKFMYTDWADRHNPETRRKTLLALFTDHQWVAPAVATADLHSNFGSPTYFYAFYHHCQTDQVPAWADAAHGDEVPYVLGIPMIGPTELFPCNFSKNDVMLSAVVMTYWTNFAKTGDPNQPVPQDTKFIHTKPNRFEEVAWTRYSQKDQLYLHIGLKPRVKEHYRANKVNLWLELVPHLHNLNDISQYTSTTTKVPSTDITFRPTRKNSVPVTSAFPTAKQDDPKQQPSPFSVDQRDYSTELSVTIAVGASLLFLNILAFAALYYKKDKRRHDVHRRCSPQRTTTNDLTHAQEEEIMSLQMKHTDLDHECESIHPHEVVLRTACPPDYTLAMRRSPDDVPLMTPNTITMIPNTIPGIQPLHTFNTFTGGQNNTLPHPHPHPHSHSTTRV

(19) United States
(12) Patent Application Publication
(10) Pub. No.: US 2023/0079439 A1
(43) Pub. Date: Mar. 16, 2023

(54) Compositions and Methods for Enhancing Beta Cell Maturation, Health and Function

(71) Applicants: THE REGENTS OF THE UNIVERSITY OF CALIFORNIA, Oakland (CA); BAR-IL-AN UNIVERSITY, Ramat Gan (IL)

(72) Inventors: Steven Chessler, San Diego, CA (US); Arie Lev Gruzman, Jerusalem (IL); Jean-Paul Lellouche, Ashdod (IL); Anna Munder, Rishon LeZion (IL)

(21) Appl. No.: 16/467,586

(22) PCT Filed: Dec. 8, 2017
(86) PCT No.: PCT/US17/65234

§ 371 Date: Jun. 7, 2019

Related U.S. Application Data
(60) Provisional application No. 62/432,212, filed on Dec. 9, 2016.

(51) Int. Cl. C12N 15/79; G01N 33/68; A61K 47/69

(52) U.S. Cl. CPC C12N 15/79; G01N 33/68; A61K 47/6923; A61K 47/6929; C12N 2015/8518

(57) ABSTRACT

The present invention provides compositions and methods for enhancing beta cell maturation, health and function. The invention may be used for increasing insulin secretion in a cell, promoting the formation of cell clusters, or reducing cell death. In some embodiments, the compositions and methods provide a treatment for diabetes. In some embodiments, the composition comprises an agent which increases a β-cell surface protein expression, activity or both.

### CROSS-REFERENCE TO RELATED APPLICATIONS

The present application claims priority to U.S. Provisional Application No. 62/432,212, filed Dec. 9, 2016, which is incorporated by reference herein in its entirety.

### STATEMENT REGARDING FEDERALLY SPONSORED RESEARCH OR DEVELOPMENT

This invention was made with government support under Grant No. DK080971 awarded by The National Institutes of Health. The government has certain rights in the invention.

### BACKGROUND OF THE INVENTION

The loss or decreased function of β-cells, and specifically, the reduction in their insulin secretion capacity and responsiveness to glucose, play an essential role in the development and progression of type 1 and 2 diabetes (T1DM and T2DM). T1DM pathogenesis is directly related to the lack of insulin production due to the absence of β-cells. Despite enormous efforts to develop novel types of anti-T1DM therapies, supplying lifelong insulin is still the only effective method for treating this disease. However, also in non-insulin-dependent diabetes mellitus (T2DM), especially in the late stages of the disease, the decreased ability of the pancreas to supply insulin (owing to β-cell damage and malfunction) significantly contributes to hyperglycemia and to the consequent development of severe diabetic complications. Current treatments of T2DM have focused on improving peripheral insulin sensitivity, the inhibition of gluconeogenesis, and the stimulation of insulin secretion. However, they do not target progressive β-cell loss directly. Moreover, this loss leads to the lack of optimal glycemic control in the majority of T2DM patients and it cannot be controlled with commonly used antihyperglycaemic medications and/or insulin injections.

Thus, promising directions in the development of novel anti-T2DM therapies involves the reversal of β-cell dysfunction induced by hyperglycemia and hyperlipidemia and protection of β-cells in order to increase or at least to preserve their mass. Mass preservation could be achieved through the protecting action of small organic compounds or larger biotherapeutic agents, whereas mass increase could be achieved by the production of new β-cells. Indeed, multiple efforts have been invested in this direction over the last decade using approaches such as stem cells, β-cell proliferation, non-pancreatic cell reprogramming, among others. Using this last approach, injured and non-functional β-cells of a patient are replaced by his own mature and functioning β-cells typically administered via transplantation. This strategy is suitable for treating both types of diabetes.

Intensive research is ongoing in order to elucidate the best methods for transforming human stem cells to fully differentiated β-cells for transplantation. Such a transformation requires a three-step differentiation process, from pluripotent stem cells to endoderm cells, from endoderm cells to pancreatic progenitors, and from pancreatic progenitors to mature β-cells. Currently these steps are hampered by low efficiency (in particular, for the second stage), expensive manufacturing costs, and great batch-to-batch variation. Moreover, even when successfully manufactured, the resulting β-cells often fail to function following transplantation. This is because in order for these cells to function properly, they need to form unique 3D cellular cluster structures (islets), which are not fully created in vitro prior to transplantation. Thus, tissue-engineering strategies may be used to provide the 3D organization of maturated β-cells required for in vivo functioning.

Such artificial in vitro self-organization of β-cells may mimic the pancreatic development and play a key role in normal insulin secretion and β-cell survival following transplantation. Consistent with the explanation, it has been shown that clusters of β-cells have better physiological parameters than do single dispersed cells. For example, such 3D cultured structures lead to β-cells with improved insulin secretion regulation in comparison with traditional two-dimensional (2D) monolayer cultures, and in addition, 3D organization of β-cells induces differentiation and the formation of islet-like structures that display greater similarities to pancreatic islets.

Although it is known that the cellular machinery of the β-cells is similar to that of neurons, there have been no systematic efforts to utilize this understanding to expedite the identification of factors that may enhance the functionality of β-cells. In particular, a tactic for examining the parallels between β-cell membranes and the pre-synaptic zones of neurons in order to identify molecules that may stimulate the formation of islet-like clustering and increased insulin secretion has not been reported to date.

Postsynaptic neuroligins (NLs), and their major binding partners, the neurexins (NXs), mediate interactions between neurons, and guide the differentiation, maturation, stabilization, and plasticity of both inhibitory and excitatory synapses. Furthermore, their abnormal functions were linked to several central nervous system disorders such as autism and schizophrenia. It was shown that clustered NLs induce the formation of presynaptic zones when brought into contact with axonal membranes, and, similarly, that NXs trigger postsynaptic density formation. In addition, in neurons, these complexes mediate the maturation of synapses with the concomitant secretion of neurotransmitters. Like neurons, β-cells must be organized in 3D structures in order to function physiologically. For example, glucose-stimulated insulin secretion (GSIS) is markedly decreased when β-cells are dispersed, but this important β-cell function returns to normal when the cells are allowed to re-aggregate.

Given the parallels between synapses and β-cells, it was suggested that specific transcellular NL and NX interactions, which are interrupted by β-cell dispersal, should be re-formed in order to simulate the NL-NX interactions that occur between β-cells in their native, 3D environment or form islet-like conglomerates of cells, which would exhibit normal β-cell functioning. Indeed, it was recently shown that clusters of NL-2 (a member of the NL family) enhanced insulin secretion and improved pancreatic β-cell function. Moreover, it was demonstrated that in NL-2-deficient mice, β-cells are smaller, fewer in number, and display reduced insulin content and altered levels of insulin secretion. These results clearly indicate that NL-2 influences β-cell function and suggests that mimicking the NL-2 activity might recreate the 3D islet environment through interactions of the NL-2 mimic with endogenous neurexin or lead to the formation of 3D islet-like structures in vitro, which may become useful in transplantation therapy.

Magnetically responsive maghemite (γ-Fe₂O₃)-based nanoscale particles are currently the subject of much interest due to several factors, among which are their potential use as contrast agents for in vivo Magnetic Resonance Imaging (MRI) and their well-known versatile surface chemical engineering. In a previous work, highly stable non-aggregated hydrophilic maghemite (γ-Fe₂O₃) NPs were synthesized using high-power ultrasonication of Massart magnetite (Fe₃O₄) NPs in the presence of a powerful mono electronic Ceric Ammonium Nitrate (CAN) oxidant. The resulting chemically modified positively charged NPs formed a stable aqueous colloid/ferrofluid owing to a unique ultra-sound-mediated process of NP surface doping by positive Ce^{3/4+} atoms/cations.

This nanofabrication methodology was successfully extended to similar Ytterbium (III) cation-doped maghemite NPs, using Yb(III) perchlorate [Yb(ClO₄)₃] instead of CAN doping of Yb⁺³ atoms/cations present on the NP surface, which strongly promoted high colloid stabilization against NP aggregation in aqueous media/dispersions. Remarkably, this lanthanide cation Lewis acid-acting shell (i) provides an effective mode for attaching any organic species onto the NP surface (coordinative mode of binding), as well as (ii) strongly affects the NP T₂* MRI relaxivity feature. In addition and similarly to the previously formed Ce^{+3/4} cation-doped γ-Fe₂O₃ NPs, these high-power ultrasonic Yb(III)-perchlorate-mediated reaction conditions led to the parallel chemical formation of an organic polyCOOH shell onto the NP surface, providing a 2^{nd} orthogonal potential mode for the covalent attachment of organic species (EDC.HCl activation/coupling).

There is thus a need in the art for improved compositions and methods for treating diabetes. The present invention addresses this unmet need in the art.

### SUMMARY OF THE INVENTION

In one aspect, the invention provides composition comprising an agent that increases a β-cell surface protein activity. In one embodiment, the β-cell surface protein is CADM1, CADM2, CADM3, CADM4, LRRTM1, LRRTM2, LRRTM3, LRRTM4, NEUREXIN-1, NEUREXIN-2, NEUREXIN-3, Slitrk1, Slitrk2, Slitrk3, Slitrk4, Slitrk5, Slitrk6, PTPRD, PTPRS, LAR, NL-1, NL-2, NL-3, CLSTN1, CLSTN2, CLSTN3, IL1RAPL1, IL1RAcP, IL1RAcPb, SALM3, or SALM5. In one embodiment, the β-cell surface protein is NL-2.

In one embodiment, the agent comprises a protein, wherein the protein binds neurexin isoforms and mimics neuroligin activity. In one embodiment, the agent comprises an isolated peptide. In one embodiment, the isolated peptide comprises a β-cell surface protein-derived peptide. In one embodiment, the isolated peptide comprises a NL-2-derived peptide. In one embodiment, the isolated peptide comprises an amino acid sequence selected from SEQ ID NOs: 1-3. In one embodiment, the isolated peptide comprises a dimer of two peptides comprising at least one amino acid sequence selected from SEQ ID NOs: 1-3.

In one embodiment, the dimer is conjugated to PEG-2000. the isolated peptide is conjugated to the surface of a delivery vehicle. In one embodiment, the delivery vehicle is a nanoparticle, a liposome, or a lipid nanoparticle.

In one embodiment, the delivery vehicle is a nanoparticle. In one embodiment, comprises a maghemite. In one embodiment, the nanoparticle further comprises an Ytterbium. In one embodiment, the nanoparticle is a Yb(III) cation doped-maghemite nanoparticle.

The invention also provides a cell engineered to secrete insulin. In one embodiment, the cell expresses a recombinant β-cell surface protein or a β-cell protein-derived peptide. In one embodiment, the β-cell surface protein is CADM1, CADM2, CADM3, CADM4, LRRTM1, LRRTM2, LRRTM3, LRRTM4, NEUREXIN-1, NEUREXIN-2, NEUREXIN-3, Slitrk1, Slitrk2, Slitrk3, Slitrk4, Slitrk5, Slitrk6, PTPRD, PTPRS, LAR, NL-1, NL-2, NL-3, CLSTN1, CLSTN2, CLSTN3, IL1RAPL1, IL1RAcP, IL1RAcPb, SALM3, or SALM5. In one embodiment, the β-cell surface protein is NL-2 or an NL-2 derived peptide. In one embodiment, the cell expresses a peptide having an amino acid sequence selected from the group consisting of SEQ ID NOs: 1-3.

The invention also provides a method for treating or preventing a condition associated with reduced insulin secretion in a subject in need thereof. In one embodiment, the method comprises administering to the subject an effective amount of a composition comprising an agent that increases a β-cell surface protein activity.

In one embodiment, the β-cell surface protein is CADM1, CADM2, CADM3, CADM4, LRRTM1, LRRTM2, LRRTM3, LRRTM4, NEUREXIN-1, NEUREXIN-2, NEUREXIN-3, Slitrk1, Slitrk2, Slitrk3, Slitrk4, Slitrk5, Slitrk6, PTPRD, PTPRS, LAR, NL-1, NL-2, NL-3, CLSTN1, CLSTN2, CLSTN3, IL1RAPL1, IL1RAcP, IL1RAcPb, SALM3, or SALM5. In one embodiment, the β-cell surface protein is NL-2.

In one embodiment, the agent comprises an isolated peptide. In one embodiment, the isolated peptide comprises a β-cell surface protein-derived peptide. In one embodiment, the β-cell surface protein-derived peptide is an NL-2 derived peptide. In one embodiment, the isolated peptide comprises an amino acid sequence selected from SEQ ID NOs: 1-3.

In one embodiment, the isolated peptide comprises a dimer of two peptides comprising at least one amino acid sequence selected from SEQ ID NOs: 1-3. In one embodiment, the dimer is conjugated to PEG-2000.

In one embodiment, the isolated peptide is conjugated to the surface of a nanoparticle. In one embodiment, the nanoparticle is a Yb(III) cation doped-maghemite nanoparticle.

In one embodiment, the condition is diabetes.

The invention also provides a method for treating or preventing a condition associated with reduced insulin secretion in a subject in need thereof. In one embodiment, the method comprises differentiating a stem cell into a mature β-cell by culturing the stem cell in the presence of a composition comprising an agent that increases a β-cell surface protein activity, thereby producing a cluster of mature β-cells; and transplanting the cluster of mature β-cells to the subject.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A and FIG. 1B, depicts the structure of the NX-1/NL-4 complex. FIG. 1A depicts the NX-1/NL-4 complex (PDB code 2WQZ). The residues responsible for the hydrogen bonds between NX-1 and NL-4 are shown in stick representation (Thr 235, Pro 106, Ser 107, Arg 109 of NX-1, and residues Glu 361 and Asn 364 of NL-4. FIG. 1B depicts the coordination of calcium ion by residues Asp 137, Asn 238, Val 154, and Ile 236 of NX-1 and residues Gln 359 and Gly 360 of NL-4.

FIG. 2, comprising FIG. 2A through FIG. 2C, depicts molecular Dynamics (MD)-based analysis of the HSA-28/NX-1 interactions. FIG. 2A depicts the role of the calcium ion and of hydrogen bonds in maintaining the interaction between HSA-28 and NX-1. Snapshot from the MD shows the interactions between the peptide, the calcium, and NX-1 that are depicted in blue. Hydrogen bonds are shown in black. FIG. 2B depicts a 2D diagram showing the interaction between HSA-28 and NX-1. FIG. 2C depicts the distance between HSA-28 (Gln 359 and Gly 360) and the calcium ion as a function of simulation times in both 2 MD simulations. The distance has converged after ˜5000 ps, demonstrating a stable interaction between the two partners.

FIG. 3, comprising FIG. 3A through FIG. 3D, depicts the evaluation of the biological activity of HSA-112 in INS-1E cells. FIG. 3A depicts a dose-response analysis of the effect of HSA-112 on the rate of insulin secretion in INS-1E cells. The INS-1E cells were grown and treated with increasing doses of HSA-112 for 24 h, as described in Methods. Subsequently, cells were mixed with different concentrations of HSA-112, as indicated in the graph (red columns), PEG₂₀₀₀ (green columns), and free peptide (blue columns) and were seeded in 12-well plates. After 24 h the cells were taken to a GSIS in the presence of 2.5 mM (weak color bars) or 16.7 mM (strong color bars) glucose. FIG. 3B depicts the time-course analysis of the effect of HSA-112 on the rate of insulin secretion in INS-1E cells. The INS-1E cells were prepared for the experiment as described above with 50 μM of HSA-112, PEG₂₀₀₀, or free peptide. After incubation, as indicated in the graph, the cells were taken to a GSIS as described above. FIG. 3C depicts the cell protective effect of HSA-112 under oxidative stress conditions. Cells were prepared for the experiment as described in Methods. Trolox was used as a positive control at a concentration of 100 μM. The oxidative stress was induced in the tissue culture, as described in Methods. Standard MTT analysis (also described in Methods) was used for evaluating the effect of HSA-112 on cell viability. FIG. 3D depicts INS-1E cell proliferation. Cells were prepared for the experiment as described above. After 24 h the cells were colored by trypan blue and counted as described in Methods *p<0.05, n=6. MEAN±SE.

FIG. 4, comprising FIG. 4A through FIG. 4G, depicts the core characterization of the Yb(III)-γ-Fe₂O₃ NPs. FIG. 4A depicts TEM image, 50 nm scale bar. FIG. 4B depicts SAED pattern analysis: (#1 (plane 220), #2 (plane 311), #3 (plane 400), & #6 (plane 440) for crystallinity and inorganic phase confirmation. FIG. 4C depicts the size distribution by TEM (6.58 nm). FIG. 4D depicts XRD analysis. FIG. 4E depicts the XPS data and analysis of the C is area. FIG. 4F depicts the XPS data and analysis of the —Cl 2p area. FIG. 4G depicts the XPS data and analysis of the Cl 2s area. FIG. 4H depicts SQUID magnetization profile (Ms=70.2 emu/g) for super-paramagnetism feature checking.

FIG. 5, comprising FIG. 5A through FIG. 5C, depicts the Yb(III)-γ-Fe2O3 NP size distribution. FIG. 5A depicts NP size distribution by TEM (7.86±2.18 nm). FIG. 5B depicts a TEM image of HSA-28P. FIG. 5C depicts a TEM image of HSA-28P.

FIG. 6, comprising FIG. 6A and FIG. 6B, depicts the thermogravimetric analysis of HSA-28P. FIG. 6A depicts a TGA thermogram graph of Yb(III)-maghemite (black line), 100% peptide-Yb(III)-γ-Fe₂O₃ (red line), & 50% peptide-Yb(III)-γ-Fe₂O₃ NPs (blue line). FIG. 6B depicts weight loss derivative function graphs of Yb(III)-maghemite (black line), 100% peptide-Yb(III)-γ-Fe₂O₃ (red line), & 50% peptide-Yb(III)-γ-Fe₂O₃NPs (blue line).

FIG. 7, comprising FIG. 7A through FIG. 7G, depicts the evaluation of the biological activity of HSA-28P in INS-1E cells. FIG. 7A depicts dose-response analysis of the effect of HSA-28P on the rate of insulin secretion in INS-1E cells. The INS-1E cells were grown and trypsinized as described in Methods. Subsequently, cells were mixed with the indicated concentrations of HSA-28P (red columns), non-relevant peptide nanoparticles (NRPNP, green columns), and naked nanoparticles (NP, blue columns) and were seeded in 12-well plates. After 24 h the cells were taken to a GSIS in the presence of 2.5 mM (weak color bars) or 16.7 mM (strong color bars) glucose. FIG. 7B depicts time-course analysis of the effect of HSA-28P on the rate of insulin secretion in INS-1E cells. The INS-1E cells were prepared for the experiment as described above with 1.34 μM of both HSA-28P or NRPNP, and 0.76 μg/ml of NP. After incubation for the time indicated in the graph, the cells were taken to a GSIS as described in Methods. FIG. 7C depicts the measurement of insulin content. The RIA-assay was performed for INS-1E lysates. FIG. 7D depicts the Effect of HSA-28P on the cells viability under oxidative stress conditions. INS-1E cells were incubated for 24 h with a medium supplemented with HSA-28P (3 μM), or HSA-28P1/2 (1.5 μM), or NPs covered by phantom random peptide (PPNP, 3 μM), or NP (0.76 μg/ml), or HSA-28 (3 μM) and trolox, as a positive antioxidant control (1 mM). After the incubation time, 50 mU/ml of glucose oxidase (GO) was added for an additional 1.15 h. Upon completion of the experiments, a standard MTT assay was conducted as described in Methods. Cell viability is presented as a percentage in comparison to non-treated cells. FIG. 7E depicts the effect of HSA-28P on the cell proliferation rate. Experiments were conducted on INS-1E cells that were seeded in 6-well plates. INS-1E cells were treated as described in Panel D. Cells were detached by trypsin and counted as described in Methods and then were visualized with a Cells Sense Live Imaging microscope. FIG. 7F depicts light microscope images of untreated cells. FIG. 7G depicts light microscope images of cells that were treated with HSA-28P. *p<0.05, n=3. MEAN±SE.

FIG. 8, comprising FIG. 8A through FIG. 8E, depicts HSA-28P cell binding. FIG. 8A depicts alive INS-1E cells were treated with “naked” NPs. NPs were labeled by FITC. The light microscope images were taken in different fields. Representative images are shown. FIG. 8B depicts alive INS-1E cells were treated with HSA-28P. NPs were labeled by FITC. The light microscope images were taken in different fields. Representative images are shown. FIG. 8C depicts Cells Sense Live Imaging microscope and fluorescent images treated with “naked” FITC-NPs. FIG. 8D depicts Cells Sense Live Imaging microscope and fluorescent images treated with HSA-28P. FIG. 8C depicts the quantitation results of the fluorescent green signal. The signal intensity was normalized by the number of cells. *p≤0.05, MEAN±SE.

FIG. 9, comprising FIG. 9A and FIG. 9B, depicts the cellular localization of HSA-28P. FIG. 9A depicts confocal microscopy images of INS-1E cells treated with FITC-labeled “naked” NPs (green) and then fixed. The obtained slides were stained with Alexa Fluor 633 Phalloidin for membrane (red) and with DAPI for nuclei (blue) Cells were then visualized with a Confocal-Zeiss microscope. FIG. 9B depicts confocal microscopy images of INS-1E cells treated with FITC-labeled HSA-28P NPs and then fixed. The obtained slides were stained with Alexa Fluor 633 Phalloidin for membrane (red) and with DAPI for nuclei (blue) Cells were then visualized with a Confocal-Zeiss microscope.

FIG. 10, comprising FIG. 10A through FIG. 10D, depicts the HSA-28P effect on C-peptide level. FIG. 10A depicts confocal microscopy images of INS-1E cells. FIG. 10B depicts confocal microscopy images of INS-1E cells treated with “naked” NPs. FIG. 10C depicts confocal microscopy images of INS-1E cells treated with HSA-28P. Cells were treated with antibody against C-peptide (green) according to the manufacturer's protocol and fixed. The obtained slides were stained with Alexa Fluor 633 Phalloidin for membrane (red) and with DAPI for nuclei (blue). FIG. 10D depicts quantitation results of the C-peptide signal. The signal intensity was normalized by the total intensity of the membrane signal. *p≤0.05, MEAN±SE.

FIG. 11, comprising FIG. 11A through FIG. 11D, depicts cell proliferation effect of HSA-28P does not induce glucagon synthesis. FIG. 11A depicts confocal microscopy images of INS-1E cells. FIG. 11B depicts confocal microscopy images of INS-1E cells treated with “naked” NPs. FIG. 11C depicts confocal microscopy images of INS-1E cells treated with HSA-28P. FIG. 11D depicts confocal microscopy images of INS-1E cells treated antibody against glucagon (green). The obtained slides were stained with Alexa Fluor 633 Phalloidin for membrane (red) and with DAPI for nuclei (blue). Cells were then visualized with a Confocal-Zeiss microscope. The signal intensity was normalized by the total intensity of the membrane signal. *p≤0.05, MEAN±SE.

FIG. 12, comprising FIG. 12A though FIG. 12C, depicts the effect of HSA-28P on insulin secretion in mouse islets. FIG. 12A depicts a dose-response analysis of the effect of HSA-28P on the rate of insulin secretion in isolated mouse islets. Islets were isolated and treated as described in Methods. Subsequently, HSA-28P was added to islets at the indicated concentrations. After 24 h the cells were taken to a GSIS in the presence of 2.5 mM (blue bars) or 16.7 mM (yellow bars) glucose. FIG. 12B depicts the effect of HSA-28P on the rate of insulin secretion compared to the control treatments in mouse islets. Briefly, 0.76 μg/ml of HSA-28P or HSA-28, or CNSP1, or CNSP2, or L-argenin (L-Arg) was added for 24 h. Thereafter, islets were taken to a GSIS as described in Methods. FIG. 12C depicts the measurement of insulin content. *p<0.05, n=3. MEAN±SE.

FIG. 13 depicts the chemical structure of the HSA-28 peptide derived from the NL-4/NX-1 complex.

FIG. 14 depicts the preparation of HSA-28P.

FIG. 15 depicts the calibration curve of HSA-28.

FIG. 16 depicts the evaluation of a possible HSA-28P stimulatory effect on the proliferation rate of the PC-3 and PC-12 cell lines. PC-3 cells or PC-12 cells were incubated for 24 h and the medium was supplemented with HSA-28P (2.76 μM), or NPs (0.76 μg/ml). After the incubation time, the cells were detached by trypsin, colored by trypan blue, and counted as described in Methods, n=6. MEAN±SE.

FIG. 17 depicts the preparation of a conjugate between PEG₂₀₀₀ and two molecules of the peptide (HSA-28). The commercially available PEG₂₀₀₀ was reacted with succinic anhydride in dry tetrahydrofurane as a solvent. After then a N-hydroxysuccinimide in pyridine was added to the reaction in precence of N,N-dicyclohexylcarbodiimide. The reaction was kept in room temperature under nitrogen atmosphere for 8 h. A solution of the peptide (HSA-28) in 15% bicarbonate in dioxane was added and the resulting mix was allowed to stir for additional 48 h. After filtration and HPLC purification, the substituted by the peptide in both ends of the PEG₂₀₀₀ (HSA-112) was obtained.

FIG. 18 depicts a model of the sites of β-cell-β-cell contact. Shown in red are the plasma membrane regions, pre-synaptic-like domains. Shown in yellow are the (postulated) post-synaptic-like domains. Show with a blue arrow is the transcellular binding and clustering of neuroligin-neurexin induces assembly of the insulin secretory machinery (including syntaxin-1 and CASK) around the neurexin cytoplasmic domain and of the gephyrin scaffold around the neuroligin cytoplasmic domain.

FIG. 19 depicts a 3D image showing punctate neurexin staining on the β-cell surface. Sections of rat pancreas were stained for insulin (green) and neurexin-1 (red) and imaged by confocal microscopy. The punctate nature of neurexin staining is visible in the 2-D image (inset) but is better seen in the 3-D image constructed from successive focal planes.

FIG. 20 depicts experimental results demonstrating soluble neuroligin-2 impairs glucose secretion by rat islets. Soluble neuroligin-2 extracellular domain was added to cultures of rat islets at the indicated concentrations. Insulin secretion was then measured (20 mM glucose). Neuroligin-2 exhibited half-maximal inhibition of insulin secretion at 9 nM. The soluble protein competes with endogenous, clustered neuroligin for binding to transcellular partners.

FIG. 21 depicts a schematic demonstrating examples of potential therapeutic agents as numbered on the figure above. The plasma membranes (blue) of two adjacent beta cells are depicted with proteins, including neuroligin (green), in the extracellular space between the two neighboring cells. 1, clustered recombinant NL-2 extracellular domain or a neuroligin-derived peptidomimetic agent to mimic the activity of endogenous neuroligin. 2, antibody-based reagent or other compound causing neurexin clustering (NX-1 is shown at a site of insulin secretion). 3, antibody-based reagent or other biologic causing clustering of endogenous NL-2. 4, a small molecule of biotherapeutic agent that binds a hypothetical, as of yet unidentified, high-affinity NL-2 binding partner.

FIG. 22, comprising FIG. 22A through FIG. 22C, depicts experimental results of incubation of INS1E cells with peptidomimetic agent. A peptidomimetic agent was designed to mimic neuroligin's binding site for neurexin. Monomers and dimers had no effect on insulin secretion. Multiple dimers were conjugated to PEG-2000 to simulate clustering (reagent HSA-112). Dimers were also clustered on nanoparticles (HSA-637). FIG. 22A depicts INS-1E cells were incubated for 5 h with HSA-112 (blue columns) or HSA-637 (purple) at different concentrations or with vehicle alone (black), washed, and then tested for insulin secretion at 3 mM glucose (striped columns) or at 20 mM glucose (solid columns). Treatment with 20 μM HSA-112 or 150 nM HSA-637 or greater significantly increased glucose-stimulated insulin secretion (*, p<0.05). FIG. 22B depicts a time course: insulin secretion was analyzed after the number of hours indicated following a 5 h incubation with 100 μM HSA-112 (blue), 500 nM HSA-637 (purple) or vehicle alone (black). Incubations were with 3 mM glucose (striped columns) or 20 mM glucose (solid columns). FIG. 22C depicts microscopy where INS-1E cells were incubated with HSA-637 conjugated to a fluorescent dye (Cy5, red) and then washed. Membranes were stained with Alexa Fluor 488 (green). For better visualization, microscopy was in hypotonic saline causing the INS-1E cells to swell and become more rounded. This demonstrates that coating the nanoparticle with HSA-28 caused binding to the INS-1E cell surface.

FIG. 23 depicts conjugation of the peptide to PAMAM dendrimer via maleimide moiety using iminothiolane reagent. HSA-28 was conducted to the free amine groups on the PAMAM according to Fmoc solid phase peptide synthesis protocol. The resulting compound (HSA-28D) compounds was dialyzed during 48 h using MIDI GeBaFlex-tube Dialysis Kit 3.5 kDa MWCO (Gene Bio-Application) and then HSA-28D was filtered into sterilized eppendorf tubes using syringe-driven filters, nylon membrane 0.22 μm. The rest of compounds were exposed to UV irradiation for 30 min.

FIG. 24 depicts insulin secretion by human islet cells after incubation with cross-linked, clustered rNL2ED-Fc. Indicated volume of solution containing cross-linked rNL2ED-Fc (90 μg/ml) or vehicle alone was added to equal numbers of dissociated human islet cells 4 hours prior to determination of GSIS. Stimulated insulin secretion is shown. The increase in secretion at basal glucose levels did not reach statistical significance (p<0.05). (n=6 wells, representative of 3 separate experiments).

FIG. 25 depicts insulin secretion by β cells cocultured with HEK293 cells expressing neuroligin-2 or neurexin-1α. MING β-cells were cultured with HEK293 cells previously transfected to express neuroligin-2 (NL-2), neurexin-1α (Nrxn) or a control protein (C). After 24 hours, GSIS was analyzed at basal (2.75 mM) and stimulating (18 mM) glucose concentrations. Insulin secretion was significantly increased (p<0.05) by both neuroligin and neurexin coculture.

FIG. 26 depicts EPAC-2 co-immunoprecipitates with neuroligin-2 from INS-1 cells. INS-1 cells were transfected to express FLAG-epitope-tagged neuroligin-2. After 48 h, neuroligin-2 was immunoprecipitated with an anti-FLAG antibody (NL2). Control immunoprecipitation (C) was with non-immune IgG. Immunoprecipitated proteins were analyzed by western blotting with an anti-FLAG and anti-EPAC-2 antibody.

FIG. 27, comprising depicts reduced insulin granule docking in neuroligin-2 global KO mice. EM was used to quantitate docked granules in control (WT) and mutant (KO) mice. Docked granules are those 100-200 nm from the plasma membrane. Consistent with the observed decrease in neurexin expression, there was decreased granule docking in the KO mice.

FIG. 28, comprising FIG. 28A and FIG. 28B, depicts an analysis of pancreas sections from newborn neuroligin-2 knockout mice. Pancreas sections were obtained from 3 to 4-day old control (white columns) and neuroligin-2 knockout (black columns) mice. FIG. 28A depicts, in adults, islet size was smaller in the mutant mice. FIG. 28B depicts the percentage of ki67-positive β-cells was greater in the mutant mice, indicative of in-creased proliferation (despite there being fewer β-cells per islet in both newborns and adults). Markers of apoptosis did not vary (not shown).

FIG. 29, comprising FIG. 29A through FIG. 29C, depicts an analysis of mice with β-cell specific Nrlgn2 KO induced at eight weeks of age. FIG. 29A depicts insulin tolerance testing revealed no difference in insulin sensitivity. FIG. 29B depicts IP glucose tolerance testing with 2 g/kg glucose shows impaired glucose tolerance in conditional KO mice vs littermate controls. AUC for glucose in KO mice is 28% greater than average AUC of controls (p<0.01). FIG. 29C depicts plasma insulin 15 minutes after glucose administration is decreased in KO mice. Studies were performed using male mice 18 days after tamoxifen treatment (n=7 to 9 in each group).

FIG. 30 depicts CADM expression in human islets and effect on insulin secretion in co-culture. qPCR analysis of expression of CADM isoforms in humans islets (black columns) relative to human brain (white, levels normalized to brain levels) (Left). A control protein (white columns) or CADM1 (black) was transfected into HEK293 cells. After 72 h, the transfected cells were co-cultured overnight with INS-1 cells and insulin secretion measured at low (3 mM) and high (20 mM) glucose concentrations (Right). Differences in secretion between control and CADM1 co-cultures were significant (P<0.05).

FIG. 31 depicts increased insulin secretion by beta cells treated with increasing amounts of lipid vesicles carrying recombinant neuroligin-2.

FIG. 32 depicts the stimulatory dose response and time course effect of HSA-28D on proliferation rate of INS-1E cells. INS-1E cells were seeded in 12 well plates. HSA-28D in various concentrations was added to cells and cells were visualized under Cells Sense Live Imaging microscope after 72 and 144 hours of incubation. N=9, representative pictures are shown.

FIG. 33 depicts the effect of HSA-28D on cell viability under oxidative and ER stress conditions. INS-1E cells were protected against oxidative stress during the incubation for 72 h with a medium supplemented with HSA-28D ([HSA-28]-0.003 mg/ml), in the presence of 50 mU/ml for last hour of glucose oxidase (GO, free radicals producer). Also, HSA-28D ([HSA]-0.003 mg/ml) protected INS-1E cells against ER stress which was induced by 0.113 μg/ml of thapsigargin (Tg) which was added for last 24 hours. A standard MTT assay was performed to estimate the cell viability in both experiments.

FIG. 34 depicts the positive effect of HSA-28D ([HSA]-0.003 mg/ml) on GSIS in high glucose concentration. In the presence of 3.3 mM (green bars) or 16.7 mM (blue bars) glucose a GSIS was conducted. The results are presented as a percentage relative to insulin secretion of control cells under conditions of low glucose. Results are normalized by total amount of protein and insulin content.

FIG. 35 depicts the positive effect of HSA-28D on the C-peptide intracellular accumulation. INS-1E cells were incubated for 72 h with HSA-28D ([HSA]-0.003 mg/ml). Cells were fixed with formaldehyde (4% in PBS), permeabilized with 0.1% Triton X-100 and exposed to anti C-peptide antibody (Abcam-ab14181) followed by secondary antibody (Abcam-ab150081) according to manufacturer's protocol. After that, the membranes of cells were exposed to Alexa Fluor 633 Phalloidin (Rhenium-A22284) and nuclei were stained with DAPI (Sigma Aldrich-F6057). Fluorescent signals were visualized with a Confocal-Zeiss microscope; the identical optical and imaging conditions were kept during experiments and visualization work. The experiment was run several times in triplicates with n=6 in each experiment.

FIG. 36 depicts experimental results demonstrating that Pdx1 (Pancreatic and duodenal homeobox 1), which is a transcription factor which necessary for pancreatic development and β-cell maturation, was also increased by the treatment of INS-1E cells by HSA-28D ([HSA]-0.003 mg/ml). INS-1E cells were incubated for 72 h with HSA-28D ([HSA]-0.003 mg/ml). Cells were fixed with formaldehyde (4% in PBS), permeabilized with 0.2% Triton X-100 and exposed to anti Pdx1 antibody (Abcam-ab47267) followed by secondary antibody (Abcam-ab150081) according to manufacturer's protocol. The membranes of cells were exposed to Alexa Fluor 633 Phalloidin (Rhenium-A22284) and nuclei were stained with DAPI (Sigma Aldrich-F6057). Fluorescent signals were visualized with a Confocal-Zeiss microscope; the identical optical and imaging conditions were kept during experiments and visualization work. The experiment was run several times using triplicates, n=6.

FIG. 37, comprising FIG. 37A and FIG. 37B, depicts the absence of the effect of HSA-28D ([HSA]-0.003 mg/ml) on the glucagon level in INS-1E cells FIG. 37A depicts results of experiments which were done on INS-1E cells that were seeded on coverslips in 6 well plates. INS-1E cells were incubated for 72 h with HSA-28D ([HSA]-0.003 mg/ml). Cells were fixed with formaldehyde (4% in PBS), permeabilized with 0.1% Triton X-100 and exposed to anti-Glucagon antibody (Abcam-ab8055) followed by secondary antibody (Abcam-ab150081) according to manufacturer's protocol. The membranes of cells were exposed to Alexa Fluor 633 Phalloidin (Rhenium-A22284) and nuclei were stained with DAPI (Sigma Aldrich-F6057). Fluorescent signals were visualized with a Confocal-Zeiss microscope; the identical optical and imaging conditions were kept during experiments and visualization work. The experiment was run several times using triplicates method. Representative pictures are shown. FIG. 37B depicts the glucagon positive control. The identical procedure described above was applied on a plate well coated by glucagon.

FIG. 38 depicts the in vivo effect of HSA-28D ([HSA]-0.003 mg/ml) on the blood glucose level in LDSTZ C57B black mice. HSA-28D (IP injection, 1.51 mg per mouse daily for 6 days) significantly decreased the level of blood glucose in mild diabetic mice, after 6 days of treatment.

FIG. 39 depicts increased insulin secretion by beta cells cultured in contact with COS cells transfected to express LAR (receptor-type tyrosine-protein phosphatase F, also known as leukocyte common antigen related protein). The black columns represent insulin secretion by beta cells cocultured with LAR-expressing cells at 2.7 mM glucose (low) or 17.7 mM glucose (high). White columns, negative control (COS cells were “mock” transfected). Green columns, COS cells were transfected to express NL-2. LAR, like NL-2 and the other proteins listed herein, is a protein expressed in both neurons and in beta cells and is present on both the beta cell surface and in the neuronal synaptic cleft.

FIG. 40, comprising FIG. 40A and FIG. 40B, depicts experimental results demonstrating that recombinant neuroligin-2 extracellular domain attached to an artificial lipid particle can improve pancreatic beta cell function. FIG. 40A depicts insulin normalized to cellular insulin content. FIG. 40B depicts absolute insulin secretion is shown.

FIG. 41, comprising FIG. 41A and FIG. 41B, depicts experimental results demonstrating that binding and clustering Nrxn1a with other agents activates the neuroligin-neurexin pathway. FIG. 41A depicts experimental results demonstrating that at low glucose (left), using an antibody to cluster neurexin (gray column) does not increase insulin secretion (blue column is non-transfected negative control). At high glucose levels (right three columns), the antibody increases insulin secretion (orange vs gray column). FIG. 41B depicts experimental results demonstrating that incubating with the antibody also increased the insulin content of the beta cells (blue column vs the other three). Increased insulin content was seen within 1 hour (red column) of antibody incubation.

### DETAILED DESCRIPTION

The present invention provides compositions and methods for inducing β-cell clusters, increasing insulin secretion of a cell, and/or enhancing cell survival. In some embodiments, the compositions of the invention may be used to treat conditions associated with impaired insulin secretion. For example, in some embodiments, the compositions of the invention may be used to treat or prevent diabetes. In one embodiment, the compositions may be used to increase insulin secretion by cultured, stem-cell-derived cells being differentiated into β-cell replacement cells for transplantation therapy.

In one embodiment, the composition comprises an agent, for example, an isolated peptide, isolated nucleic acid, small molecule, peptidomimetic, or the like, which increases β-cell surface protein expression, activity, or both. In one embodiment, the composition comprises a full-length β-cell surface protein. In one embodiment, the composition comprises a β-cell surface protein fragment or a β-cell surface protein-derived peptide. In some embodiments, the β-cell surface protein, protein fragment, or protein-derived peptide is, or is derived from, CADM1, CADM2, CADM3, CADM4, LRRTM1, LRRTM2, LRRTM3, LRRTM4, NEUREXIN-1, NEUREXIN-2, NEUREXIN-3, Slitrk1, Slitrk2, Slitrk3, Slitrk4, Slitrk5, Slitrk6, PTPD, PTPS, LAR, NL-1, NL-2, NL-3, CNSP-1, CNSP-2, CLSTN1, CLSTN2, CLSTN3, IL1RAPL1, IL1RAcP, IL1RAcPb, NGL1, NGL2, NGL3, SALM3, or SALM5.

In some embodiments, the composition comprises NL-2, an NL-2 fragment, or an NL-2-derived peptide. In some embodiments, the NL-2-derived peptide is conjugated to a nanoparticle. In one embodiment, the NL-2 derived peptide is conjugated to a Yb(III) cation doped-maghemite nanoparticle. In one embodiment, the NL-2 derived peptide is conjugated to PAMAM nanoparticles.

In one embodiment, the present invention provides methods for increasing insulin secretion in a subject in need thereof. For example, in one embodiment, the invention provides methods of treating a subject having, or at risk for developing, a condition associated with reduced insulin secretion. Exemplary conditions include, but are not limited to diabetes, metabolic syndrome, hyperuricemia, fatty liver, polycystic ovarian syndrome, and acanthosis nigricans. In one embodiment, the method comprises administering to the subject an effective amount of an agent that increases a β-cell surface protein expression, activity, or both.

In one embodiment, the method comprises contacting a cell with the composition in vitro or ex vivo to promote the differentiation of the cell into a β-cell. In one embodiment, the method comprises producing a cluster of β-cells. In one embodiment, the method comprises transplanting one or more clusters of β-cells in the subject.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type, might be abnormal for a different cell or tissue type.

The term “magnetic nanoparticle”, as used herein, refers to any nanoparticle having ferromagnetic and/or superparamagnetic behavior. Non-limiting suitable examples can include, Fe₂O₃, Fe₃O₄, Fe₂O₄, FeₓPt_{y}, CoₓPt_{y}, MnFeₓO_{y}, CoFeₓO_{y}, NiFeₓO_{y}, CuFeₓO_{y}, ZaFeₓO_{y}, and CdFeₓO_{y}, wherein x and y vary between 1 and 6, depending on the method of synthesis known in the art. In one embodiment, the magnetic nanoparticle comprises maghemite.

As used herein, “autologous” refers to a biological material derived from the same individual into whom the material will later be re-introduced.

As used herein, “allogenic” refers to a biological material derived from a genetically different individual of the same species as the individual into whom the material will be introduced.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process.

As used herein, “impaired insulin secretion” refers to an inability to secrete adequate insulin to maintain a normal blood glucose level. For such a patient, a “benefit” includes one or more of increased insulin production, and lowering or normalizing of blood sugar levels.

As used herein, the phrase “stem cells” refers both to the earliest renewable cell population responsible for generating cell mass in a tissue or body and the very early progenitor cells, which are somewhat more differentiated, yet are not committed and can readily revert to become a part of the earliest renewable cell population. The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The terms “precursor cell,” “progenitor cell,” and “stem cell” are used interchangeably in the art and as used herein refer either to a pluripotent or lineage-uncommitted progenitor cell, which is potentially capable of an unlimited number of mitotic divisions to either renew itself or to produce progeny cells which will differentiate into the desired cell type. In contrast to pluripotent stem cells, lineage-committed progenitor cells are generally considered to be incapable of giving rise to numerous cell types that phenotypically differ from each other. Instead, progenitor cells give rise to one or possibly two lineage-committed cell types.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

As used herein, the terms “subject” and “patient” are used interchangeably. As used herein, a subject is preferably a mammal such as a non-primate (e.g., cows, pigs, horses, cats, dogs, rats, etc.) and a primate (e.g., monkey and human), most preferably a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates, in part, to the unexpected finding that β-cell surface proteins, β-cell surface protein fragments derived from these proteins, or β-cell surface protein-derived peptides derived from these proteins, enhance β-cell function, such as glucose-stimulated insulin secretion, protection from stressful conditions, and the induction of the formation of β-cell clusters.

In one aspect, the present invention provides compositions for inducing β-cell clusters, increasing insulin secretion in a cell, and/or enhancing cell survival. In one embodiment, the composition comprises an agent that increases a β-cell surface protein activity or expression, or both. In one embodiment, the β-cell surface protein, protein fragment, or protein-derived peptide is, or is derived from a protein including, but not limited to, CADM1, CADM2, CADM3, CADM4, LRRTM1, LRRTM2, LRRTM3, LRRTM4, neurexin-1, neurexin-2, neurexin-3, Slitrk1, Slitrk2, Slitrk3, Slitrk4, Slitrk5, Slitrk6, PTPD, PTPS, LAR, NL-1, NL-2, NL-3, CNSP-1, CNSP-2, CLSTN1, CLSTN2, CLSTN3, IL1RAPL1, IL1RAcP, IL1RAcPb, SALM3, and SALM5. In some embodiments, the β-cell surface protein, protein fragment, or protein-derived peptide is, or is derived from NL-2.

In one embodiment, the composition comprises NL-2, an NL-2 fragment, or an NL-2-derived peptide. In one embodiment, the composition comprises an isolated peptide. In one embodiment, the isolated peptide is an NL-2 fragment or an NL-2 derived peptide. In one embodiment, the isolated peptide comprises an amino acid sequence of QQGEFLNYD (SEQ ID NO: 1), SEGNRWSNSTKGLFQRA (SEQ ID NO: 2) or HSEGLFQRA (SEQ ID NO: 3).

In some embodiments, the composition comprises a dimer of isolated peptides. For example, in one embodiment, the composition comprises a homodimer of SEQ ID NO:1. In one embodiment, the dimer is conjugated to PEG-2000.

In some embodiments, the composition comprises a nanoparticle. For example, in one embodiment, an isolated peptide of the invention is conjugated to the surface of a nanoparticle. In one embodiment, the isolated peptide is conjugated to the surface of a Yb(III) cation doped-maghemite nanoparticle. In one embodiment, the isolated peptide is conjugated to the surface of a PAMAM-based nanoparticle.

In some embodiments, the invention provides a cell engineered to secrete insulin. For example, the cell expresses a β-cell surface protein, β-cell surface protein fragment, or a β-cell protein-derived peptide.

In another aspect, the invention provides a method for increasing insulin secretion in a subject in need thereof. In one embodiment, the method treats or prevents a disease associated with reduced insulin secretion. In one embodiment, the method comprises administering to the subject a composition, which increases a β-cell surface protein expression, activity, or both. For example, in it is demonstrated herein that a β-cell surface protein-derived peptide, including a NL-2 derived peptide, increase insulin secretion from a β-cell.

In some embodiments, the method comprises transplanting one or more β-cells engineered to express a β-cell surface protein, a β-cell surface protein fragment, or a β-cell surface protein derived peptide. For example, in one embodiment, contacting a cell with the composition ex vivo to promote the differentiation of the cell into a β-cell. In one embodiment, the method comprises producing a cluster of β-cells. In one embodiment, the method comprises transplanting one or more clusters of β-cells in the subject.

### Compositions

In one aspect, the present invention provides compositions for inducing β-cell clusters. In order for β-cells to function properly, they need to form unique 3D cellular cluster structures (islets). Accordingly, the compositions may be used, for example, enhancing β-cell functions, including glucose-stimulated insulin secretion, and protecting β-cells under stress conditions. The compositions may also be used for treating diabetes. In some embodiments, the composition comprises an agent that increases the expression, activity, or both of a protein on the β-cell surface. In some embodiments, the composition comprises an agent that mimics the activity of a protein on the β-cell surface. In some embodiments, the protein on the β-cell includes, but is not limited to CADM1, CADM2, CADM3, CADM4, LRRTM1, LRRTM2, LRRTM3, LRRTM4, NEUREXIN-1, NEUREXIN-2, NEUREXIN-3, Slitrk1, Slitrk2, Slitrk3, Slitrk4, Slitrk5, Slitrk6, PTPRD, PTRPS, LAR, NL-1, NL-2, NL-3, NGL1, NGL2, NGL3, CNSP-1, CNSP-2, CLSTN1, CLSTN2, CLSTN3, IL1RAPL1, IL1RAcP, IL1RAcPb, SALM3, and SALM5. In some embodiments, the composition comprises an agent that binds to a protein on the β-cell surface. For example, in one embodiment, the composition comprises an agent that binds to neurexin-1.

In one embodiment, the composition comprises an agent that clusters endogenous neuroligin-2. For example, in one embodiment, the agent is an antibody, a peptide or a nanoparticle that clusters endogenous neuroligin-2. [[Inventors: Please add any additional agents that would cluster endogenous NL-2.]].

Exemplary agents, include, but are not limited to, isolated nucleic acids, vectors, isolated peptides, peptide mimetics, small molecules, and the like. In one embodiment, the agent is attached to the surface of a nanoparticle.

An agent that mimics or increases the activity of a protein on the β-cell surface is any agent that increases the normal endogenous activity associated with β-cell surface protein. In one embodiment, the agent modulates the level or activity of a β-cell surface protein by modulating the transcription, translation, splicing, degradation, enzymatic activity, binding activity, or combinations thereof, of the β-cell surface protein. In one embodiment, the agent increases the expression of a β-cell surface protein, thereby increasing the β-cell surface protein activity. In one embodiment, the agent increases the activity of endogenous a β-cell surface protein. In one embodiment, the agent has activity that mimics the normal endogenous activity associated with a β-cell surface protein. For example, in one embodiment, the composition of the present invention comprises isolated peptide fragments and β-cell surface protein-derived peptides that mimic endogenous β-cell surface protein activity. In some embodiments, composition of the present invention comprises isolated peptide fragments and neuroligin-2 (NL-2)-derived peptides that mimic endogenous NL-2 activity.

### Peptides

In one embodiment, the composition of the present invention comprises an isolated peptide comprising a β-cell surface protein, or biologically functional fragment thereof. The composition may comprise, for example, any isoform of a β-cell surface protein, including a β-cell surface protein from any organism. In one embodiment, the composition comprises a full-length β-cell surface protein. In one embodiment, the composition comprises a recombinant β-cell surface protein. In one embodiment, the composition comprises a fragment of a β-cell surface protein. In one embodiment, composition comprises a β-cell surface protein-derived peptide.

In one embodiment, the composition of the present invention comprises an isolated peptide comprising NL-2, or biologically functional fragment thereof. The composition may comprise, for example, any isoform of NL-2, including NL-2 from any organism. In one embodiment, the composition comprises full-length NL-2. In one embodiment, the composition comprises recombinant NL-2. In one embodiment, the composition comprises a fragment of NL-2. In one embodiment, the composition comprises an NL-2-derived peptide.

In one embodiment, the isolated peptide comprises human NL-2, or biologically functional fragment thereof. Exemplary human NL-2 amino acid sequences include, but are not limited to, amino acid sequences GenBank Accession No. AAM46111.1, GenBank Accession No. NP 065846.1, GenBank Accession No. Q8NFZ4.1, GenBank Accession No. NP 061850.2, GenBank Accession No. ALQ34110.1, GenBank Accession No. EAW90197.1, GenBank Accession No. EAW90195.1, and GenBank Accession No. EAW90196.1. However, the present invention is not limited to these particular sequences. Rather the present invention encompasses any NL-2 isoform from any source.

An exemplary human NL-2 amino acid sequence is:

(SEQ ID NO: 4)
MALPRCTWPNYVWRAVMACLVHRGLGAPLTLCMLGCLLQAGHVLSQKLDD
VDPLVATNFGKIRGIKKELNNEILGPVIQFLGVPYAAPPTGERRFQPPEP
PSPWSDIRNATQFAPVCPQNIIDGRLPEVMLPVWFTNNLDVVSSYVQDQS
EDCLYLNIYVPTEDGPLTKKRDEATLNPPDTDIRDSGGPKPVMVYIHGGS
YMEGTGNLYDGSVLASYGNVIVITVNYRLGVLGFLSTGDQAAKGNYGLLD
LIQALRWTSENIGFFGGDPLRITVFGSGAGGSCVNLLTLSHYSEGNRWSN
STKGLFQRAIAQSGTALSSWAVSFQPAKYARMLATKVGCNVSDTVELVEC
LQKKPYKELVDQDIQPARYHIAFGPVIDGDVIPDDPQILMEQGEFLNYDI
MLGVNQGEGLKFVENIVDSDDGISASDFDFAVSNFVDNLYGYPEGKDVLR
ETIKFMYTDWADRHNPETRRKTLLALFTDHQWVAPAVATADLHSNFGSPT
YFYAFYHHCQTDQVPAWADAAHGDEVPYVLGIPMIGPTELFPCNFSKNDV
MLSAVVMTYWTNFAKTGDPNQPVPQDTKFIHTKPNRFEEVAWTRYSQKDQ
LYLHIGLKPRVKEHYRANKVNLWLELVPHLHNLNDISQYTSTTTKVPSTD
ITFRPTRKNSVPVTSAFPTAKQDDPKQQPSPFSVDQRDYSTELSVTIAVG
ASLLFLNILAFAALYYKKDKRRHDVHRRCSPQRTTTNDLTHAQEEEIMSL
QMKHTDLDHECESIHPHEVVLRTACPPDYTLAMRRSPDDVPLMTPNTITM
IPNTIPGIQPLHTFNTFTGGQNNTLPHPHPHPHSHSTTRV

Exemplary NL-2 amino acid sequences from other sources include, but are not limited to NP 942562.2 (mouse), NP 446444.1 (rat) and NP 001285693.1 (Drosophila). Exemplary NL isoform sequences include NP 055747.1 (NL-1) and NP 851820.1 (NL-3).

In one embodiment, composition comprises an isolated NL-2-derived peptide. In one embodiment, the NL-2-derived peptide comprises a fragment of NL-2 that mimics the ability of NL-2 to induce β-cell cluster formation or the ability of NL-2 and the other NL isoforms to bind and cluster the protein neurexin on the β-cell surface. In one embodiment, the NL-2-derived peptide comprises a derivative of the NL-2 fragment. In one embodiment, the isolated peptide of the composition comprises an amino acid sequence selected from QQGEFLNYD (SEQ ID NO:1) and a dimer of QQGEFLNYD (SEQ ID NO:1).

In one embodiment, composition comprises an isolated CNSP-1-derived peptide. In one embodiment, the CNSP-1-derived peptide comprises a fragment of CNSP-1 that mimics the ability of CNSP-1 to induce β-cell cluster formation. In one embodiment, the CNSP-1-derived peptide comprises a derivative of the CNSP-1 fragment. In one embodiment, the isolated peptide of the composition comprises an amino acid sequence selected from SEGNRWSNSTKGLFQRA (SEQ ID NO:2) and a dimer of SEGNRWSNSTKGLFQRA (SEQ ID NO:2).

In one embodiment, composition comprises an isolated CNSP-2-derived peptide. In one embodiment, the CNSP-2-derived peptide comprises a fragment of CNSP-2 that mimics the ability of CNSP-2 to induce β-cell cluster formation. In one embodiment, the CNSP-2-derived peptide comprises a derivative of the CNSP-2 fragment. In one embodiment, the isolated peptide of the composition comprises an amino acid sequence selected from HSEGLFQRA (SEQ ID NO:3) and a dimer of HSEGLFQRA (SEQ ID NO:3).

The invention should also be construed to include any form of a peptide having substantial homology to a β cell surface protein, β cell surface protein fragment, or a β cell surface protein-derived peptide disclosed herein. Preferably, a peptide which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of a β cell surface protein or a β cell surface protein-derived peptide disclosed herein.

The invention should also be construed to include any form of a peptide having substantial homology to NL-2, NL-2 fragment, or a NL-2-derived peptide disclosed herein. Preferably, a peptide which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of NL-2 or a NL-2-derived peptide disclosed herein.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to stimulate the differentiation of a stem cell into the osteoblast lineage. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of a β-cell surface protein or a (3 cell surface protein-derived peptide, including but not limited to NL-2 or a NL-2 derived protein.

A peptide or protein of the invention may be phosphorylated using conventional methods such as the method described in Reedijk et al. (The EMBO Journal 11(4):1365, 1992).

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide, which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The invention also relates to peptides comprising a β-cell surface protein, such as NL-2, or a β cell surface protein-derived peptide, such as a NL-2-derived peptide, fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e., are heterologous).

In one embodiment, the targeting domain can be a membrane spanning domain, a membrane binding domain, or a sequence directing the protein to associate with for example vesicles or with the nucleus. In one embodiment, the targeting domain can target a peptide to a particular cell type or tissue. For example, the targeting domain can be a cell surface ligand or an antibody against cell surface antigens of a target tissue (e.g., bone, regenerating bone, degenerating bone, cartilage). A targeting domain may target the peptide of the invention to a cellular component.

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the β-cell surface peptide, β-cell surface protein fragment, or β-cell surface protein-derived peptide fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Nucleic Acids

In one embodiment, the present invention provides a composition comprising an isolated nucleic acid encoding a β-cell surface protein, β-cell surface protein fragment, a β-cell surface protein-derived peptide, or a biologically functional fragment thereof.

In one embodiment, the composition increases the expression of a biologically functional fragment of a β-cell surface protein. For example, in one embodiment, the composition comprises an isolated nucleic acid sequence encoding a biologically functional fragment of a β-cell surface protein. As would be understood in the art, a biologically functional fragment is a portion or portions of a full-length sequence that retain the biological function of the full-length sequence. Thus, a biologically functional fragment of the β-cell surface protein comprises a peptide that retains the function of full length the β-cell surface protein.

In one embodiment, the isolated nucleic acid sequence encodes NL-2 or a NL-2 fragment. In various embodiments, the isolated nucleic acid sequence encodes a NL-2-derived peptide comprising an amino acid sequence selected from SEQ ID NOs: QQGEFLNYD (SEQ ID NO:1) and a dimer of QQGEFLNYD (SEQ ID NO:1).

Further, the invention encompasses an isolated nucleic acid encoding a peptide having substantial homology to a β-cell surface protein, β-cell surface protein fragment, or a β-cell surface protein-derived peptide disclosed herein. In one embodiment, the isolated nucleic acid sequence encodes a β-cell surface protein, β-cell surface protein fragment, or a β-cell surface peptide mimetic having at least 75%, 80%, 85%, 90%, 95%, 96%, 97%, 98%, or 99% sequence homology with an amino acid sequence selected from SEQ NOs: 1-3.

The isolated nucleic acid sequence encoding a β-cell surface protein, β-cell surface protein fragment, or a β-cell surface protein-derived peptide can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding a β-cell surface protein, β-cell surface protein fragment, or a β-cell surface protein-derived peptide, or functional fragment thereof. In one embodiment, the composition comprises an isolated RNA molecule encoding a β-cell surface protein, β-cell surface protein fragment, or a β-cell surface protein-derived peptide, or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; 0- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In one embodiment, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In brief summary, the expression of natural or synthetic nucleic acids encoding a β-cell surface protein, β-cell surface protein fragment, or a β-cell surface protein-derived peptide is typically achieved by operably linking a nucleic acid encoding the β-cell surface protein, β-cell surface protein fragment, or a β-cell surface protein-derived peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In one embodiment, the vector also includes conventional control elements which are operably linked to the transgene in a manner, which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a β-cell surface protein, β-cell surface protein fragment, or a β-cell surface protein-derived peptide, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Particles

In one embodiment, the present invention provides a delivery vehicle comprising a β-cell surface protein, β-cell surface protein fragment, a β-cell surface protein-derived peptide, or a nucleic acid molecule encoding a β-cell surface protein, β-cell surface protein fragment, or a β-cell surface protein-derived peptide. Exemplary delivery vehicles include, but are not limited to, microspheres, microparticles, nanoparticles, polymerosomes, liposomes, dendrimers, micelles, and the like.

For example, in one embodiment, the delivery vehicle is decorated with a β-cell surface protein or a β-cell surface protein-derived peptide on the surface of the delivery vehicle. In one embodiment, the delivery vehicle comprises a targeting moiety that targets the delivery vehicle to a treatment site.

In one embodiment, NL-2, an NL-2 fragment, or an NL-2-derived peptide is conjugated to the surface of a nanoparticle. In some embodiments, the nanoparticle is a magnetic iron-based nanoparticle. In a preferred embodiment, the magnetic nanoparticle comprises maghemite. In one embodiment, the nanoparticle comprises an additional metal. In one embodiment, the additional metal is different from the metal which forms the magnetic nanoparticle. In one embodiment, the additional metal is Ytterbium. In one embodiment, the NL-2, NL-2 fragment, or NL-2-derived peptide is conjugated to the surface of the nanoparticle through the N-terminus of NL-2.

In one embodiment, the nanoparticle is a magnetic nanoparticle. For example, in one embodiment, the magnetic nanoparticle can comprise Fe₂O₃, Fe₃O₄, Fe₂O₄, FeₓPt_{y}, CoₓPt_{y}, MnFeₓO_{y}, CoFeₓO_{y}, NiFeₓO_{y}, CuFeₓO_{y}, ZaFeₓO_{y}, or CdFeₓO_{y}, wherein x and y vary between 1 and 6. In one embodiment, the nanoparticle is a Yb(III) cation-doped nanoparticle. In one embodiment, the magnetic nanoparticle is a maghemite (γ-Fe₂O₃) nanoparticle. In one embodiment, the nanoparticle is a Yb(III) cation-doped maghemite (γ-Fe₂O₃) nanoparticle.

In one embodiment, the nanoparticle is a Polyamidoamine (PAMAM) nanoparticle. PAMAM dendrimers are hyperbranched polymers with unparalleled molecular uniformity, narrow molecular weight distribution, defined size and shape characteristics and a multifunctional terminal surface. These nanoscale polymers consist of an ethylenediamine core, a repetitive branching amidoamine internal structure and a primary amine terminal surface. Dendrimers are “grown” off a central core in an iterative manufacturing process, with each subsequent step representing a new “generation” of dendrimer. Increasing generations (molecular weight) produce larger molecular diameters, twice the number of reactive surface sites, and approximately double the molecular weight of the preceding generation. The generation 5 PAMAM dendrimer was used which has a spheroidal, globular shape.

Liposomes, in one embodiment, increase intracellular stability, increase uptake efficiency and improve biological activity. In another embodiment, liposomes are hollow spherical vesicles composed of lipids arranged in a similar fashion as those lipids which make up the cell membrane. They have, in another embodiment, an internal aqueous space for entrapping water-soluble compounds and range in size from 0.05 to several microns in diameter. In another embodiment, liposomes can deliver peptides to cells in a biologically active form. In one embodiment, the liposome is a lipid nanoparticle.

In one embodiment, the composition comprises a lipid nanoparticle (LNP) and one or more nucleic acid molecules described herein. For example, in one embodiment, the composition comprises an LNP and one or more peptides.

The LNP may comprise any lipid capable of forming a particle to which the one or more nucleic acid molecules are attached, or in which the one or more nucleic acid molecules are encapsulated. The term “lipid” refers to a group of organic compounds that are esters of fatty acids and are characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

Exemplary liposomes, lipid particles and lipid nanoparticles are known in the art. See, e.g., U.S. patent application Ser. No. 14/964,381, which is incorporated by reference herein in its entirety.

### Scaffolds

The present invention provides a scaffold or substrate composition comprising a β-cell surface protein, β-cell surface protein fragment, a β-cell surface protein-derived peptide, a nucleic acid molecule encoding a β-cell surface protein, β-cell surface protein fragment, or β-cell surface protein-derived peptide, a cell producing a β-cell surface protein, β-cell surface protein fragment, or β-cell surface protein-derived peptide, or a combination thereof. For example, in one embodiment, a β-cell surface protein, β-cell surface protein fragment, a β-cell surface protein-derived peptide, a cell producing a β-cell surface protein, a β-cell surface protein fragment, or a β-cell surface protein-derived peptide, or a combination thereof within a scaffold. In another embodiment, a β-cell surface protein, a β-cell surface protein fragment, a β-cell surface protein-derived peptide, a cell producing a β-cell surface protein, β-cell surface protein fragment, or a β-cell surface protein-derived peptide, or a combination thereof is applied to the surface of a scaffold.

In one embodiment, the scaffold or substrate composition comprises NL-2, an NL-2 fragment, or an NL-2-derived peptide, a cell producing NL-2, an NL-2 fragment, or an NL-2-derived peptide, or a combination thereof.

The scaffold of the invention may be of any type known in the art. Non-limiting examples of such a scaffold includes a, hydrogel, electrospun scaffold, foam, mesh, sheet, patch, and sponge.

### Cells

In one embodiment, the present invention provides a cell or population of cells derived from the differentiation of precursor cell, including but not limited to, a stem cell, an endoderm cell, or a pancreatic progenitor cell. In one embodiment, the cell is a mature β-cell, derived from a stem cell, an endoderm cell, or a pancreatic progenitor cell. For example, it is described herein that β-cell surface proteins, fragments of β-cell surface proteins, and β-cell surface protein-derived peptides described herein provide the 3D clustering of mature β-cells derived from a stem cell, an endoderm cell, or a pancreatic progenitor cell. The stem cell from which the cell or cell population of the invention is derived, may be any type of stem cell, including, but not limited to, embryonic stem cell, adult stem cell, cord blood stem cell, cord tissue derived stem cell, induced pluripotent stem cell, and the like. In one embodiment, the stem cell is a pluripotent stem cell. In one embodiment, the stem cell is a human pluripotent stem cell.

In one embodiment, the mature β-cell of the invention is derived by contacting a precursor cell with an agent that mimics or simulates a β-cell surface protein expression, activity, or both. In some embodiments, the precursor cell is a pluripotent stem cell, an endoderm cell, or a pancreatic progenitor cell.

For example, in one embodiment, the mature β-cell is derived by culturing a stem cell in the presence of the β-cell surface protein, β-cell surface protein fragment, or β-cell surface protein-derived peptide. In one embodiment, the stem cell is cultured in a differentiation medium comprising a β-cell surface protein, β-cell surface protein fragment, or β-cell surface protein peptide mimetic. In one embodiment, the stem cell is cultured in the presence of a cell expressing and secreting a β-cell surface protein, β-cell surface protein fragment, or β-cell surface protein-derived peptide. For example, the stem cell may be cultured in the presence of a genetically modified cell, modified with an isolated nucleic acid to express and secrete a β-cell surface protein, β-cell surface protein fragment or a β-cell surface protein-derived peptide.

In another embodiment, the mature β-cell is derived by culturing a stem cell in the presence of NL-2, an NL-2 fragment, or an NL-2-derived peptide. In one embodiment, the stem cell is cultured in a differentiation medium comprising a NL-2 or NL-2 peptide mimetic. In one embodiment, the stem cell is cultured in the presence of a cell expressing and secreting NL-2, an NL-2 fragment, or an NL-2-derived peptide. For example, the stem cell may be cultured in the presence of a genetically modified cell, modified with an isolated nucleic acid to express and secrete NL-2, an NL-2 fragment, or an NL-2-derived peptide. In one embodiment, the NL-2, an NL-2 fragment, or an NL-2-derived peptide is conjugated to a nanoparticle. In one embodiment, the nanoparticle is a magnetic nanoparticle. In one embodiment, the nanoparticle is a Yb(III) cation doped-maghemite nanoparticle.

In one aspect, the differentiated mature β-cell may be used in the treatment of a condition associated with insufficient insulin secretion. In one embodiment, the differentiated mature β-cells may be used as research tools, used for example in drug discovery toxicity testing, disease pathology, and the like.

In one embodiment, the present invention provides a differentiation medium comprising an agent that mimics or increases a β-cell surface protein expression, activity, or both. For example, in one embodiment, the differentiation medium comprises a β-cell surface protein, β-cell surface protein fragment, or a β-cell surface protein-derived peptide. For example, in one embodiment, the differentiation medium comprises NL-2, an NL-2 fragment or an NL-2-derived peptide. The differentiation medium may comprise additional differentiation agents, including but not limited to Ca′, an epidermal growth factor (EGF), a platelet derived growth factor (PDGF), a keratinocyte growth factor (KGF), a transforming growth factor (TGF), cytokines such as an interleukin, an interferon, or tumor necrosis factor, retinoic acid, transferrin, hormones (e.g., androgen, estrogen, insulin, prolactin, triiodothyronine, hydrocortisone, or dexamethasone), sodium butyrate, TPA, DMSO, NMF (N-methyl formamide), DMF (dimethylformamide), or matrix elements such as collagen, laminin, heparan sulfate). The differentiation medium may also comprise one or more of pituitary extract (e.g. a bovine pituitary extract), steroid hormones (e.g. hydrocortisone, or a salt thereof such as the acetate), growth factors (e.g., epidermal growth factor, preferably human epidermal growth factor), catecholamines (e.g., epinephrine, either in racemic or enantiomeric form), iron-binding proteins (e.g., a transferrin), insulin, vitamins (e.g., retinoic acid), thyroid hormones (e.g., triiodothyronine), serum albumins (e.g., bovine or human serum albumin, including recombinant preparations), antibiotics (e.g., aminoglycoside antibiotics, such as gentamicin), and/or antifungals (e.g., amphotericin-B). In one embodiment, the differentiation medium comprises one or more agents typically found in osteogenic differentiation medium, including but not limited to dexamethasone, ascorbic acid, and β-glycerophosphate.

### Pharmaceutical Composition and Administration

Compositions comprising a β-cell surface protein, β-cell surface protein fragment, or a β-cell surface protein-derived peptide can be formulated and administered to a subject, as now described. For example, compositions of the invention for the treatment and/or prevention of a disease or disorder can be formulated and administered to a subject, as now described.

The invention encompasses the preparation and use of pharmaceutical compositions comprising a composition useful for the treatment or prevention of a disease or disorder, disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art. In various embodiments, the active ingredient is a β-cell surface protein, a β-cell surface protein-derived peptide, or a nanoparticle comprising a peptide of the invention, as elsewhere described herein.

As used herein, the term “pharmaceutically-acceptable carrier” means a chemical composition with which an appropriate β-cell surface protein, β-cell surface protein fragment or β-cell surface protein-derived peptide, may be combined and which, following the combination, can be used to administer the appropriate β-cell surface protein, β-cell surface protein fragment, or β-cell surface protein-derived peptide, to a subject.

The pharmaceutical compositions useful for practicing the invention may be administered to deliver a dose of between about 0.1 ng/kg/day and 100 mg/kg/day, or more.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate modulator thereof, according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions, which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, transdermal, subcutaneous, intramuscular, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

Pharmaceutically acceptable excipients used in the manufacture of pharmaceutical compositions include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Liquid formulations of a pharmaceutical composition of the invention may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers. The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to about 1000 mg per kilogram of body weight of the animal. The precise dosage administered will vary depending upon any number of factors, including, but not limited to, the type of animal and type of disease or disorder being treated, the age of the animal and the route of administration. Preferably, the dosage of the compound will vary from about 1 mg to about 100 mg per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease or disorder being treated, the type and age of the animal, etc.

### Methods of Producing Clustered β-Cells

In one aspect, the present invention provides a method of generating an clustered mature β-cells. For example, it is demonstrated herein that a β-cell surface protein, β-cell surface protein fragment, or β-cell surface protein-derived peptides induce clustering β-cell.

The production of a population of in vitro cultured cells of β-cell lineage derived from at least one stem cell includes culturing at least stem cell in vitro according to the method of the invention in order to produce β-cells which then form 3D clusters. The cells of the invention and cells derived therefrom can be derived from, inter alia, humans, primates, rodents and birds. Preferably, the cells of the invention are derived from mammals, especially mice, rats and humans. Stem cells from which the osteoblasts or osteoblast progenitor cells are derived may be either wild-type or genetically modified stem cells.

The cells of the present invention, whether grown in suspension or as adherent cell cultures, are grown in contact with culture media.

Culture media used in the present invention preferably comprise a basal medium, optionally supplemented with additional components.

Basal medium is a medium that supplies essential sources of carbon and/or vitamins and/or minerals for the cells. The basal medium is generally free of protein and incapable on its own of supporting self-renewal/symmetrical division of the cells.

Preferably, the suitable cell is isolated from a mammal, more preferably a primate and more preferably still, a human. The cells useful in the methods of the present invention are isolated using methods known in the art. Following isolation, the suitable cells are cultured in a culture medium. Media formulations that support the growth of cells include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5 A Medium, Medium M199 (M199E—with Earle's salt base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with nonessential amino acids), and the like.

In one embodiment, the cells are cultured in a differentiation medium, which include one or more agents that aid in the differentiation of a cell. For example, in one embodiment, the cells are cultured in an osteogenic differentiation medium, which comprises one or more agents that aid in the differentiation of the cell into the osteoblast lineage.

It is further recognized that additional components may be added to the culture medium. Such components include, but are not limited to, antibiotics, antimycotics, albumin, growth factors, amino acids, and other components known to the art for the culture of cells. Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml. However, the invention should in no way be construed to be limited to any one medium for culturing the cells of the invention. Rather, any media capable of supporting the cells of the invention in tissue culture may be used.

In one embodiment, the culture media comprises an agent that mimics or increases a β-cell surface protein expression, activity, or both. In some embodiments, the culture media comprises an agent that mimics or increases NL-2 expression, activity, or both. For example, the media may comprise an isolated β-cell surface protein-peptide, a β-cell surface protein-derived peptide, or derivatives and fragments thereof. In some embodiments, the media may comprise an NL-2 peptide, an NL-2-derived peptide, or derivatives and fragments thereof. In one embodiment, the method comprises culturing the stem cells in the presence of an agent that mimics or increases a β-cell surface protein expression, activity, or both only during final stages of stem cell differentiation. For example, in one embodiment, differentiation of the stem cell occurs over about 15-25 days in culture. In one embodiment, differentiation of the stem cell occurs over about 18-22 days in culture. Differentiation of the stem cell occurs over about 21 days in culture. For example, in one embodiment, the agent that mimics or increases a β-cell surface protein expression, activity, or both is administered only during the last 1-12 days of culture. In one embodiment, the agent that mimics or increases a β-cell surface protein expression, activity, or both is administered only during the last 5-10 days of culture. In one embodiment, the agent that mimics or increases a β-cell surface protein expression, activity, or both is administered only during the last 8 days of culture. In one embodiment, the agent that mimics or increases a β-cell surface protein expression, activity, or both is administered for 24-72 hours. In another embodiment, the agent that mimics or increases a β-cell surface protein expression, activity, or both is administered every 72 hours.

In one embodiment, culture media used in the invention do not contain any components which are undefined (e.g., serum and/or feeder cells), that is to say components whose content is unknown or which may contain undefined or varying factors that are unspecified. An advantage of using fully defined media, free of serum and free of serum extracts, is that efficient and consistent protocols for culture and subsequent manipulation of the cells of the invention and cells derived therefrom can be obtained.

Typical substrates for culture of the cells in all aspects of the invention are culture surfaces recognized in this field as useful for cell culture, and these include surfaces of plastics, metal, composites, though commonly a surface such as a plastic tissue culture plate, widely commercially available, is used. Such plates are often a few centimeters in diameter. For scale up, this type of plate can be used at much larger diameters and many repeat plate units used.

The culture surface may further comprise a cell adhesion protein, usually coated onto the surface. Receptors or other molecules present on the cells bind to the protein or other cell culture substrate and this promotes adhesion to the surface and promotes growth. In one embodiment, the cultures of the invention are preferably adherent cultures, i.e. the cells are attached to a substrate.

In one aspect, the cells from which β-cells are derived, are cultured in the presence of one or more additional cells that support the growth or differentiation of the cells. For example, the cells from which the β-cells are derived may be co-cultured with one or more cells genetically modified to express a β-cell surface protein, β-cell surface protein fragment, or β-cell surface protein-derived peptide.

### Treatment Methods

The present invention provides methods for the treatment or prevention of a disease or disorder associated with reduced insulin secretion in a subject in need thereof. Exemplary diseases or disorders treatable or preventable by way of the present invention includes, but is not limited to diabetes, prediabetes, metabolic syndrome, hyperuricemia, fatty liver, polycystic ovarian syndrome, acanthosis nigricans, pancreatic agenesis, pancreatitis and surgical pancreatectomy. In one embodiment, the disease or disorder is type 1 diabetes or type 2 diabetes.

In one embodiment, the methods comprises administering an effective amount of a composition described herein to a subject diagnosed with, suspected of having, or at risk for developing a disease or disorder associated with reduced insulin secretion. In one embodiment, the composition is administered systemically to the subject.

In one embodiment, the invention provides a method for β-cell health and function. In one embodiment, the invention provides a method for protecting β-cell from oxidative stress. In another embodiment, the invention provides a method for reducing β-cell death. In yet another embodiment, the invention provides a method for increasing β-cell insulin secretion.

In one embodiment, the method comprises administering an effective amount of a composition described herein to a cell in need thereof. In one embodiment, the composition is administered to a cell ex vivo. In another embodiment, the composition is administered to a cell in vivo. In one embodiment, the composition is administered to a cell in vitro.

The composition of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation or catheterization. Any administration may be a single application of a composition of invention or multiple applications. Administrations may be to single site or to more than one site in the individual to be treated. Multiple administrations may occur essentially at the same time or separated in time.

In one embodiment, the method comprises administering to a subject a nanoparticle comprising a β-cell surface protein, β-cell surface protein fragment, or a β-cell surface protein-derived peptide. In one embodiment, the method comprises administering to a subject a nanoparticle comprising NL-2, a NL-2 fragment or a NL-2 derived peptide. In one embodiment, the method comprises administering to a subject a nanoparticle comprising a peptide comprising SEQ ID NO:1. In one embodiment, the peptide is conjugated to the surface of the nanoparticle. In one embodiment, the nanoparticle is a Yb(III) cation-doped maghemite (γ-Fe₂O₃) nanoparticle. In one embodiment, the nanoparticle is a PAMAM nanoparticle.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the disease or disorder of the subject, and the type and severity of the subject's disease, although appropriate dosages may be determined by clinical trials.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are preferably administered by i.v. injection.

In one embodiment, the invention provides a method of treating a disease or disorder associated with reduced insulin secretion in a subject comprising transplanting a population of differentiated stem cells, or progeny thereof of the invention into the mammal. In one embodiment, the method comprises transplanting to a treatment site a population of differentiated stem cells, or progeny thereof, at least 95% of which have mature β-cell phenotype. In one embodiment, the mature β-cell is a cluster of mature of β-cells. In one embodiment, the mature β-cell is capable of secreting insulin. The population of cells is prepared in accordance with a method described herein, and is effective to repair at least a portion of the injured or diseased bone. In some embodiments, at least one differentiated stem cell, or progeny thereof, comprises a therapeutic transgene operably linked to a cell-specific promoter, wherein the transgene encodes a therapeutic gene product.

In some embodiments, a population of cells is transplanted directly to the pancreas. In some embodiments, transplanting the population of cells comprises administering a substrate or scaffold comprising the cells onto or into the pancreas. In one embodiment, the population of differentiated stem cells, or progeny thereof of the invention is at least 95%, preferably at least 96%, preferably at least 97%, more preferably at least 98%, more preferably at least 99% of which exhibit mature β-cell phenotype, wherein the population of cells is prepared in accordance with the methods of the invention, and is effective to secrete insulin.

Methods of treatment of the diseases encompassed by the invention can comprise the transplantation of single cells, cell lines, compositions, or cell populations of the invention into a subject in need thereof. In one embodiment, the subject is a human.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, are not to be construed as limiting in any way the remainder of the disclosure.

### Example 1: Mimicking Neuroligin-2 Functions in β-Cells by Functionalized Nanoparticles as a Novel Approach for Antidiabetic Therapy

The data presented herein, demonstrates that the NL-2-derived peptide conjugates onto innovative functional maghemite (γ-Fe₂O₃)-based nanoscale composite particles that enhance β-cell functions in terms of glucose-stimulated insulin secretion, and protect them under stress conditions. Recruiting the β-cells' “neuron-like” secretory machinery as a target for treatment use has never been reported before.

It is hypothesized herein that clusters of NL-2 might be introduced into the diabetic pancreas locally or that β-cell precursor cells might be differentiated with clusters of NL-2 to functionalize and protect β-cells inside the body. However, using full-length NL-2 is hampered by its rapid biodegradation and the potential induction of immunogenic responses. With this in mind, a short NL-2 mimetic peptide (HSA-28: QQGEFLNYD (SEQ ID NO:1)) derived from the NL-4/NX-1 crystal complex (the crystal structure of the complex between NL-2 and NX-1 remains to be determined) was designed, clustered, and used for inducing β-cell functions. Clustering was performed by means of conjugation onto the surface of innovative functional Yb(III) cation-doped maghemite (γ-Fe₂O₃) nanoparticles (NPs) using EDC.HCl activation/coupling (NPs COOH species/functional shell activation), leading to functional nanoscale particulate composites. The resulting peptide-decorated functional NPs exhibited excellent promoting biological activity in β-cells, as described herein. NP-clustered NL-2 mimetics enhance GSIS, protect β-cells under oxidative stress conditions, and increase their proliferation rate. The stimulatory effect of NP-clustered NL-2 mimetics on GSIS was also validated in mouse islets. Based on the observed biological activity of such functional nanocomposites, a novel type of antidiabetic therapy (β-cell protective agent and/or β-cell proliferation inducer) may be developed. In addition, these NL-2 mimetics can be used for the final differentiation step in stem cell-based β-cell production in vitro prior to transplantation. Interestingly, Gjorlund et al. reported in vitro beneficial effects (inducing neurite outgrowth) of another NL-1-derived peptide, CNSP-1 (SEGNRWSNSTKGLFQRA(SEQ ID NO:2)) in neurons. The peptide was also active in clusters (a tetramer coupled to a lysine backbone). Subsequently, this peptide and its modified/shortened version CNSP-2 (HSEGLFQRA (SEQ ID NO:3)) exhibited in vivo biological activity related to the regulation of the social behavior of mice. Both peptides with Ytterbium (III) cation-doped maghemite NPs, but such NP failed to induce any biological effect on the β-cells.

The materials and methods employed in these experiments are now described.

Protein Preparation for Molecular Modeling

Prior to molecular dynamics (MD) simulations, the peptide was capped by an amide group in its C-terminus in accordance with the anticipated peptide synthesis procedure and both the protein (NX-1) and the peptide (HSA-28) were prepared using the Prepare Protein protocol in Discovery studio version 2.5. This protocol inserts missing atoms in incomplete residues, models missing loop regions, and sets the protonation state of titratable residues based on predicted pKa values.

MD Simulations

MD simulations were performed using the Gromacs Molecular Dynamics package (version 4.5) with the AMBER99SB-ILDN force field. The system (a protein and peptide) was submerged in TIP4P water in a dodecahedral box with an extra extension along each axis of a peptide of 10 Å. Ions were added to the solution to make the system electrically neutral. The structure was minimized, equilibrated (first under NVT conditions for 100 ps and then under NPT conditions for an additional 100 ps) and finally simulated under NPT conditions for 100 ns. The simulations were performed at 300° K with a time step of 2 fs using the leap-frog algorithm. The cutoff for van der Waals and Coulomb interactions was set to 10 Å. Long-range electrostatic interactions were computed using Particle Mesh Ewald Summation. Periodic boundary conditions were applied. The LINCS algorithm was used to constrain the bond lengths. The production phase was performed twice, each time starting from a different random number.

Peptide Synthesis

Three peptides were synthesized (QQGEFLNYD (SEQ ID NO:1), SEGNRWSNSTKGLFQRA (SEQ ID NO:2), and HSEGLFQRA (SEQ ID NO:3)) using a solid phase method. Briefly, the synthesis was performed using 0.05 mmol HMBA-AM resin (0.86 mmol/g loading). The resin was loaded with the first AA (10 eq) using DIC (78 mg/0.25 mmol)/DMAP (1 mg-catalytic amount) chemistry. Coupling completion was checked by UV absorption at 299 nm. Fmoc protecting groups were removed by treatment with a 20% piperidine/DMF solution. The remaining AA (4.5 eq) was coupled using BOP (88.4 mmg/0.2 mmol), HOBT (27 mg/0.2 mg), and DIEA (0.052 ml/0.3 mmol). Ninhydrine tests were performed after each coupling. Deprotection of the Trt and OtBu protecting group was carried out using a solution of 95% TFA, 2.5% t-isopropanol-silane, and 2.5% H₂O. Finally, the crude peptide was cleaved by 1M NaOH/Dioxan and neutralized with HCl.

Synthesis of HSA-112

First, 0.4 g (0.2 mmol) of PEG₂₀₀₀ was dried by three cycles of toluene washes with subsequent evaporation. The dry PEG₂₀₀₀ was reacted with 0.04 g of succinic anhydride (0.4 mmol) in 10 ml of dry tetrahydrofuran (THF) and 0.3 ml of dry pyridine under a nitrogen atmosphere at 60° C. The reaction mixture was left stirring for 24 h. After the removal of THF under reduced pressure, the product PEG₂₀₀₀- succinic acid was repetitively precipitated and washed with a large volume of diethyl ether. Next, the purified PEG₂₀₀₀-succinic acid was dissolved in 20 ml of dry THF, followed by the addition of 0.04 g (0.4 mmol) of NHS (0.17 gr), and then 0.08 g of DCC (0.4 mmol). The resulting mixture was stirred for 8 h at room temperature before the removal of insoluble materials. Then the NETS-ester of PEG₂₀₀₀-succinic acid was repetitively precipitated and washed with a large volume of diethyl ether, and the product was dissolved in dioxane. This solution was added to a solution of HSA-28 that was dissolved in NaHCO₃15% in dioxane in a 1:1 ratio. The mixture was stirred at room temperature for 48 hours. The solution was filtered and the solvent water and dioxane were removed. The remaining white powder was dissolved in 30 ml of hot EtOH/CH₂Cl₂, and was filtered again and evaporated. The solid residue was dissolved in hot EtOH/ether solution (5 ml), and left overnight at 4° C. The white precipitate was filtered and further purified by HPLC, using a stepwise gradient of water and acetonitrile, and white crystals were obtained.

Preparation and Characterization of NPs

Yb(III)-Doped Maghemite (γ-Fe₂O₃) NPs

A 20 mL aliquot was taken from the neutral MASSART magnetite NP aqueous suspension prepared according to common Massart et al.'s methodology and ytterbium perchlorate (Yb(ClO₄)₃, 50% wt in ddH₂O, 0.475 mL, 0.742 mmol) was added rapidly to the NPs under an inert N2 atmosphere followed by additional degassed ddH₂O (4.0 mL). Subsequently, analytical grade MeCOMe (4.0 mL) was added, and the mixture was ultra-sonicated (1 h, 0° C.) under an inert argone atmosphere using a high-power sonicator (Sonics®, Vibra cell, 750 Watt, power modulator set at 25%) equipped with a Ti horn. The resulting chemically modified Yb(III)-doped maghemite (γ-Fe₂O₃) NPs were washed with water (3×10 mL) using an Amicon® Ultra-15 centrifugal filter device (100K), at 4,000 rpm, 5-6 min, and re-dispersed in ddH₂O (10 mL). Subsequently, cleaned NPs were centrifuged in a regular tube (10 min, 8,000 rpm) to eliminate micrometer-sized aggregates, whereas the corresponding supernatant phase containing the dispersed cleaned non-aggregated NPs was retained.

Both NP organic shell (polyCOOH shell) derivatizations (coordination/contacting mode—the absence of activating EDC.HCl, & organic shell activation using EDC.HCl), together with UV spectroscopy Kaiser testing for polyCOOH/functionality quantification, were performed as described (Lee et al., 1975, Arch biochem Biophys 171:407-17).

Peptide Conjugation Using EDC.HDl Activation/Coupling Chemistry

First, 6.58 nm-sized Yb(III) cation/complex-doped maghemite NPs (2 mL NPs ddH₂O suspension, Fe=1.543 mg/mL) were placed in a scintillation vial and further diluted to 17 mL using milliQ-purified H₂O. Then, 262 μL (1 eq. relative to the carboxylic acids on the NP surface, as measured by the Kaiser test) of an EDC (1-ethyl-3-β-dimethylaminopropyl) carbodiimide.HCl, 0.001 mmol) solution in milliQ-purified H₂O (0.725 mg/mL) was added to the NPs, and the mixture was shaken for 60 min at 15° C. in an incubator shaker. Then, the peptide (HSA-28, 1.1 mg, 0.988 μmol, 1 eq. relative to Kaiser test-measured carboxylic acids on the NPs surface, dissolved in 4 mL of milliQ-purified H₂O), was added, and the mixture was shaken overnight at 250 rpm at 15° C.

f-NP purification was achieved by three cycles of sequential centrifugal separation-decantation (3,000 rpm, 10 min, 10° C.). The resulting cleaned peptide-conjugated f-NPs were dispersed in 20 mL of H₂O for storage.

The same procedure was performed when using 0.5 eq of EDC (131 μL of 0.725 mg/mL solution, 0.0005 mmol) and the corresponding equivalent quantity of HSA-28 peptide (0.55 mg, 0.0.494 μumol, 0.5 equiv.).

Conjugation of the Active Peptide to PAMAM Nanoparticles

Conjugation of the peptide to PAMAM dendrimer was conducted using maleimide moiety and iminothiolane reagent. The solution containing 9.1 mg (90.9 μmol) of succinic anhydride in 6 ml of anhydrous MeOH was added drop-wise into a solution of 0.012 g (Mw 516, 23.2 μmol) of PAMAM and 40 mg of (0.370 mmol) triethylamine in anhydrous MeOH with stirring. After overnight reaction at room temperature, the solvent was evaporated. 12.8 mg of the above product was than reacted with 2.0 mg (10.4 μmol) of ethyl(dimethylaminopropyl) carbodiimide (EDC) in 4 ml of water for 3 hours. Then 103 mg (92 μmol) of HSA-28 in 1 ml DMSO was added drop-wise to the above solution and stirred overnight. The crude product was dialyzed (MWCO=1500 Da).

Determination of the Amount of Conjugated HSA-28 to NPs by Analytical HPLC

In order to investigate the amount of HSA-28 that was conjugated with nanoparticles, the starting solution of the reactions and all washes were analyzed by analytical HPLC: the concentration of HSA-28 was determined. The calibration curve was used for the determination. The curve was built by measuring the known HSA-28 concentrations (1, 0.1, 0.01, 0.001, and 0.0001 mg/ml). HPLC analysis was performed using a C18 reverse phase column (Phenomenex Luna 5u C18(2); 100 A; 250×4.6 mm) with a Young Lin instrument; YL 9100 HPLC series system attached to a chromatograph manager. A gradient was applied between solvent A (H₂O) and solvent B (CH₃CN). The gradient was A/B 0 min [100/0], 20 min [0/100], 20-25 min [100/0]. The flow rate was 1 ml/min. λ=220.

MTT Cell Viability Test

Briefly, cells were incubated with MTT (2 mg/mL) in growth medium for 30 min at 37° C. The medium was then aspirated, and DMSO was added to solubilize the cells and colored crystals. Absorbance at 570 nm was measured in a SpectraMax M5 spectrophotometer (Sunnyvale, Calif., USA). The obtained results were normalized by total protein content in culture cells, which was measured using the Bradford reagent.

Cell Counting

Cells were detached by trypsin and colored by Trypan blue. Only uncolored cells were counted. Cell counting proceeded according to Abcam (Cambridge, Mass., USA) online protocol for work with a haemocytometer.

Cell Lines

INS-1E cells were grown in DMEM (22.5 mM glucose) supplemented with 10% fetal calf serum (FCS), 1 mM glutamine, and antibiotics (100 m/mL penicillin, 100 m/mL streptomycin) at 37° C. in a 5% CO2 humidified atmosphere, as described by elsewhere (Pasternack et al., 2014, Chem Commun 50(76):11222-5). PC-3 and PC-12 were grown and maintained as previously described.

Cell Lysate Preparation

The lysis buffer contained 50 mmol/L Tris-HCl, pH 7.5, 1 mmol/L EDTA, 1 mmol/L EGTA, 1 mmol/L Na3VO4, 150 mmol/L NaCl, 50 mmol/L NaF, 10 mmol/L sodium-glycerophosphate, 5 mmol/L sodium pyrophosphate, and 1 mmol/L PMSF, supplemented with 0.1% (v/v) IGEPAL, 0.1% (v/v) 2-β-mercaptoethanol, and protease inhibitor cocktail (1:100 dilution). The cells were washed with ice-cold PBS, and 1 ml of lysis buffer was then added at 4° C. for 40 min. The resulting cell lysates were centrifuged at 7,800×g for 30 min at 4° C., and the supernatant fractions were separated and kept at −70° C. until used. Protein content in the supernatant was determined by Bradford analysis, using a BSA standard dissolved in the same buffer.

GSIS and Insulin RIA

The assays were performed for INS-1E medium and lysates as previously described (Pasternack et al., 2014, Chem Comm 50:11222-5).

Induction of Oxidative Stress

Oxidative stress conditions were induced by supplying glucose oxidase (GO, 50 mU/ml) with high levels of glucose (23.5 mM) to the growing medium of the INS-1E cells. This resulted in an elevated H₂O₂ concentration in the medium (reaching 29.0±9.6 μM in 4 h of incubation). The concentration of H₂O₂ generated by the glucose oxidase/glucose system was determined as described by Thurman et al. (1972, Eur J Biochem 25:420-30).

Fluorescent Microscopy

INS-1E cells were grown in 6-well plates. HSA-28P labeled by FITC was added to cells for 24 h (control cells were treated only with FITC-labeled nanoparticles). After incubation, cells were gently washed two times with pre-warmed growth medium to remove unbound labeled nanoparticles. After the cells were washed, they were visualized with a CellsSense Live Imaging microscope (Olympus, Tokyo, Japan) operated at 37° C.

Confocal Microscopy

Cellular Localization of HSA-28P

Experiments were conducted in INS-1E cells that were seeded on coverslips in 6-well plates. The cells were incubated with HSA-28P labeled by FITC for 24 h (control cells were treated with naked nanoparticles labeled by FITC). Following incubation, the slides were washed three times with pre-warmed PBS and fixed with formaldehyde (4% in PBS). Subsequently, slides were washed three more times with pre-warmed PBS. The membranes of cells were stained with Alexa Fluor 633 Phalloidin according to the protocol provided by the manufacturer. Slides were washed three times by pre-warmed PBS. Nuclei were stained with DAPI according to the protocol supplied by the manufacturer. Fluorescent signals were visualized with a Confocal-Zeiss microscope equipped with a 60×/1.4 objective (Oberkochen, Germany).

The Effect of HSA-28P on the C-Peptide Level

Cells were grown as described above. Slides were incubated with HSA-28P or with non-coated nanoparticles for 24 h. The level of C-peptide was determined by immunocytochemistry using anti-C-peptide antibody. Thereafter, slides were fixed with formaldehyde as described above. Cell membranes and nuclei were stained as previously described. Visualization of fluorescent signals proceeded as described above.

The Effect of HSA-28P on the Glucagon Level

Cells were grown as described above. Slides were incubated with HSA-28P or with non-coated nanoparticles for 24 h. The level of glucagon was determined by immunocytochemistry using anti-glucagon antibody. Thereafter, slides were fixed with formaldehyde as described above. Cell membranes and nuclei were stained as previously described. Visualization of fluorescent signals proceeded as described above. Glucagon that was conjugated to the polylysine layer was used as a positive control. Slides were covered by polylysine according to the instructions of the product's manufacturer.

Effect of PAMAM based nanoparticles covered by HSA-28 on PDX1 Pdx1 (Pancreatic and duodenal homeobox 1) is a transcription factor which necessary for pancreatic development and β-cell maturation. Diabetes type 2 is characterized by Pdx1-deficient β cells. INS-1E cells were incubated for 72 h with HSA-28D ([HSA]-0.003 mg/ml). Cells were fixed with formaldehyde (4% in PBS), permeabilized with 0.2% Triton X-100 and exposed to anti Pdx1 antibody (Abcam-ab47267) followed by secondary antibody (Abcam-ab150081) according to manufacturer's protocol. The membranes of cells were exposed to Alexa Fluor 633 Phalloidin (Rhenium-A22284) and nuclei were stained with DAPI (Sigma Aldrich-F6057). Fluorescent signals were visualized with a Confocal-Zeiss microscope; the identical optical and imaging conditions were kept during experiments and visualization work. The experiment was run several times using triplicates, n=6.

Mice

C57 black mice were purchased from Jackson Laboratories (Bar Harbor, Me., USA). The animals were housed under standard conditions: constant temperature (22±1° C.), humidity (relative, 40%), and a 12 h light/dark cycle and were allowed free access to food and water. Bar-Ilan University is an Association for Assessment and Accreditation of Laboratory Animal Care (AAALAC) internationally accredited institution. “Bienta” works under Ukrainian veterinary regulations.

Isolation of Mouse Islets

Mice (6 in total) were euthanized by cervical dislocation; the mouse torso was saturated with 70% ethanol. The abdominal cavity was opened completely from anus to diaphragm. The body and tail of the pancreas were removed and separated from fat and large vessels. The pancreas was washed once with HBSS solution and cut into small pieces of approximately 2×3 mm with a scissors. After an additional wash with HBSS solution, the pieces of pancreas were placed in two 50 ml Falcon tubes containing 4 ml of a freshly prepared solution of collagenase (2 mg/ml). After vigorous shaking at 37° C. for approximately 20 min, the collagenase digestion was interrupted by adding 45 ml of ice-chilled HBSS solution. The digested pancreatic tissue was spinned down at 330×g for 3 minutes. Supernatant was removed again and an additional 45 mL of “islet media” was added and spinned down at 330×g for 3 minutes. The histopaque was decanted and the tubes were inverted to drain on an absorbent paper towel for 1 min. The pellet was resuspended in 20 ml for each tube in cold 30% Ficoll solution. The histopaque was overlaid with 10 ml of HBSS and centrifuged at 900×g for 12 min. The islets floated at the interface of the Ficoll and media. Islets were collected from the interface, and transferred to a fresh 50 ml tube. Islets were washed twice with HBSS solution and spinned down at 330×g for 3 minutes. The resulting pellet was suspended in 10 ml “islet medium” and passed through an inverted 100 μm filter and then through an inverted 30 μm filter. Islets were rinsed into a Petri dish by pipetting 10 ml of the RPMI 1640 medium supplied with 10% heat-inactivated FBS, 1% Pen-Strep solution, and 11 mM glucose through the filter while holding it right side up over the dish. Around 300 islets per dish were seeded. Islets were then cultured at 37° C. with 5% CO₂ for 2 days.

Glucose-Stimulated Insulin Secretion (GSIS) Test in Islets

After incubation with test compounds in RPMI 1640 medium, the islets were taken to GSIS. The medium was changed to KRBB supplemented with 2.5 mM glucose (minimal volume). Islets were preincubated in KRBB for an additional hour and then the medium was changed to either 2.5 mM or 16.7 mM glucose KRBB medium. Islets were incubated for 1 hour in different glucose concentrations. 10 mM of L-Arginine was used as a positive control. At the end of incubation, the medium was collected and spinned down at 500×g for 5 minutes. The supernatant was used for determining the insulin level in the medium. Islets were washed by cold KRBB and lysated by adding lysis buffer (RIPA buffer supplemented with protease inhibitors) and shaken for 30 minutes in a cold room. Using “policemen”, the lysate was collected and spinned at 10.000×g for 10 minutes. The resulting supernatant was used to determine the insulin contents and the total protein level.

Determination of the Insulin Level in Mouse Islets

Frozen islets medium or lysates were thawed on ice and diluted with water (a typical dilution for the lysate was 300× and for the supernatant—5×). Mouse Ultrasensitive Insulin ELISA kit, ALPCO (Salem, N.H., USA) was used for insulin level determination as described in the manufacturer's protocol. The signal was determined by a SpectraMax M5 spectrophotometer. The concentrations of insulin in the samples were normalized by the total protein determined by the BCA assay.

Low Doses Streptozotocine (LDSTZ) Treated C57 Black Mice.

Streptozotocine (50 mg/kg) was injected IP in citrate buffer (pH 4.5) IP to after overnight fast. Hyperglycemic mice were taken to the experiment.

IP Injection of Both Types of Nanoparticles to LDSTZ Treated Mice.

During 6 days, HSA-28D (1.51 mg/kg), HSA-28, “naked” nanoparticles and PBS were injected IP and morning blood glucose level was tested by glucometer (tail bleeding test).

Statistical Analysis

Statistical significance *, # (p<0.05) was calculated among the various experimental groups using Student's t-test (two tailed). Results are given as the mean±SEM (n=3-6). The QuickCalcs online service (GraphPad Software: http://www.graphpad.com/quickcalcs/ttest1.cfm) was used for statistical evaluations.

The results of the experiments are now described.

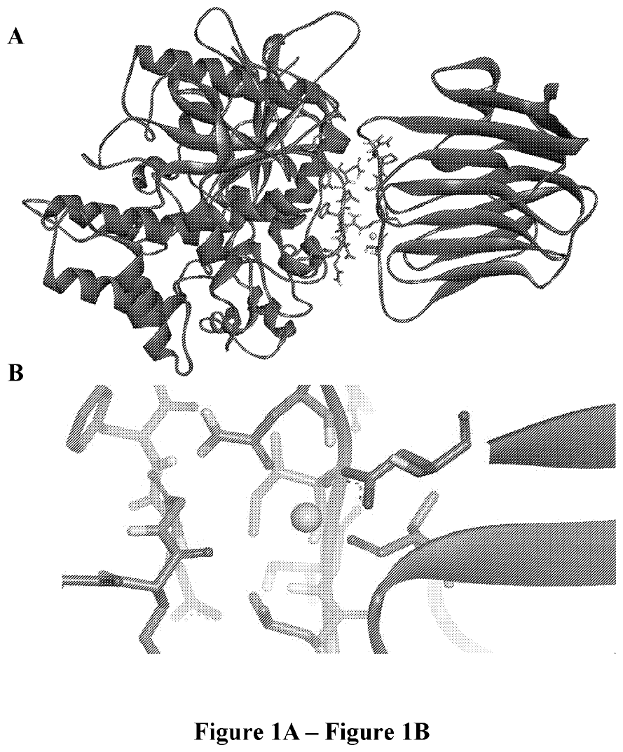

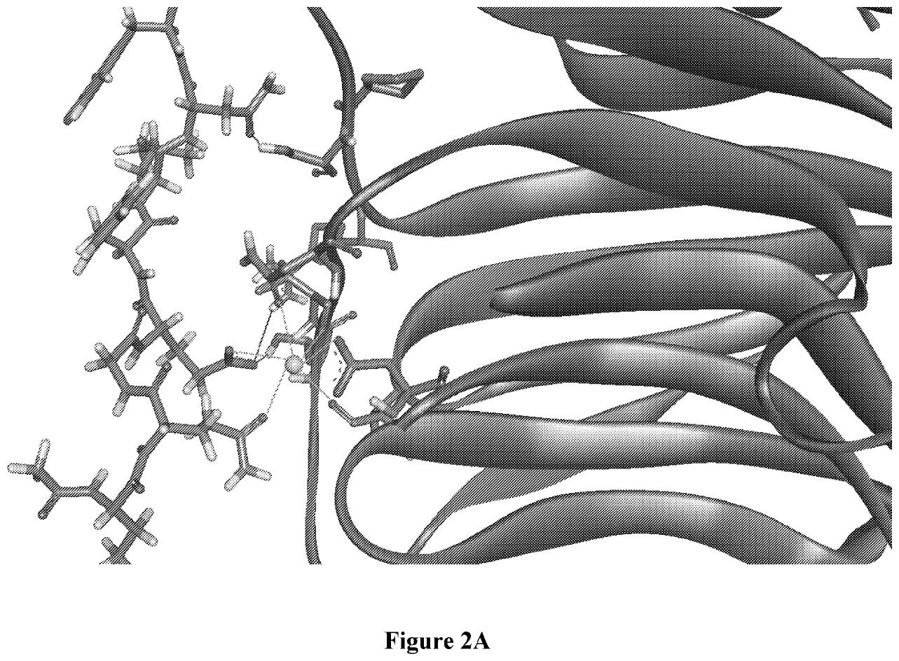

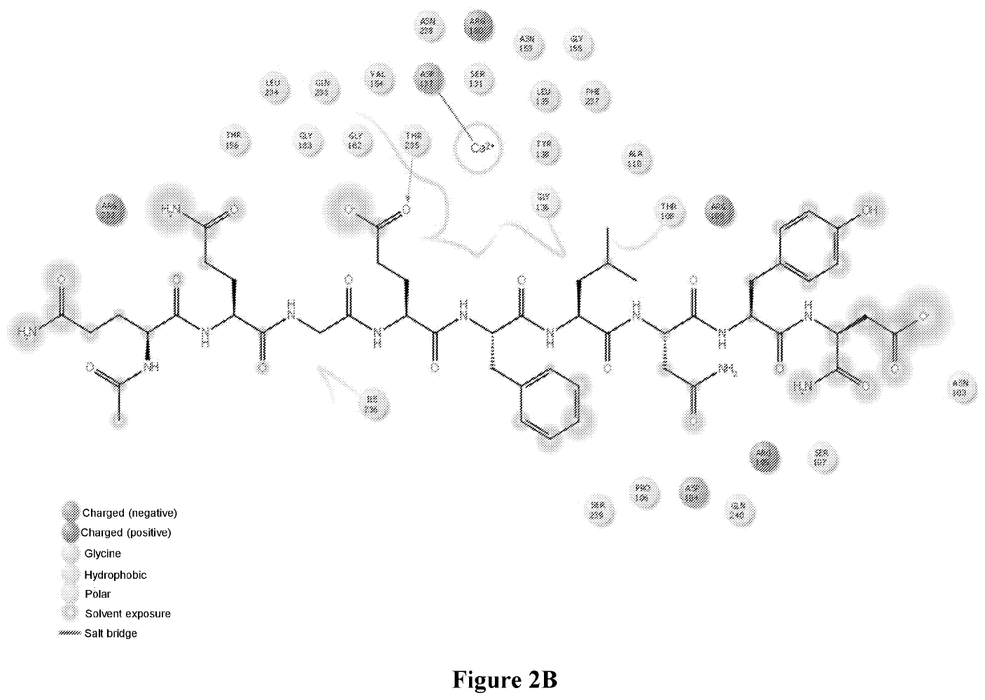

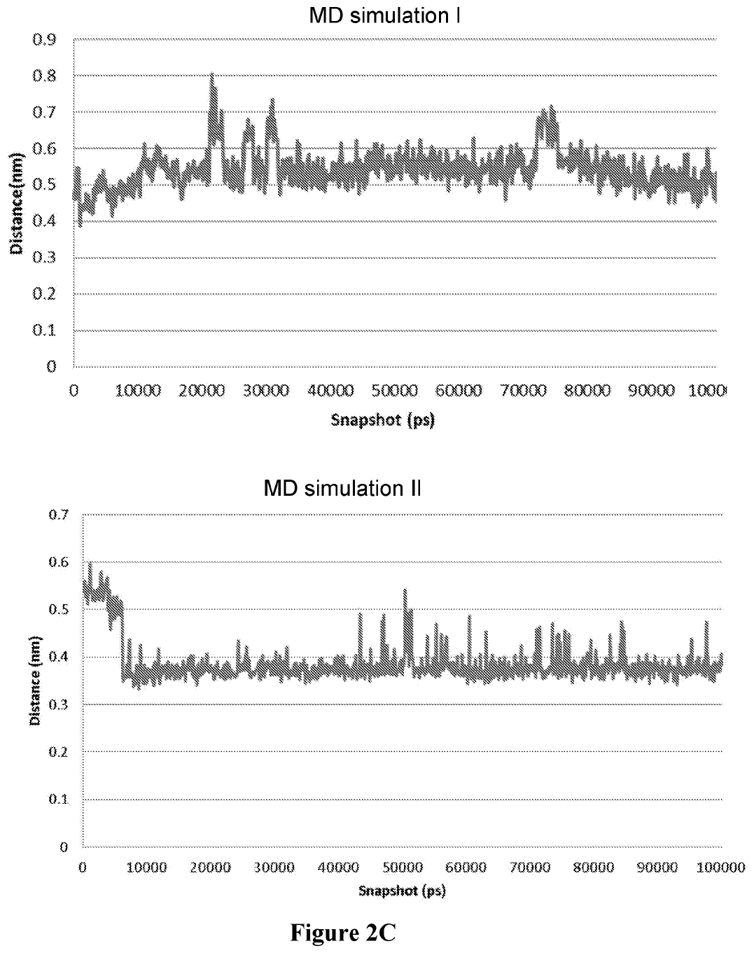

Owing to the lack of a crystal structure in the NL-2/NXs complex, the homologous NL-4/NX1 complex (PDB code 2WQZ; the sequence identity between hNL-2 and hNL-4 is 65.2%) was used as a starting point for designing the peptide. The interactions between NX-1 and NL-4 are mediated directly by four hydrogen bonds, namely, NX-1 Thr 235 with NL-4 Glu361, NX-1 Pro 106 with NL-4 Asn 364, NX-1 Ser 107 with NL-4 Asn 364, NX-1 Arg 109 with NL-4 Asn 364, and by favorable VdW interactions between NX-1 Pro 106, Thr 108, Leu 234, Ile 236 and NL-4 Gly 360, Phe362, Leu 363, respectively, as shown in FIG. 1A. In addition, a calcium ion indirectly coordinates with residues Asp 137, Asn 238, Val 154, and Ile 236 of NX-1 and residues Gln 359 and Gly 360 of NL-4 (FIG. 1B). The only difference between the sequences of NL-4 and NL-2 at the interface with NX-1 is at position 358 (glutamate in NL-4 vs. glutamine in NL-2) and this mutation was manually made. Upon visual inspection of the NL-4/NX-1 interface, a nine-residue peptide (Q358-Q359-G360-E361-F362-L363-N364-Y365-D366, and HSA-28) was selected from the sequence of NL-4 as a potential binding partner of NX-1. The HSA-28 peptide was evaluated for its ability to stably interact with the NX-1 interface through MD simulations. Two independent 100 ns MD simulations were performed for the NX-1/HSA-28 system, each initiated with a different random number. Throughout both simulations, the peptide remained in proximity to the protein primarily by maintaining the coordination with the calcium ion (FIG. 2A and FIG. 2B). Based on the chelating distance between HSA-28 and the calcium ion, it was concluded that their interaction is stable (FIG. 2C). These findings therefore suggest that the designed peptide can indeed bind NX-1.

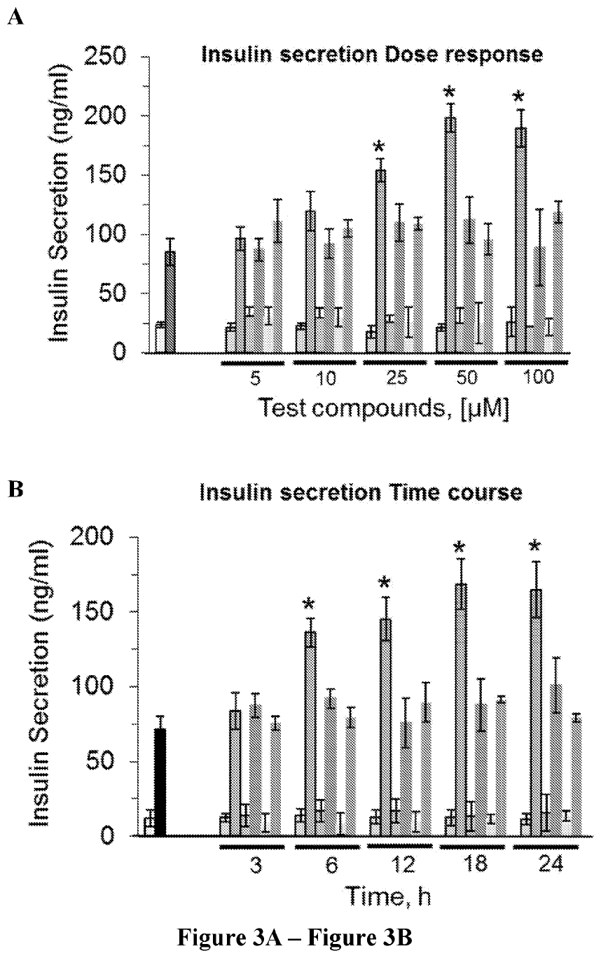

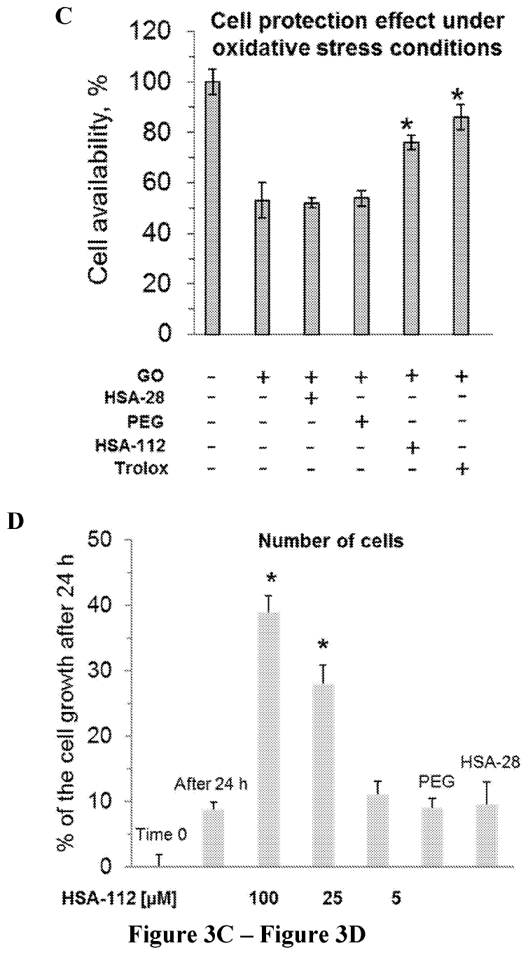

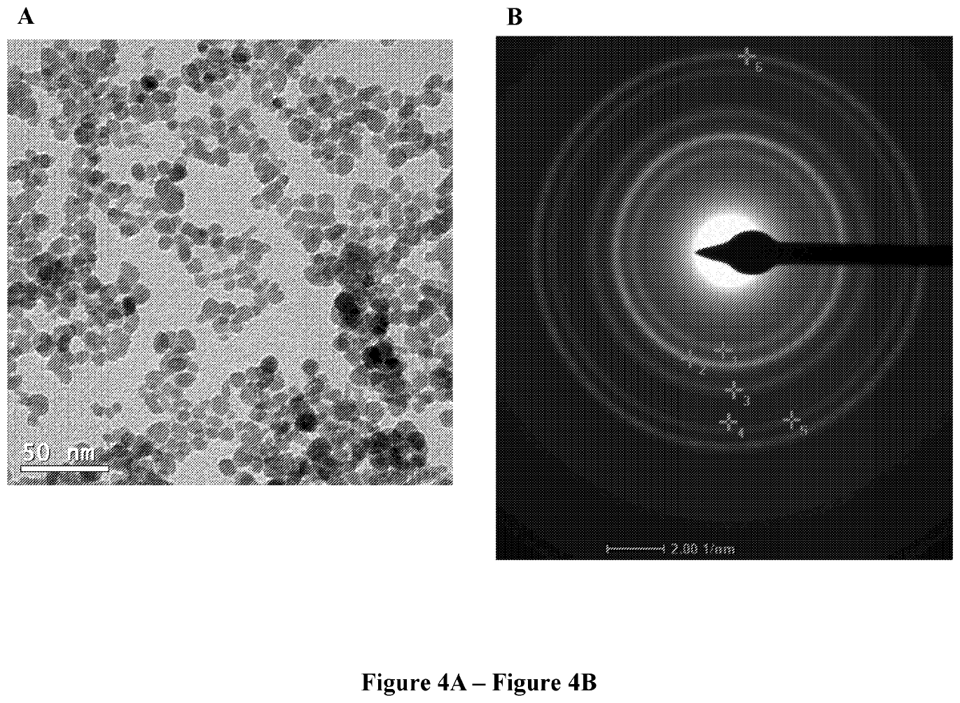

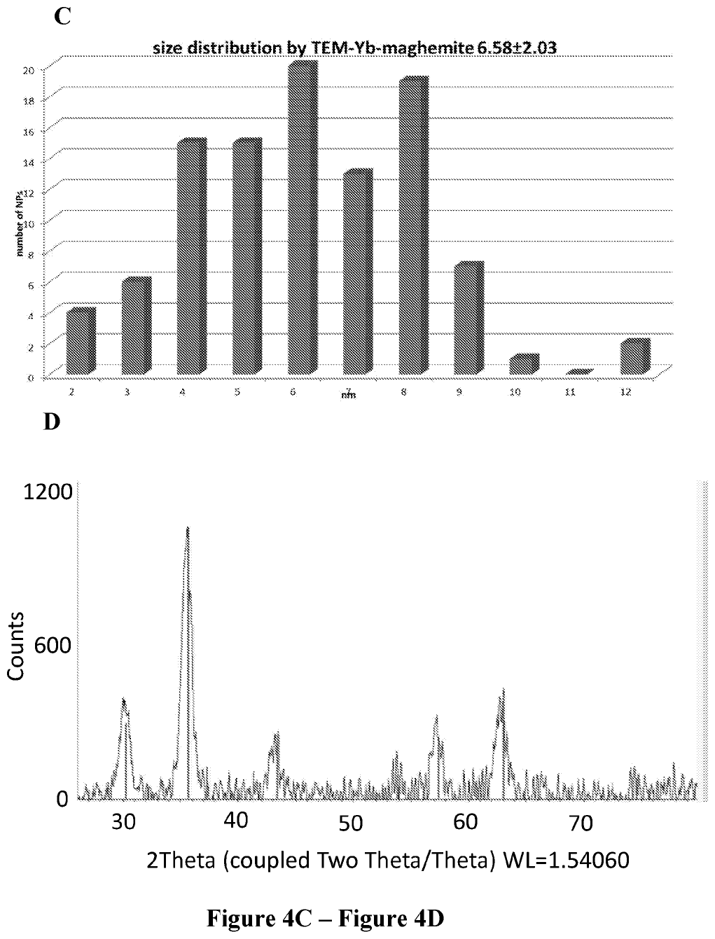

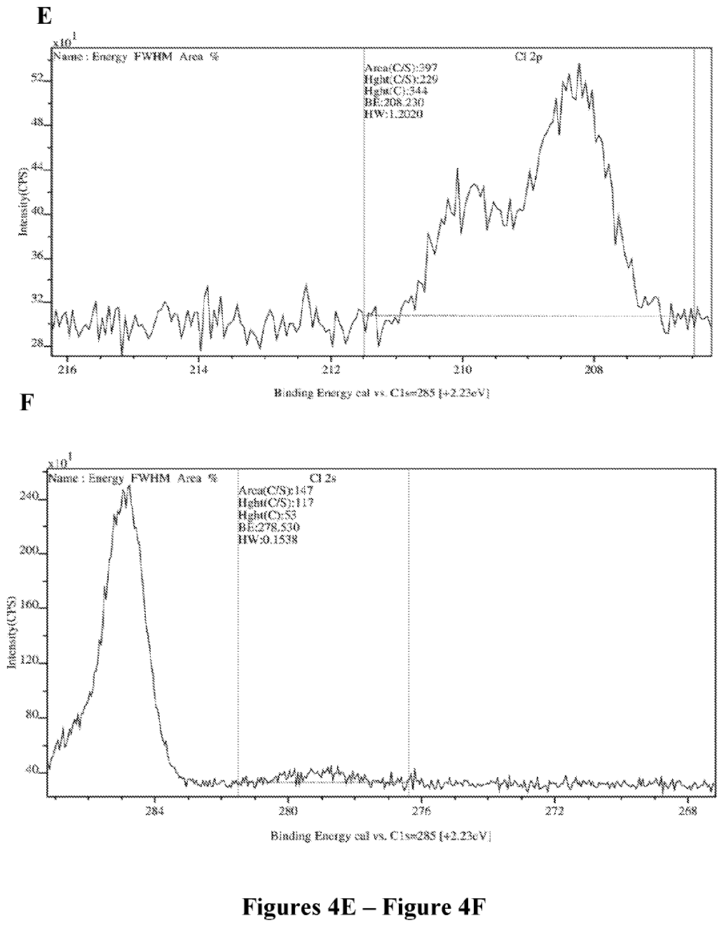

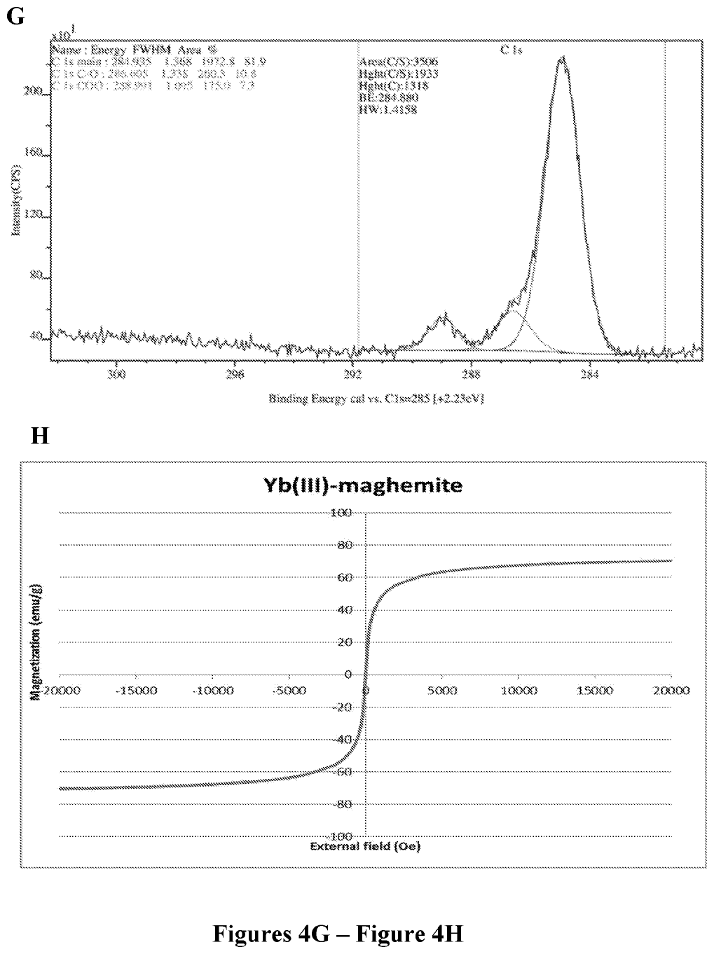

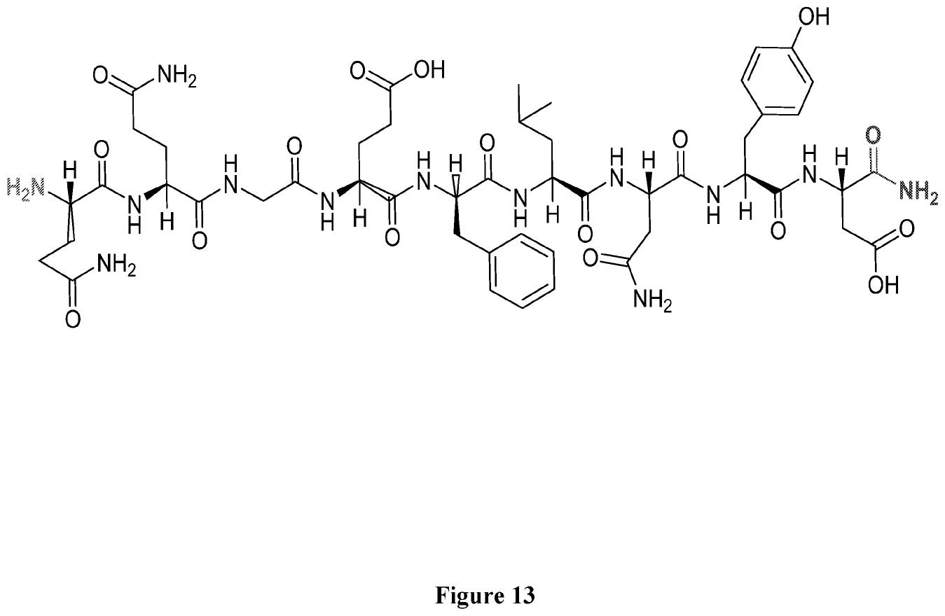

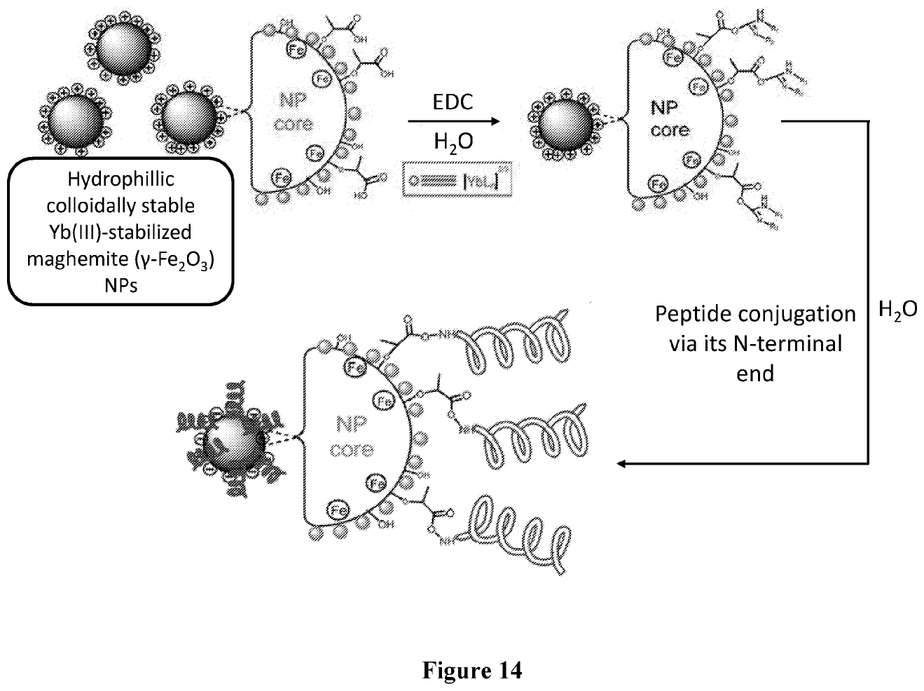

The HSA-28 peptide was synthesized with an amide moiety in its C-terminal (FIG. 13) and tested in INS-1E β-cells. The isolated peptide did not increase the GSIS and has not shown any effect on the proliferation rate of the cells (data not shown). However, an HSA-28 dimer formed by bridging two-HSA-28 monomers via amide bond formation with active ester PEG_{2,000} (HSA-112), dose-dependently increased GSIS in INS-1E cells (FIG. 3A), with an EC₅₀ of 32 μM. The effect of HSA-112 was also time dependent: after 6 hours, the compound at a 50 μM concentration induced a significant increase in insulin secretion, which lasted for 24 h. The maximal stimulatory effect of the compound was observed after 18 h and consisted of a twofold elevation of GSIS parameters (FIG. 3B). HSA-112 did not affect the cellular insulin content (data not shown). In addition, HSA-112 exhibited a cytoprotective effect on glucose oxidase-induced oxidative stress. The effect of the compound on cell viability was almost identical to that of the well-known antioxidant (trolox), which was used as a positive control (FIG. 3C). Since HSA-112 did not seem to be an antioxidant, it was hypothesized that cell protection resulted from the ability of the compound to induce β-cell clusters, which are much more resistant to any kind of damage including oxidative stress, than are dispersed cells. Simple cell counting revealed that indeed the amount of cells in the presence of HSA-112 was higher than that in the control dishes (FIG. 3D). Despite the good biological activity of HSA-112, its in vitro active concentration, 50 might be too high for further development of in vivo active molecules. In addition, the lack of an effect on the insulin content was an additional source of concern. Having observed a positive effect of HSA-28 upon dimerization via the PEG linker, a poly-HSA-28 cluster was generated by covalent linking the peptide onto the maghemite-based NP surface via its N-terminus (FIG. 14). Consequently and similarly to the formerly described Ce^{3/4+}-cation-doped γ-Fe₂O₃ NPs, pre-formed Massart magnetite NPs were reacted with Yb(III)-perchlorate [Yb(ClO₄)₃] in an inert argon atmosphere using a high power Ti horn probe sonicator to produce corresponding Yb′ cation-doped maghemite (γ-Fe₂O₃) NPs functionalized by a polycarboxylic acid (polyCOOH) shell. Selected significant characterization data have been gathered, as shown in FIGS. 4-6. A TEM microphotograph (FIG. 4A) was combined with a SAED X-ray (FIG. 4B) diffraction pattern and confirmed the crystallinity of Yb³⁺ cation-doped γ-Fe₂O₃ NPs while providing an average 6.58±2.03 nm-sized NP diameter (NP TEM size histogram, FIG. 4C). An XRD spectrum (FIG. 4D) of the NP surface doping by Yb(III) cations/complexes (perchlorate coordinated ligands, elemental Yb presence) has been confirmed by elemental TEM-EDAXS (Yb(III) L: 1.92 atomic %) and ICP-AES (Jobin Yvon Ultima 2, see the complete quantitative data in Table 1). Moreover, elemental Yb could not be directly detected by surface-sensitive XPS due to its low level of NP doping. However, this same NP surface analysis method enabled easy detection of both (i) Yb(III)-coordinating perchlorate ligands (FIG. 4E and FIG. 4F, XPS, Cl₂ₛ & Cl₂ₚ peaks: binding energies of 278.530 & 208.230 eV, respectively), and of (ii) the organic ultrasound-generated polyCOOH shell (FIG. 4G XPS, C₁ₛ (polyCOOH functional shell): binding energy of 288.991 eV). Quantitative confirmation of the presence of this organic polyCOOH functional shell has been further obtained by using a differential sensitive ninhydrin-based UV spectrophotometric Kaiser test with coupling of 1,4-diaminobutane in excess with and without polyCOOH activation by EDC.HCl carbodiimide. This measurement provided a 0.129 mmol concentration of COOH groups (polyCOOH shell)/g on the surface of the NPs, which is useful for variable underlayer/uplayer 2^{nd} step quantitative ligand attachment onto the NP surface. Yb³⁺ cation-doped γ-Fe₂O₃ NPs have also been characterized by SQUID magnetization profile analysis at 298° K (FIG. 4H), demonstrating no hysteresis, as expected for superparamagnetic NPs and a saturation magnetization value, Ms, of 70.4 emu/g NPs.

TABLE 1

| | Yb(III) cation-doped |
|---|---|
| Nanoparticle | maghemite NPs |
| Mean hydrodynamic diameter by DLS (nm) | 42 |
| Size by TEM (nm) | 6.58 ± 2.03 |
| Zeta potential [mV] | (+)46; (+) 50 |
| Fe [mg/mL] using ICP or AAS | 1.476 |
| Doping metal [mg/mL] using ICP or AAS | 0.272 |
| Doping metal to Fe Weight ratio - wM/Fe | 0.184 |
| Doping metal to Fe Molar ratio - mM/Fe | 0.059 |

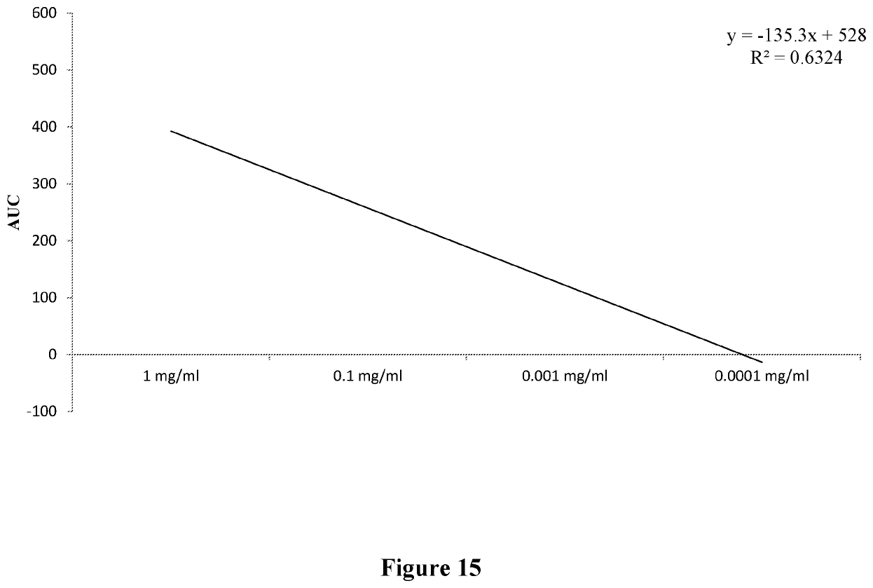

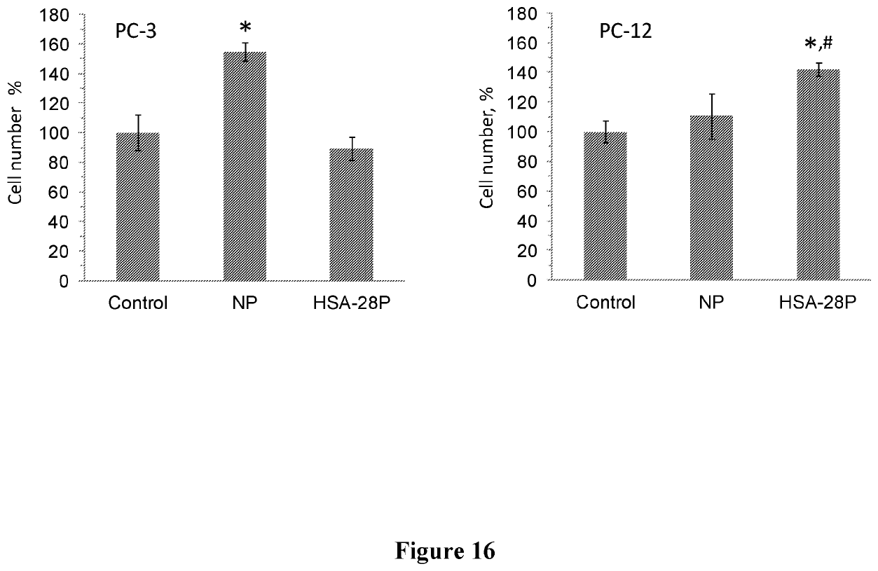

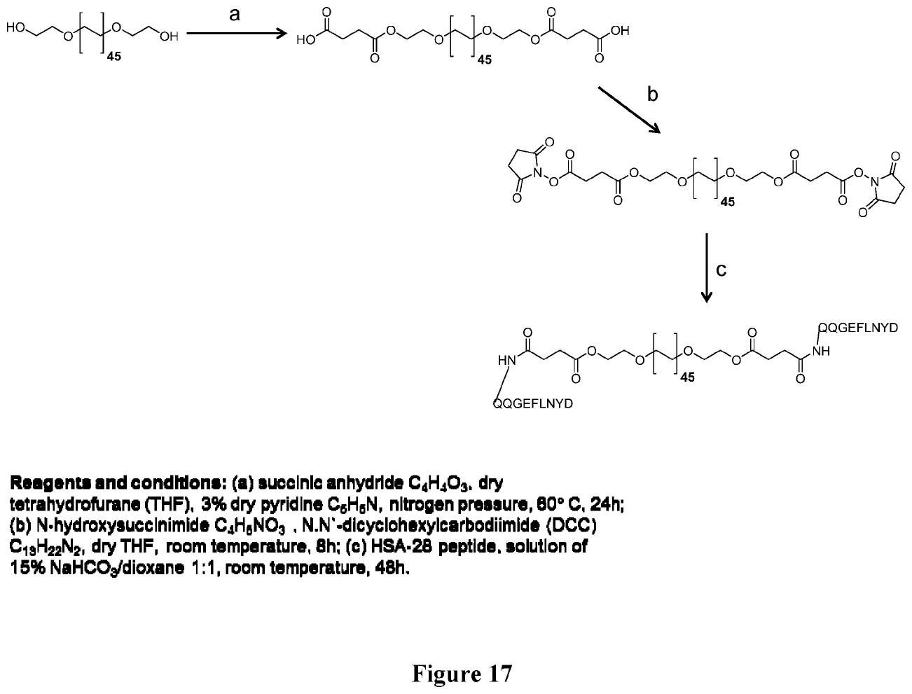

In a 2^{nd} step of NP covalent decoration, the HSA-28 peptide was covalently conjugated onto the surface of functional Yb(III)-doped γ-Fe₂O₃ NPs via N-terminus amide bonding using EDC.HCl carbodiimide polyCOOH shell activation/coupling, leading to functional nanoscale composites of the peptide (FIG. 14) The final concentration of the peptide was determined by HPLC, as shown in FIG. 15. For HSA-28P the concertation of the conjugated peptide was 0.988 mM and for HSA-28P1/2 it was 0.494 mM, respectively

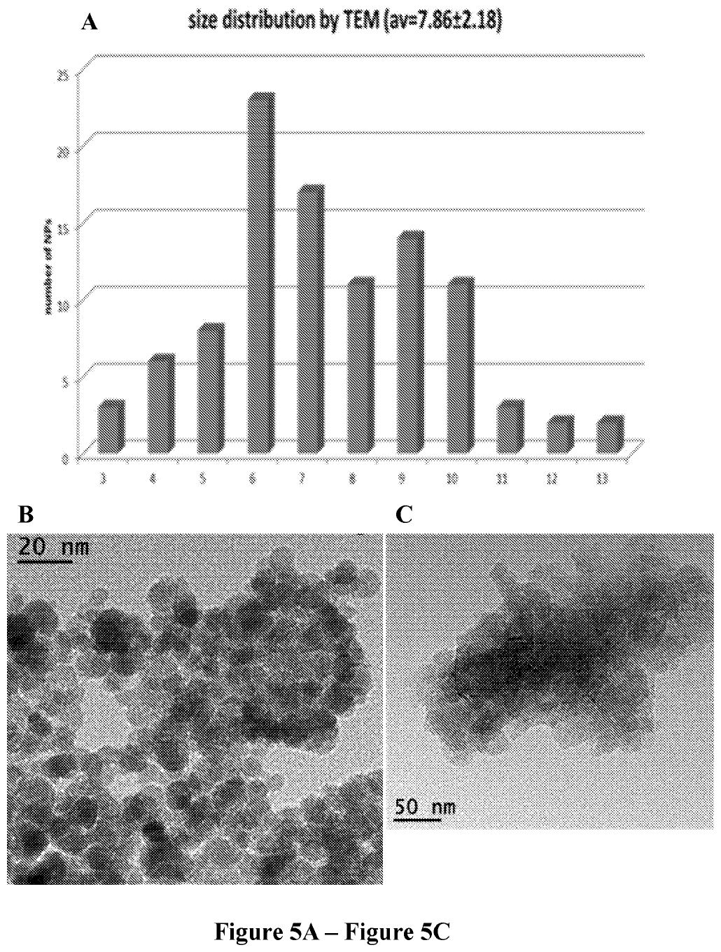

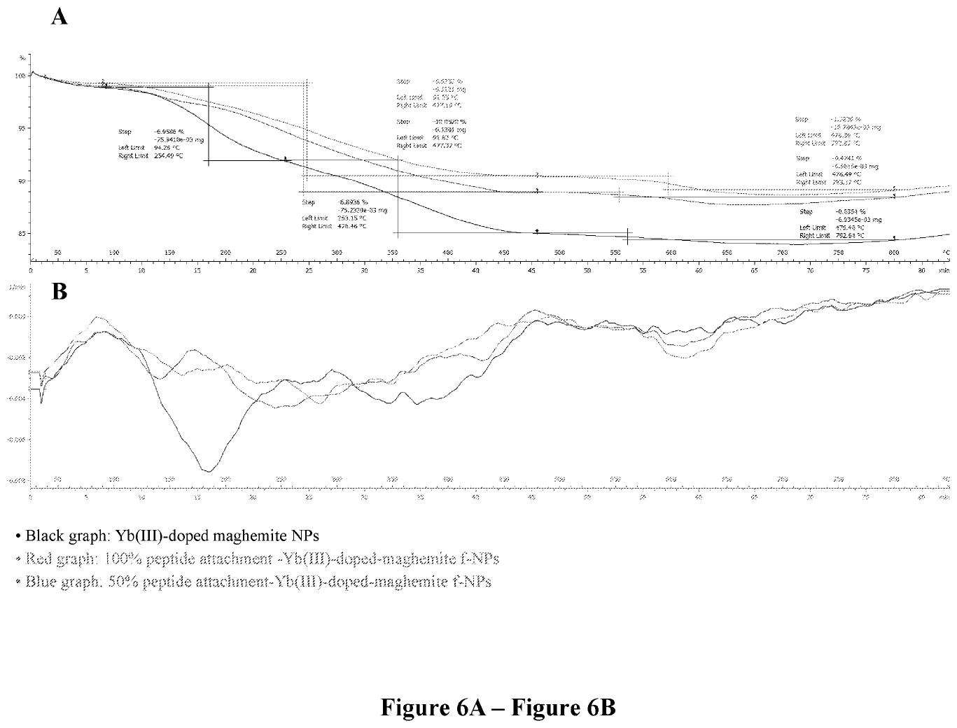

For this purpose, two different quantities of EDC.HCl conjugated peptide were used, based on the former Kaiser polyCOOH shell quantification, i.e., 100% (1.0 eq vs. NPs surface COOH quantity, 1.1 mg HSA-28 peptide, 0.988 μmol) and 50% (0.5 eq vs. NPs surface COOH quantity, 0.55 mg, 0.494 μmol) of the total quantity of carboxylic acids (polyCOOH shell) present on the NP surface. The TEM microphotographs (100% COOH functions for peptide attachment) and the TEM-derived size distribution (7.86±2.18 nm) of the resulting functional peptide-decorated NPs (f-NPs) are reported in FIG. 5. In addition, thermogravimetric TGA analyses were performed (FIG. 6A), and revealed total weight losses of both peptide-conjugated f-NPs in comparison with core Yb(III)-γ-Fe₂O₃NPs (FIG. 6B). Such weight losses (core Yb(III)-γ-Fe₂O₃, 13.85%-T° C. range: 94.28-253.15° C.; 50% f-NPs, 10.05%—T° C. range: 91.62-477.37° C.; 100% f-NPs, 8.8352%—T° C. range: 91.55-477.19° C.) mainly accounted for HSA-28 peptide covalent attachment. Note that the covalent attachment of approximately 50% and 100% peptide to the functionalized NPS most likely led to the removal of weight-increasing polyCOOH organic layer components from the f-NPs surface, thus providing a plausible explanation as to why TGA analyses were unable to quantify the exact quantities of linked peptide onto the surface of respective f-NPs. In order to further track the effective peptide covalent attachment, both DLS and ζ potential measurements were also performed. Whereas core Yb(III)-γ-Fe₂O₃NPs displayed a DLS value of 42.0 nm and a highly positive ζ potential in the +46-+50 mV range, both 100% and 50% peptide-conjugated-f-NPs displayed higher average DLS hydrodynamic diameter values in the micrometer range (2,062 and 2,357 nm, respectively) as well as negative ζ potential values (−15.1 and −16.0 mV, respectively).

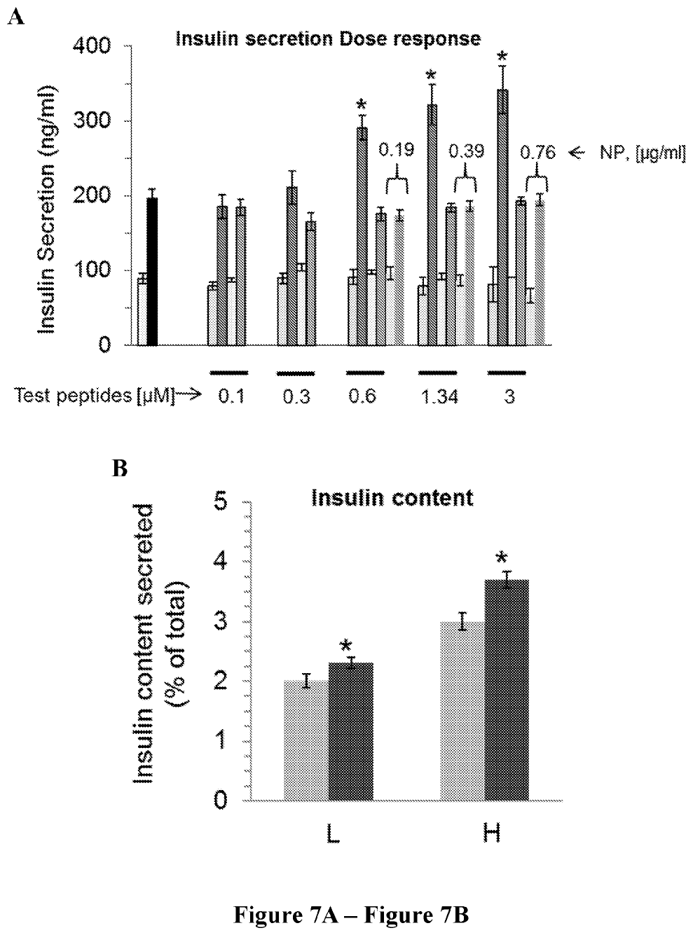

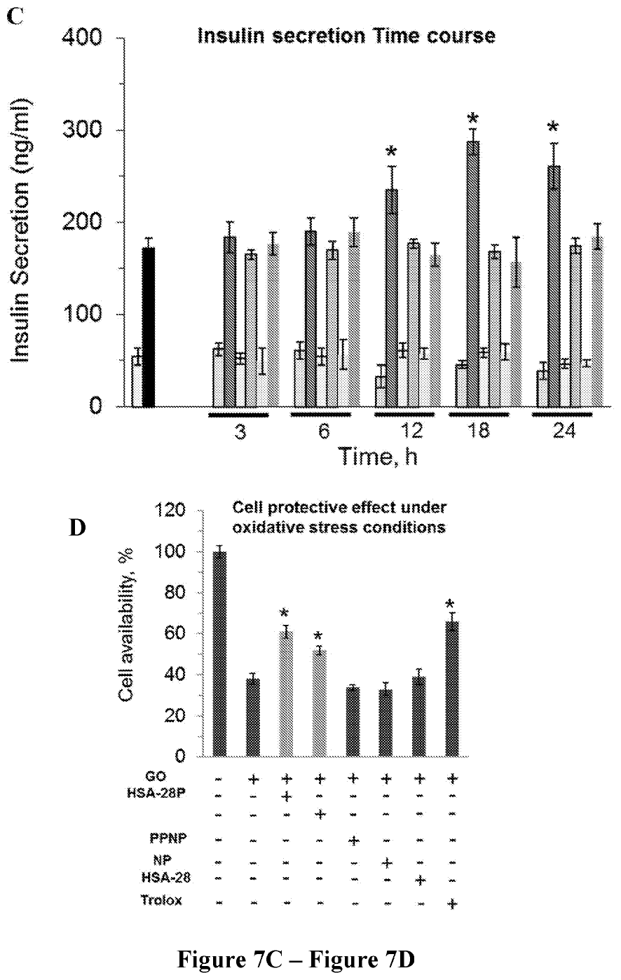

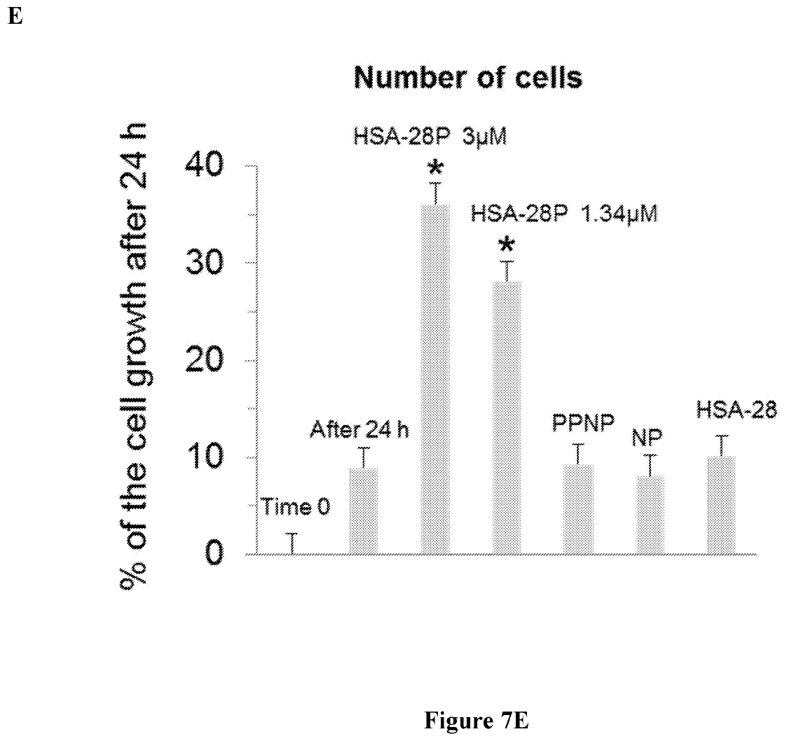

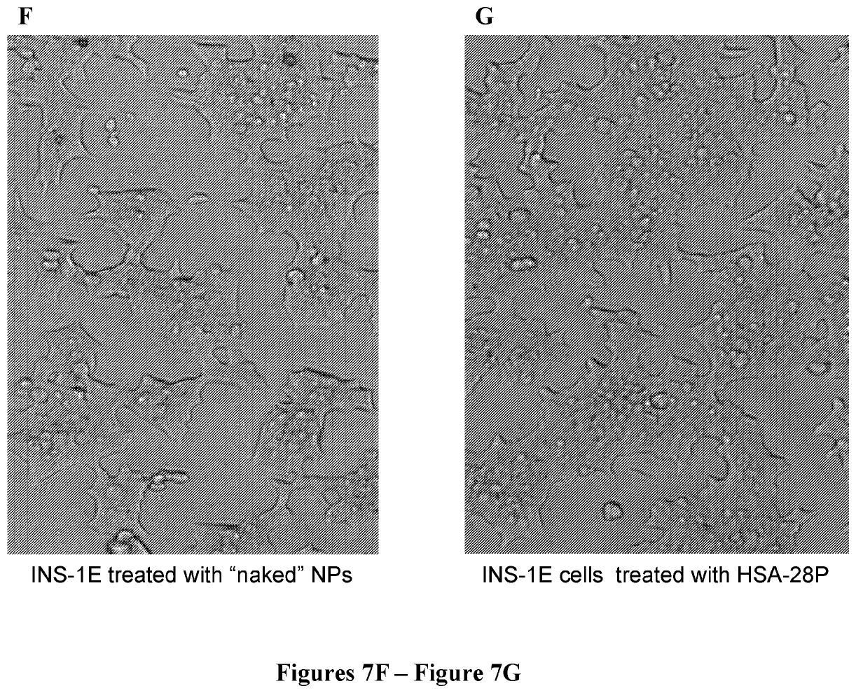

Similar to the original peptide, the biological evaluation of HSA-28P was also performed in a rat INS-1E cell line. HSA-28P dose- and time-dependently enhanced GSIS was compared with untreated cells and control cells treated with the “naked” NPs and by identical NPs covered by a “scrambled” peptide (FIG. 7A and FIG. 7B). The maximal stimulatory effect on GSIS was obtained after 18-24 hours of incubation at a concentration of 3 μM/per μg of NPs/ml. Importantly, the stimulatory effect on GSIS was significant already at a concentration of 0.6 μM/per μg NPs/ml of HSA-28P. The total insulin content was also significantly elevated by HSA-28P in both (low and high) glucose concentrations after 24 h of incubation (FIG. 7C). The effect of HSA-28P on INS-1E β-cells' viability under oxidative stress conditions (glucose oxidase/glucose system) was further investigated. After 24 hours INS-1E cells that were placed in plates together with HSA-28P (3 μM/per μg of NPs/ml) exhibited higher viability (dose response behavior) than all the tested controls did (FIG. 7D). This cell protective effect was equal to the effect that was induced by trolox. Finally, the number of cells was also significantly higher in HSA-28P-treated samples compared with the control (FIG. 7E). Likewise, the effect of the compound on the cell number was dose dependent. In addition to the increased proliferative rate, the compound induced the formation of β-cell clusters, as shown in FIG. 7F and FIG. 7G. The possible proliferative stimulation of non-β-cells by HSA-28P was also investigated. Two types of cells, PC-3 (human prostate cancer cells) and PC-12 cells (rat pheochromocytoma) were chosen for this control experiment. No significant effect of HSA-28P was observed in PC-3 cells (FIG. 15), yet similarly to its effect on β-cells, HSA-28P increased the proliferation of PC-12 cells (approximately by 40%). Such results are not surprising due to the presence of neuroligines and neuroxines in the plasma membrane of neuronic PC-12 cells. These data indicate that the ability of HSA-28P to stimulate cell growth is specific to cells that were developed from the ectodermal/endodermal precursor population common to neurons and β-cells.

Consistent with the explanation, NPs decorated with HSA-28 should interact with NXs that are located on the plasma membrane of the INS-1E cells and should cause them to adhere to each other to form functional cell clusters. FITC-labeled HSA-28 NPs were fabricated. A commercially available lysine (protected in its amine terminal by a 4-methyltrityl group) was conjugated with the last amino acid in the sequences of HSA-28. After the removal of the protective group, the free primary amine was reacted with FITC-isothiocyanate isomer I. Next, the labeled peptide was cleaved from the resin and used for the coupling with core Yb(III)-doped maghemite NPs, as described above. However, this composite led to a very weak intensity of the fluorescent signal and consequently the NPs were directly labeled by the same FITC dye. In order not to drastically affect the NPs' surface (core Yb(III)-maghemite NPs for control and HSA-28P f-NPs), labeling was performed via a simple adsorption technique. HSA-28P and control core NPs (Yb(III)-maghemite NPs; 1.5 mL aqueous dispersion, Fe=1.45 mg/mL, a total Fe amount of 2.175 mg) were treated with 2.73 mg of FITC (fluorescein isothiocyanate, 7.01 μmol) in 5 mL of milliQ-purified H₂O and the mixture was shaken for 2 hours at room temperature (60° rotation angle, 80 rpm). Excess FITC was removed by centrifugation-mediated washings (3×10 mL ddH₂O) using an Amicon® Ultra-15 centrifugal filter device (100K) operated at 4,000 rpm (5 min), followed by a 2 cycles of gravitational centrifugation (4,000 rpm/min, 6 min, 15° C.). Cleaned and labeled NPs were finally dispersed in 4 mL of H₂O (Fe=0.157 mg/mL). For both HSA-28P f-NPs, 3 mL of each peptide-conjugated f-NP aqueous suspension (Fe=0.241 mg/mL, total Fe amount of 0.723 mg) were brought in contact with 4.71 mg FITC (12.1 μmol). Each mixture was then shaken overnight (200 rpm) at 14.5° C. and excess FITC was washed with milliQ-purified H₂O (4×10 mL) using centrifugation-NP precipitation (4 cycles, 4,000 rpm, 5 min, 15° C.). The resulting cleaned FITC-labeled fluorescent f-NPs were then re-dispersed in 2.5 mL of H₂O (Fe=0.0724 mg/mL).

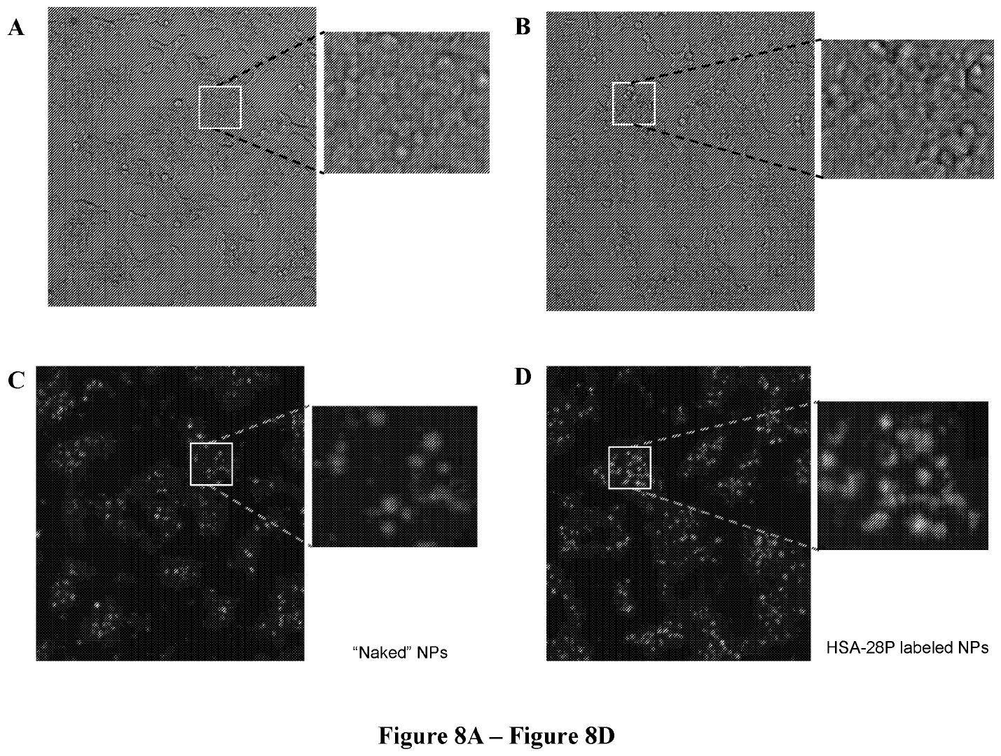

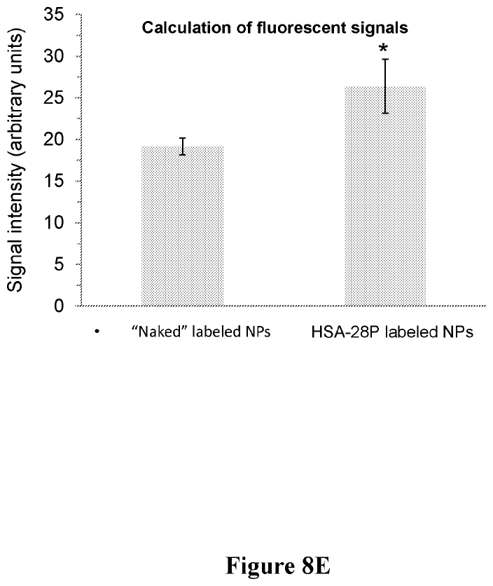

FITC-labeled HSA-28P f-NPs were added to INS-1E cells for 24 h, washed, and then assessed by fluorescent microscopy. As shown in FIG. 8A, the amount of cells that was not treated by labeled HSA-28P was significantly less than the amount of treated cells (FIG. 8B). A significant difference in the fluorescent signal was also obtained (see FIG. 8C and FIG. 8D). However, to rule out the possibility that the differences in the fluorescent signal are only related to the absolute obvious changes in the number of cells, zoom out pictures were taken (FIG. 8). These pictures show approximately identical amounts of confluent cells in both treatment groups, although with significant differences in the fluorescent signals. The fluorescent intensity of both treated and untreated cells is summarized in FIG. 8E. Importantly, the fluorescent signal is selectively associated with the cells and was not detected in the medium.

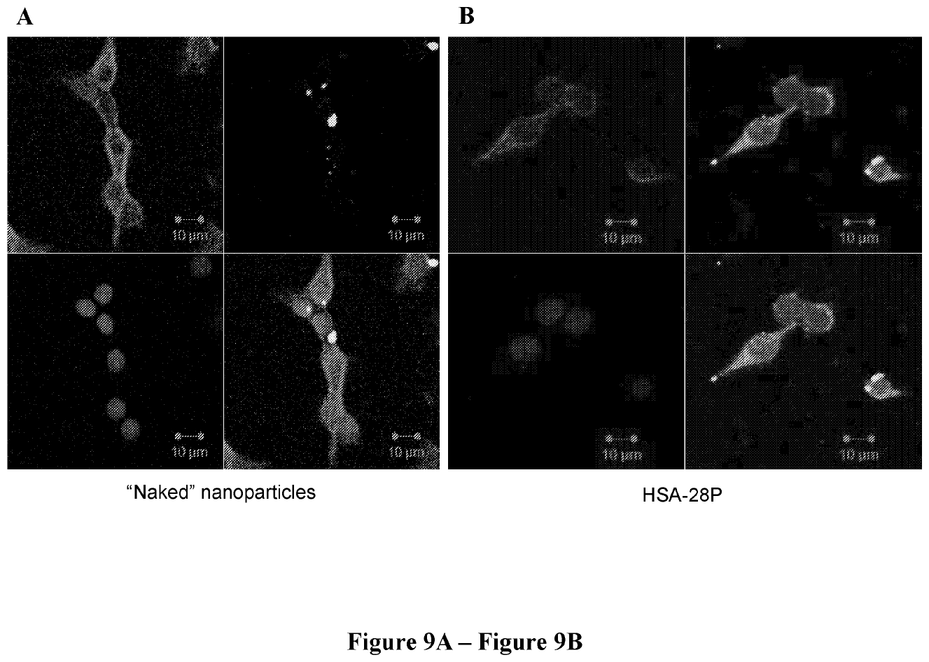

The plasma membrane of β-cells was hypothesized to be the primary target for NL-2 mimetics. Thus, the association of the NPs with the plasma membrane of INS-1E was investigated. To this end, INS-1E cells were grown on cover slips, and then incubated with HSA-28P (Fe concentration, 0.0035 mg/ml) for 24 h. After fixation, phalloidin staining was used to mark the plasma membrane and DAPI was applied to indicate the cells' nuclei. The resulting slides were subjected to confocal microscopy. As shown in FIG. 9, FITC-labeled HSA-28P f-NPs were localized solely on the plasma membrane, in contrast with cells treated by “naked” FITC-labeled NPs. Weak fluorescent signals were observed in “naked” FITC-labeled NP cells in comparison with strong signals in cells treated by HSA-28P. The data are presented in FIG. 8. These data indicate that HSA-28P interacts with the plasma membrane strongly and specifically and that a massive washing procedure (2 washes of live cells with medium and a total of 16 washes of fixed cells with PBS) did not affect the binding affinity of HSA-28P. Moreover, the control NPs, lacking specific NL-2 interactions, were easily washed out.

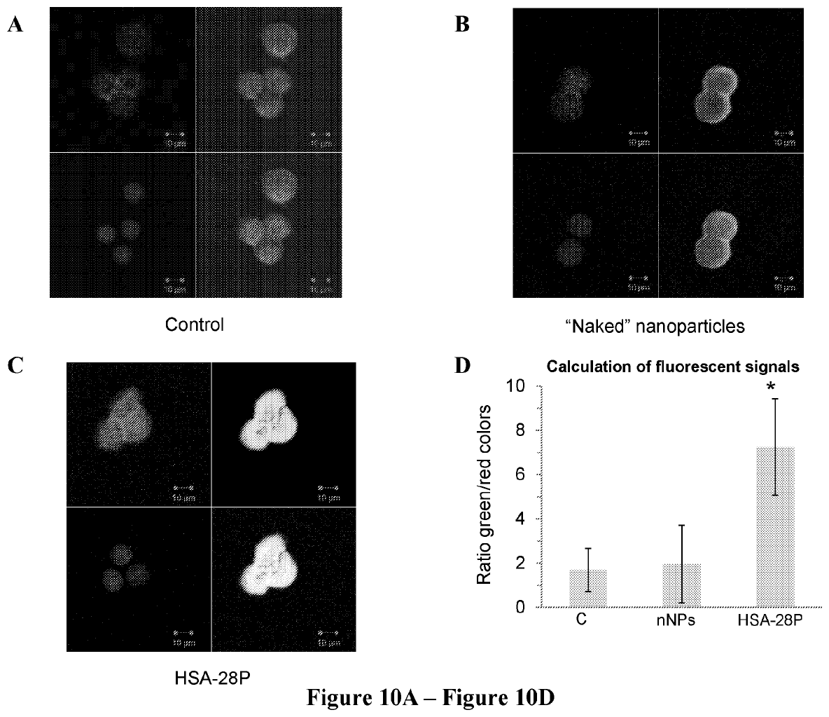

The positive effect of HSA-28P on the rate of insulin secretion and the insulin intracellular content was further demonstrated by an immunocytochemistry-based assay, namely, the determination of the intracellular accumulation of C-peptide. This small peptide links between two chains of insulin and assists in processing mature insulin inside the endoplasmic reticulum. Importantly, the ratio between C-peptide and insulin is equal in secretory granules of β-cells and both proteins are secreted simultaneously in blood circulation. Thus, the measurement of C-peptide is an indirect, but a very precise method for determining insulin synthesis and storage. In contrast, direct measurement of insulin can be problematic because, in addition to endogenous insulin that β-cells produce, the insulin from the serum of growth medium is also able to bind to the primary antibody. In contrast, the primary antibody against C-peptide is selective only to one specific species (in this case rat β-cells) and in the added serum C-peptide is not present. INS-1E cells were treated as described above. The plasma membrane and nuclei were marked similarly. Slides were exposed to a primary antibody against C-peptide and after incubation and substantial washing the secondary antibody (goat-anti-rabbit IgG labeled by ALEXA Fluor 488 nm) was added. The cells were tested by confocal microscopy. Cells treated by HSA-28P expressed significantly more C-peptide (approximately 3-fold) than did the control (“naked” NPs), which did not affect the basal level of the C-peptide (FIG. 10). The intensity of the C-peptide level was normalized by the intensity of the membrane signal. These data are consistent with the results that were obtained regarding the stimulatory effect of the compound on the insulin secretion and storage, as shown in FIG. 7A through FIG. 7C. Although the effect of HSA-28P on the insulin content was not as dramatic as its effect on the C-peptide accumulation, the positive correlation between these two effects suggests that the test compound indeed increases insulin production and secretion.

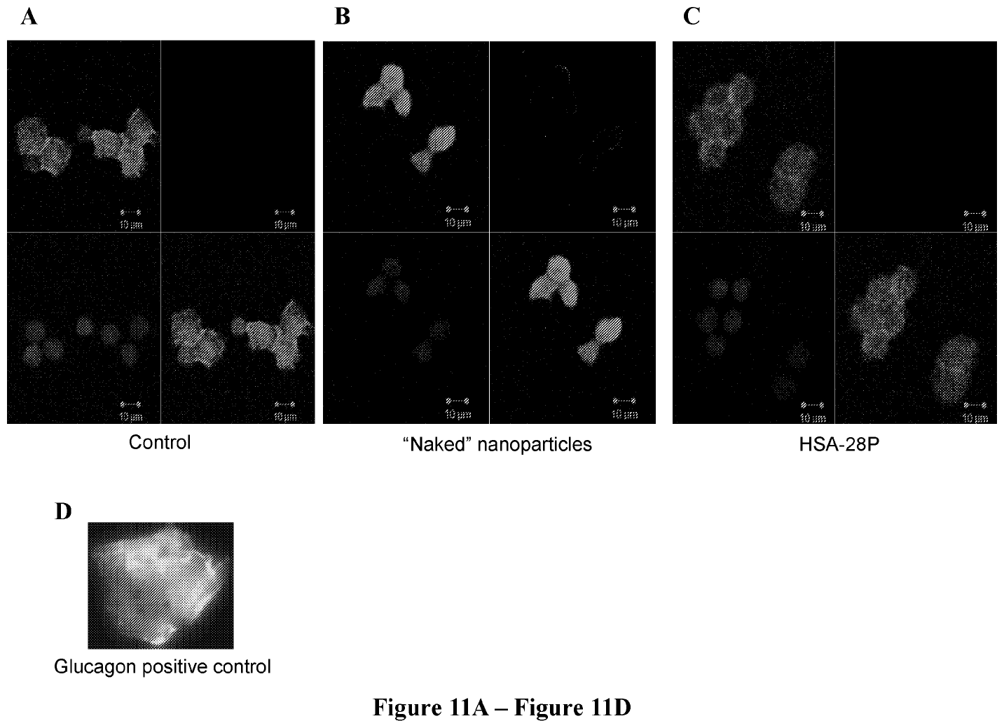

Sometimes, an increased proliferation rate of β-cell lines leads to a decrease in their differentiation level. This can be observed by abnormally elevated levels of other hormones, for example, the production and secretion of glucagon (the functional antagonist of insulin). Although the INS-1E cell line is the most commonly used in vitro model for studying β-cell functions), it is not an exclusively insulin-producing β-cell line, since basal levels of glucagon are still produced and secreted by these cells. Thus, the possible effect of HSA-28P on the level of glucagon was investigated (FIG. 11). Neither ‘naked” NPs nor HSA-28P affected the level of glucagon expression. These data support the conclusion that HSA-28P stimulates cell proliferation, insulin production, and secretion without significantly influencing the differentiated phenotype of the β-cells.

INS-1E is an immortalized cell line and may not entirely reflect the function of primary β-cells within Langerhans islets. Islets consist of a heterogeneous population of endocrine cells, including insulin-producing β-cells (approx. 65-70%), glucagon-secreting α-cells (20-25%), somatostatin-secreting β-cells, polypeptide (PP)-secreting cells, and β-cells producing the hormone ghrelin. The cellular and biochemical mechanisms by which glucose stimulates insulin secretion by pancreatic β-cells can be studied using islets isolated from rodents. Rat and mouse islets serve as an ideal source of insulin-producing tissue to study pancreatic β-cell function, and it is possible to obtain 300-600 islets/rat or 80-180 islets/mouse from a single pancreas. The method used to isolate pancreatic islets is based on the protocol originally developed by Lacy and Kostianovsky (1967) but with some modifications. It was noted that insulin secretion-affecting compounds could also be studied with isolated pancreatic islets from mouse islets β-cells. However, this method compromises the native environment of the β-cells and although it is possible to obtain highly purified cell cultures (α-cells and other islet cells can be easily separated from the β-cells), their relevance to true physiological conditions is questionable.

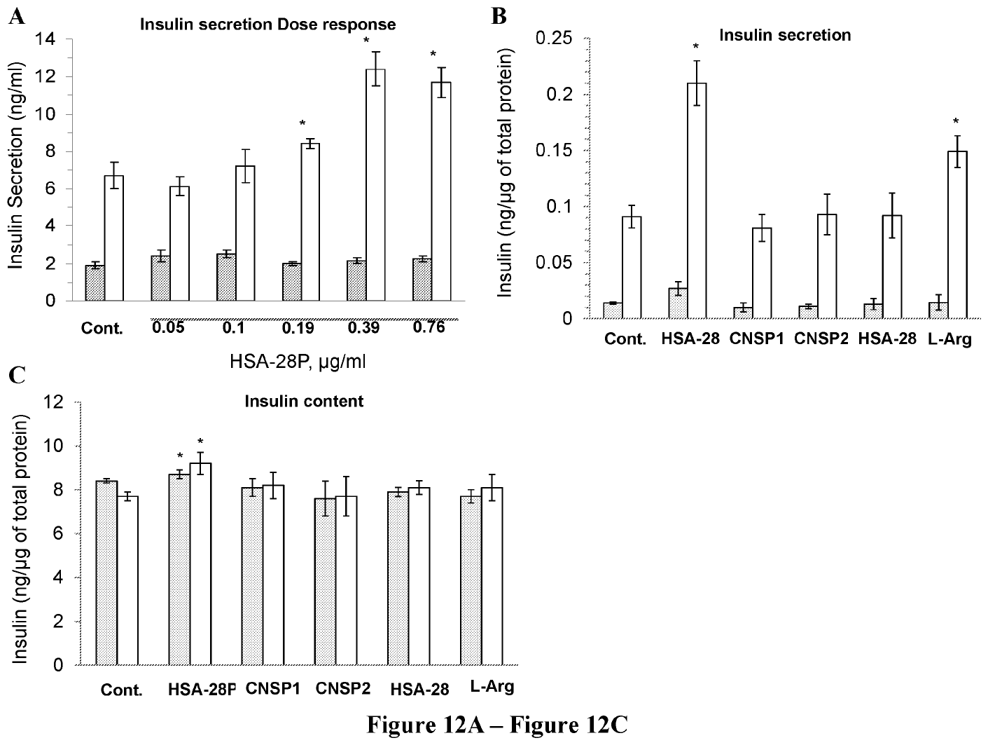

Therefore, the effect of HSA-28P on GSIS was also studied in freshly isolated mouse islets. It was found that HSA-28P significantly increases insulin secretion in a dose-dependent manner (FIG. 12A and FIG. 12B). The maximal effect was observed at a concentration of 0.39 μg/ml (around a twofold stimulatory effect). In addition, two types of NPs, which were covered (see the Supplemental material) by CNS active NL-1 peptides, were tested. Both NPs (CNSP1 covered by SEGNRWSNSTKGLFQRA (SEQ ID NO:2) sequences and CNSP2 covered by HSEGLFQRA (SEQ ID NO:3) sequences) were unable to increase the GSIS in isolated islets. In contrast, L-arginine, which served as a positive control, significantly raised GSIS in isolated mouse islets (FIG. 12B). The small but significant effect on the insulin content was obtained upon treatment of isolated mouse islets by HSA-28P using both tested concentrations (FIG. 12C).

In summary, the data herein demonstrates that the activation of the NL-2 pathway represents a novel strategy for regulating pancreatic β-cell numbers and functional maturity. Use of the neuron machinery present in β-cells in a “frozen” form due to their embryonal source for the secretion of mediators was never reported before. Starting with a computational work, a single NL-2-derived peptide was chosen for in vitro evaluation. Presenting multiple copies of this peptide on the surface of nanoparticles to β-cells increases insulin secretion, leads to the proliferation of insulin-containing β-cells, and protects cells against oxidative stress. The positive effect of HSA-28P was also obtained in isolated mouse islets, which makes the approach presented in this study physiologically relevant. Moreover, the use of the NL-2-based clusters may support the differentiation and maturation of human embryonic stem cells derived from pancreatic progenitors.

HSA-28D Biological Evaluation.

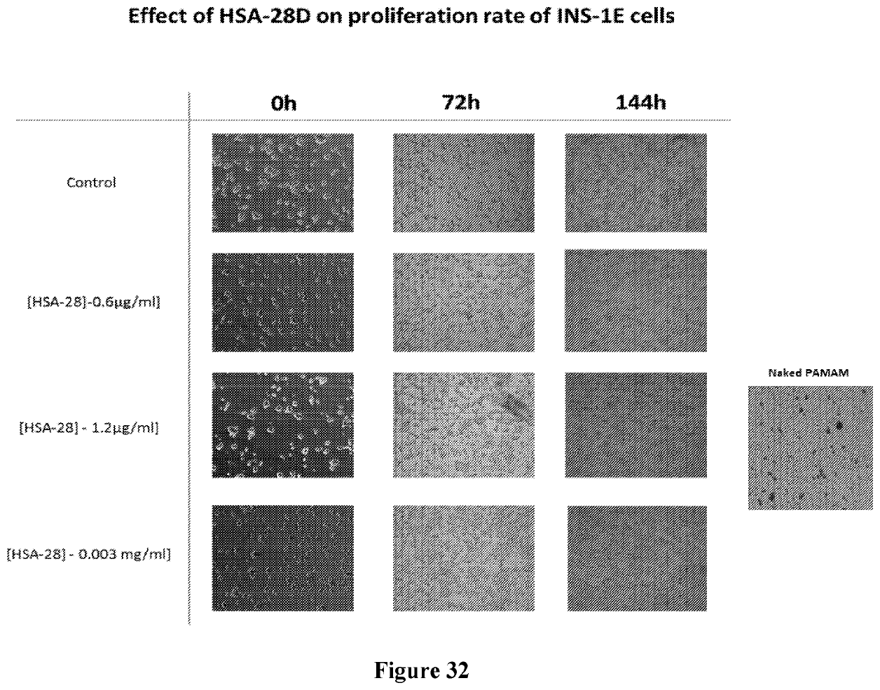

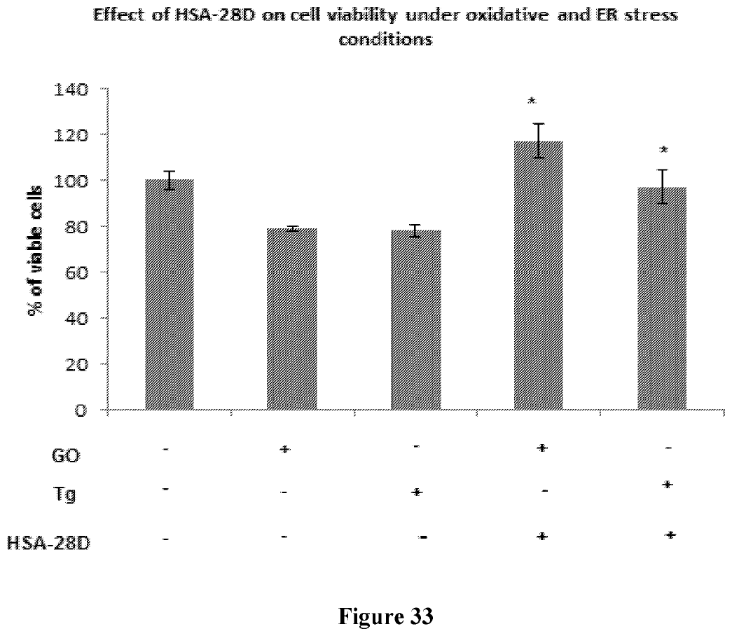

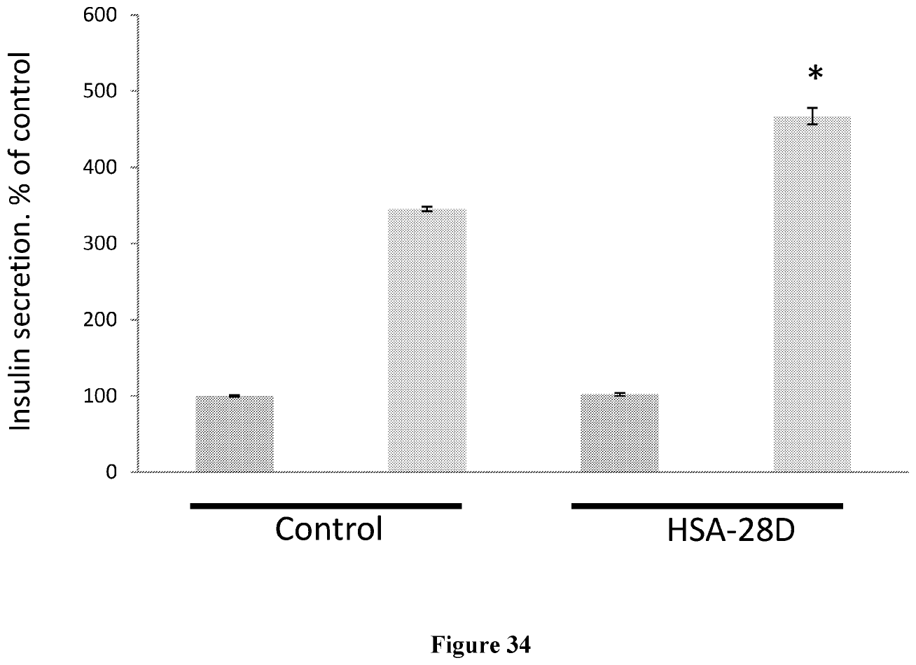

Coated by HSA-28 PAMAM dendrimer (generation 5) significantly increased the rate of cell proliferation (FIG. 32) after 72 and 144 hours. The effect of HSA-28D on cell viability under oxidative and ER stress conditions was also investigated. It is known that under diabetic conditions, oxidative stress and endoplasmic reticulum (ER) stress are induced in various tissues including β-cells. Two systems were used for generation of ER and oxidative stress conditions in vitro to mimic the diabetic conditions in vivo. First, thapsigargin (Tg) was used as a ER stress inducer. The toxin elevates intracellular level of calcium and this leads to ER stress. Second, glucose oxidase enzyme (GO) was used as an oxidative stress inducer. The enzyme catalyzes the oxidation of glucose in the medium to hydrogen peroxide and thus induces the oxidative stress. The cytoprotective effect of HSA-28D on cell viability under oxidative and ER stress conditions are presented in FIG. 33. The stimulatory effect of HSA-28D on the rate of the insulin secretion in high glucose medium in INS-1E cells is presented in FIG. 34.

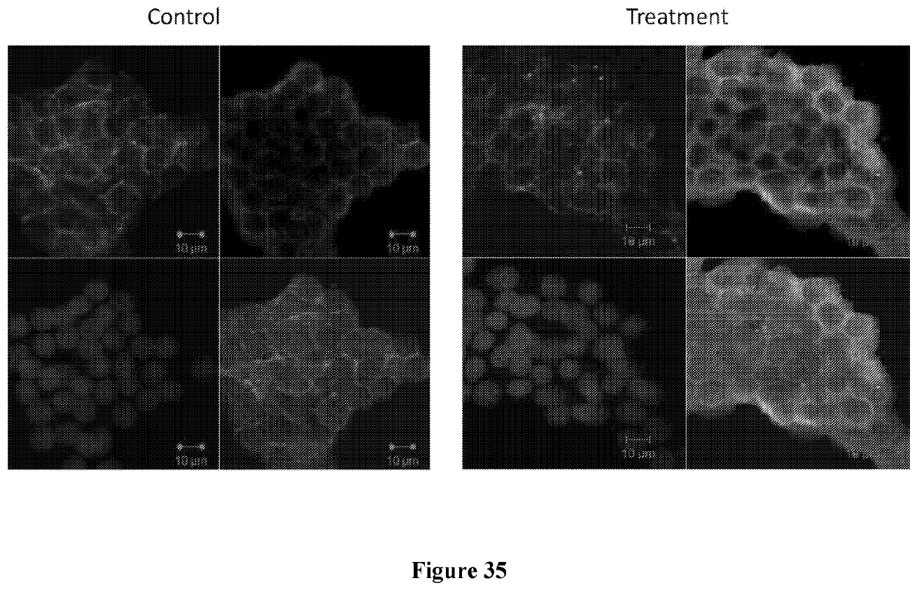

The positive effect of HSA-28D on the insulin intracellular content was reviled by a measurement of intracellular accumulation of C-peptide via an immunocytochemistry-based assay (FIG. 35). C-peptide is a small peptide that links between two chains of insulin in a proinsulin molecule and assists in processing mature insulin inside the endoplasmic reticulum. The ratio between C-peptide and insulin is equal in secretory granules of β-cells and both proteins are secreted simultaneously in blood circulation. Thus, the measurement of C-peptide is an indirect, but a very precise method for determining insulin synthesis and storage.

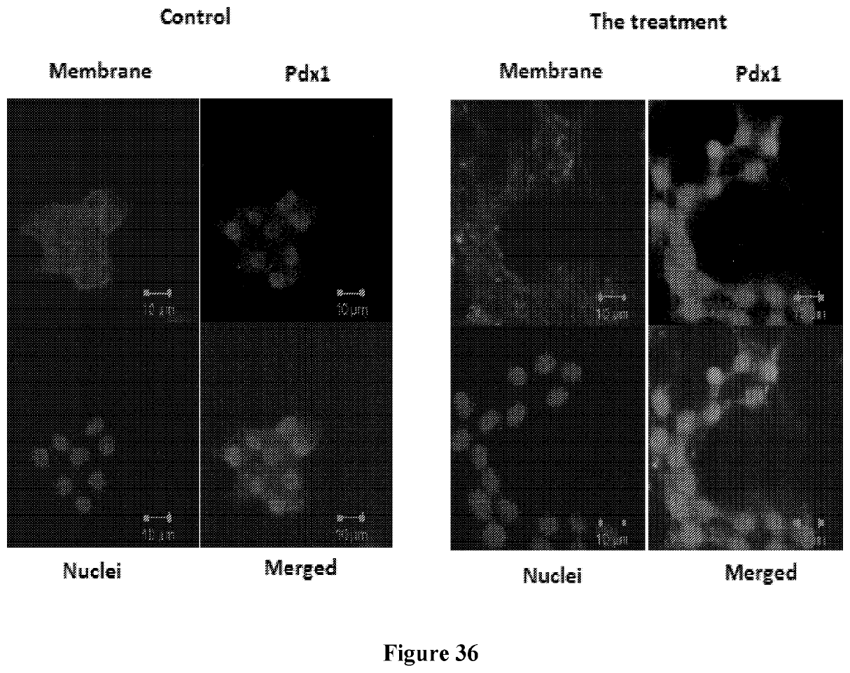

Pdx1 (Pancreatic and duodenal homeobox 1) is a transcription factor which necessary for pancreatic development and β-cell maturation. Diabetes type 2 is characterized by Pdx1-deficient (3 cells. Thus, the effect of HSA-28D on the intranuclear amount of Pdx-1 was investigated. Indeed, the compound significantly increased the level of Pdx-1 in INS-1E cells, as shown in FIG. 36.

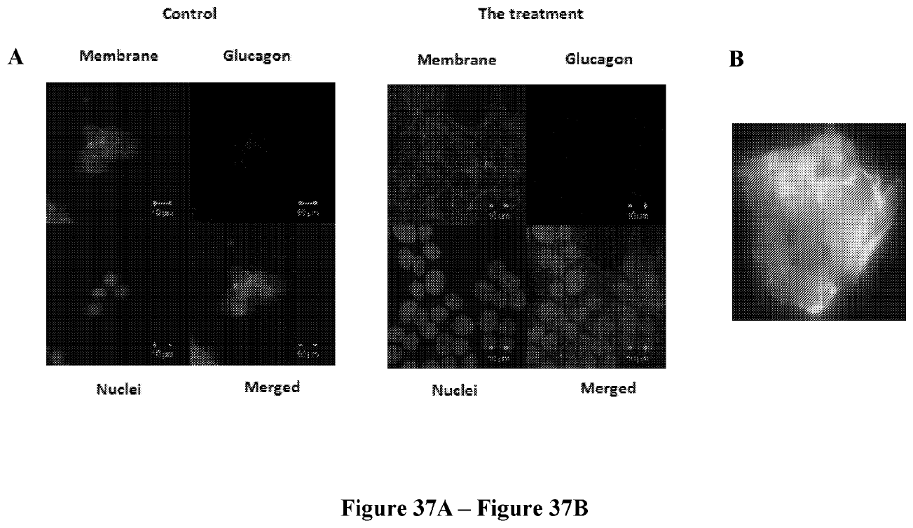

Increased proliferation rate of β-cells lines sometime leads to a decrease in their differentiation. Thus, abnormally elevated levels of other hormones might be observed, for example the production and secretion of glucagon. Glucagon is produced by alpha cells of the pancreas and raises the concentration of glucose in the blood. Its effect is opposite that of insulin, which lowers the glucose concentration. Therefore, the possible effect of HSA-28P on the level of glucagon in INS-1E cells was investigated by an immunocytochemistry assay. As shown in FIG. 37, HSA-28D did not induce the production of glucagon in INS-1E cells.

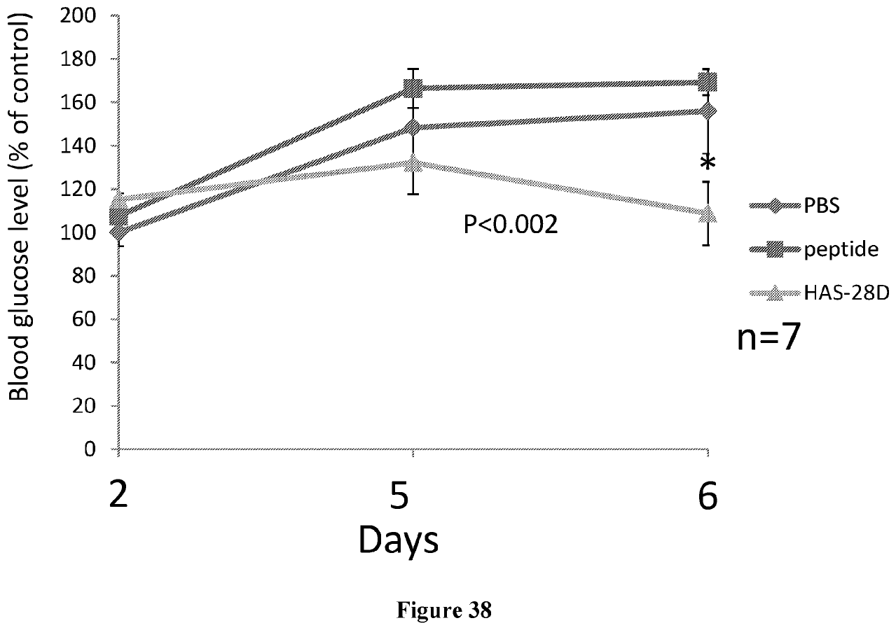

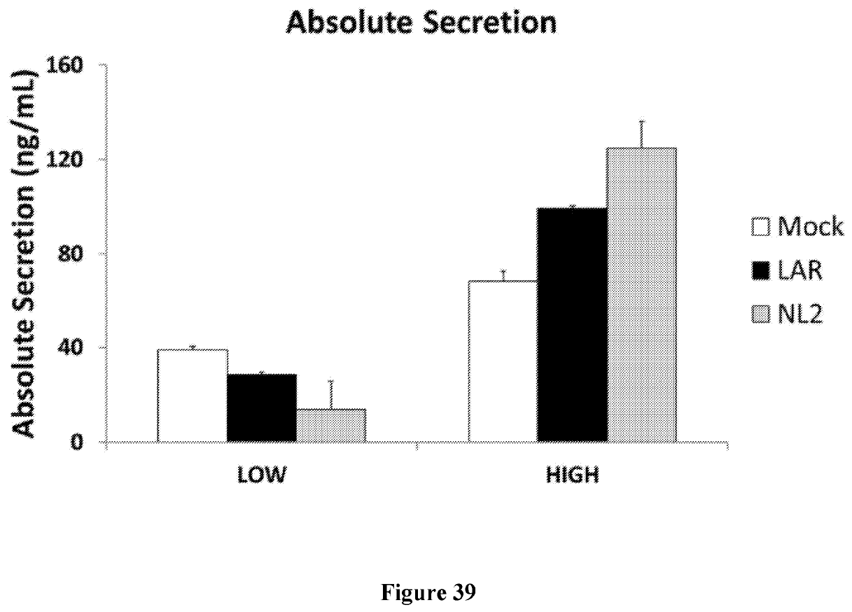

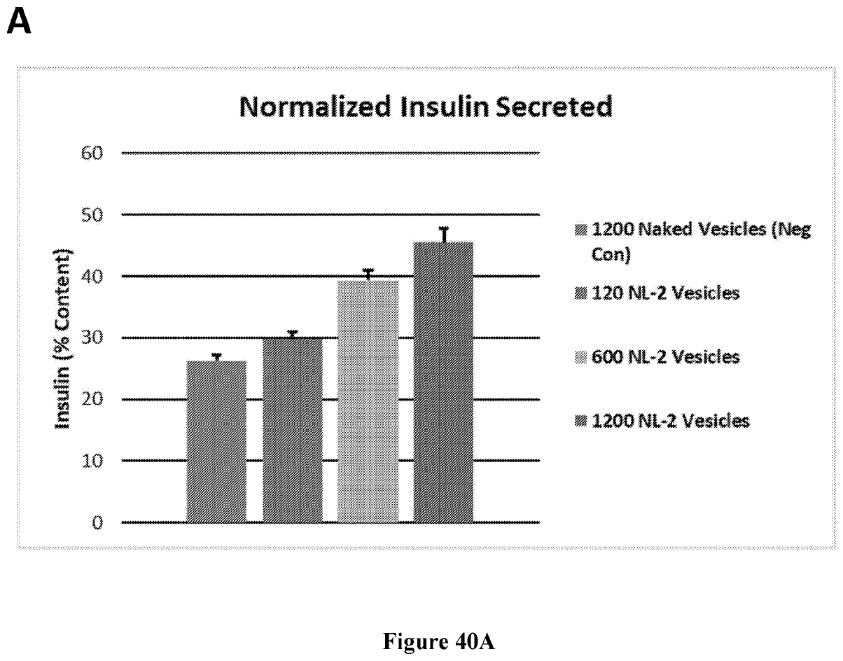

Finally, the antidiabetic effect of HAS-28D was evaluated in LDSTZ treated C57 black mice. In FIG. 38 shown that the compound significantly reduced the blood glucose level almost by 60%.

### Example 2: Pathways Through which Neuroligin-2 Promotes β-Cell Maturation and Insulin Secretion

It is hypothesized herein that neuroligin-2 function is triggered by the clustering of its extracellular domain and, in turn, by the clustering of its binding partner neurexin and likely other transcellular binding partners. It is further hypothesized that the intracellular domain of neuroligin-2 also influences insulin secretion through interactions with gephyrin and other cytoplasmic binding partners.

Neuroligin-2 is present on the β-cell surface and engages in trans-cellular interactions that influence insulin content and assembly of the insulin secretory machinery (Suckow et al., 2012, J Biol Chem 287:19816-26; Suckow et al., 2008, Endocrinol 149:6006-17). Indicative of its importance, neuroligin-2 was the 10th most abundant β-cell transcript identified in a study of human islet-specific plasma membrane proteins (Maffei et al., 2004, Endocrinol 145:4513-21). The objective of the studies presented herein is to understand how neuroligin-2 functions in the β-cell, including testing the role of clustering and the role of its intracellular domain. It is hypothesized herein that neuroligin-2 function is activated by its clustering on the cell surface and the resulting clustering of its trans-cellular binding partners. The cytoplasmic domain interacts with gephyrin and Epac2, key proteins involved in regulation of β-cell function; it is tested how the intracellular domain influences insulin secretion.

The extent of overlap between β-cells and neurons is such that mammalian β-cells, which develop from the embryonic endoderm, were once thought to be derived from the neural ectoderm (Le Douarin et al., 1998, Cell 53:169-71). Whole transcriptome analysis of the human β-cell drives home this point, revealing that differentiated β-cells are characterized by a neuron-like pattern of gene expression (Nica et al., 2013, Genome Res 23:1554-62). One striking resemblance is between the β-cell machinery for regulated secretion and the central nervous system (CNS) machinery for synaptic neurotransmission. Many of the important scaffolding, subplasmalemmal and synaptic vesicle proteins are also key components of the insulin secretory machinery (Abderrahmani et al., 2004, FEBS Lett 565:133-8; Drebach et al., 2001, Cell Mol Life Sci 58:94-116; Easom et al., 2000, Semin Cell Dev Biol 11:253-66; Atouf et al., 1997, J Biol Chem 272:1929-34).

A breakthrough in understanding how synaptogenesis (synapse formation) occurs came with the discovery of synaptic adhesion molecules that guide the formation of synapses. Like other synaptic adhesion molecules, they participate in protein-protein interactions across the synaptic cleft, helping to maintain the proximity of the pre- and post-synaptic densities. Uniquely, however, these molecules trigger synaptogenesis when brought into contact with neuronal processes (Craig et al., 2006, Trends Neurosci 29:8-20). These synapse-inducing proteins include the neuroligins, which are postsynaptic, and their major binding partners, the neurexins, which are found on the presynaptic membrane and likely nucleate the assembly of the submembrane secretory apparatus (Craig et al., 2007, Curr Opin Neurobiol 17:43-52). The neuroligins and neurexins were initially thought to be brain-specific.

The β-cells, are postulated to have inherited an inhibitory-synapse-like phenotype from their neural ancestor (Arntfield et al., 2011, BIoEssays 33:482-7). For example they express the vesicular GABA transporter, a specific marker of inhibitory synapses, and that microvesicle trafficking in the β-cells utilizes a mechanism important for inhibitory synaptic function (Chessler et al., 2002, Diabetes 51:1763-71; Suckow et al., 2006, J Mol Endocrinol 36:187-99; Suckow et al., 2010, Am J Physiol Endocrinol Metab 299:E23-32). Trans-cellular clustering interactions involving neuroligin-2 enhance maturation of the subplasmalemmal insulin secretory machinery and are necessary for its normal functioning (Suckow et al., 2012, J Biol Chem 287:19816-26). Further, the neuroligin binding partner neurexin is directly associated with the insulin secretory machinery and is important for insulin granule docking (Mosedale et al., 2012, J Biol Chem 287:6350-61). Only two trans-cellular protein interactions that play a direct role in insulin secretion have been identified. These are β-cell coupling by connexin36 channels (Ravier et al., 2005, Diabetes 54:1798-807; Kelly et al., 2011, Islets 3:41-7) and ephrin-A5-EphA5 interactions (Konstantinova et al., 2007, Cell 129:359-70). Alterations in E-cadherin and neural cell adhesion molecule expression affect insulin secretion, but it is not known whether the changes are attributable to effects on trans-cellular interactions (Jaques et al., 2008, Endocrinol 149:2494-505). The results presented herein implicate neuroligin-2 and neurexin as key proteins mediating the beneficial effects of β-cell-β-cell contact

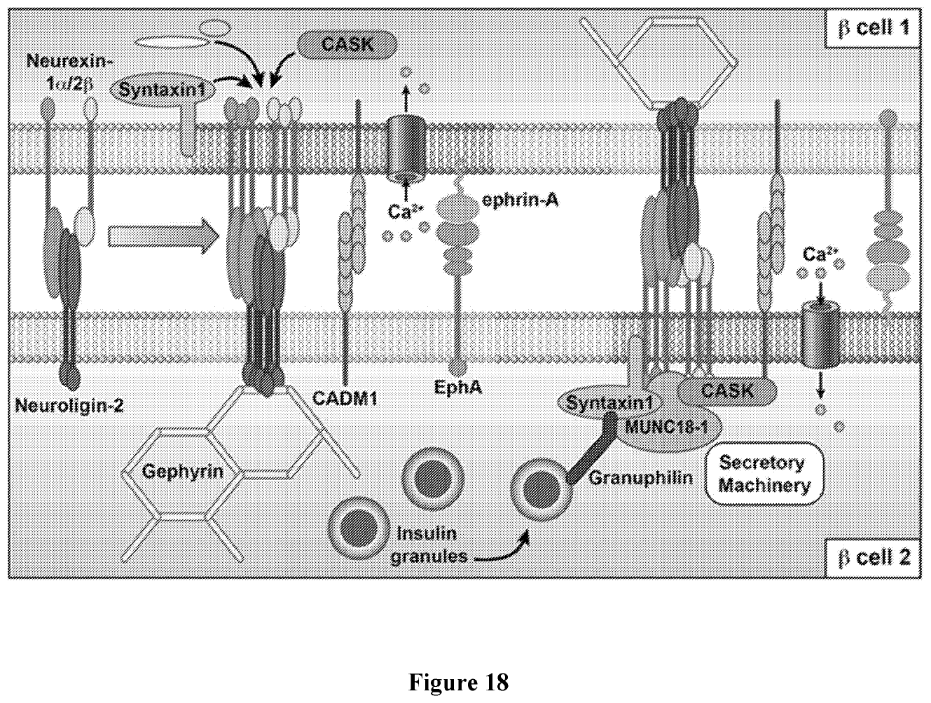

FIG. 18 depicts the working model. It shows the synaptogenic proteins that have been confirmed to be expressed by β-cells thus far—neuroligin-2, neurexin (Suckow et al., 2008, Endocrinol 149:6006-17) and CADM (manuscript under revision)—and, as described by a different group, Eph/Ephrin (Konstantinova et al., 2007, Cell 129:359-70. The proteins are shown localized to hypothesized pre- and post-synaptic-like plasma membrane domains. While not wishing to be bound to any particular theory, the presynaptic-like domains (red in FIG. 18) are, may be the previously-described, β-cell plasma membrane exocytic microdomains (Rutter et al., 2006, Cell Calcium 40:539-51).

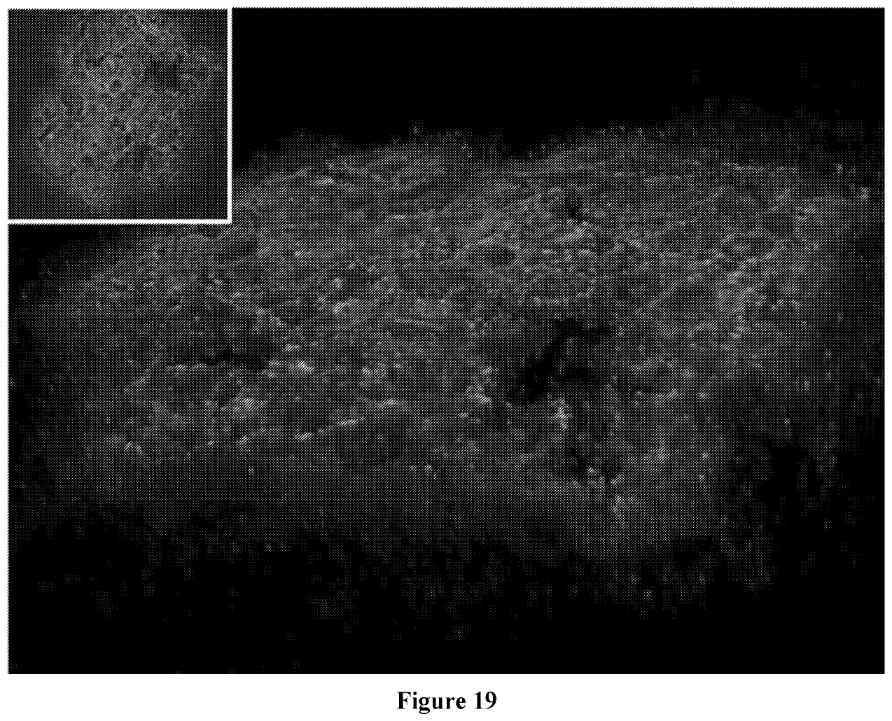

FIG. 19 shows that neurexin, which is an integral part of the insulin secretory complex, localizes to discrete sites on the β-cell surface. Members of the submembrane insulin secretory complex including CASK, syntaxin-1 and granuphilin are, as depicted in FIG. 18, associated—directly or indirectly—with neurexin (Mosedale et al., 2012, J Biol Chem 287:6350-61). Consistent with this model (FIG. 18), CASK and MUNC18-1 colocalize at sites of β-cell-to-β-cell contact, and whole-islet imaging reveals insulin secretory microdomains to be aligned along sites where β-cell edges come into contact (Tomas et al., 2008, Traffic; Geron et al., 2015, Cell Reports 10:317-25)

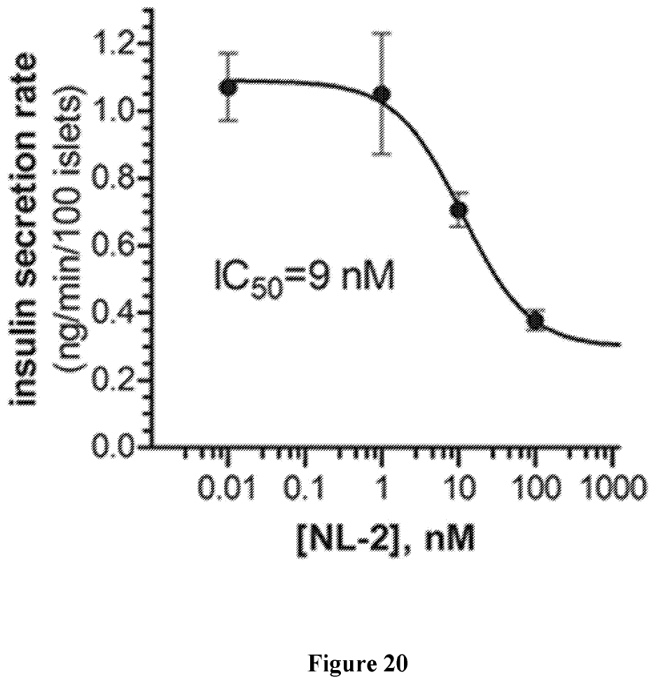

When clustered, the extracellular domain of neuroligin induces formation of presynaptic active zones (sites of neurotransmitter secretion) upon contact with neural processes (Scheiffele et al., 2000, Cell 101:657-69; Dean et al., 2003, 6:708-16; Graf et al., 2004, Cell 119:1013-26; Song et al., 1999, PNAS 96:1100-5). Similarly, clustered neurexins trigger postsynaptic density formation (Dean et al., 2003, 6:708-16; Graf et al., 2004, Cell 119:1013-26). Because neuroligins and neurexins bind across the synaptic cleft, clustering of neuroligin induces clustering of bound neurexin and vice-versa. Neuroligin-2 function in β-cells, as depicted in FIG. 18, is similarly dependent on clustering. Neuroligin-2 presented on the surface of HEK293 cells increased insulin secretion when brought into contact with β-cells (Suckow et al., 2012, J Biol Chem 287:19816-26). In contrast, soluble neuroligin-2—by interfering with endogenous neuroligin interactions and clustering—impaired secretion (FIG. 20).

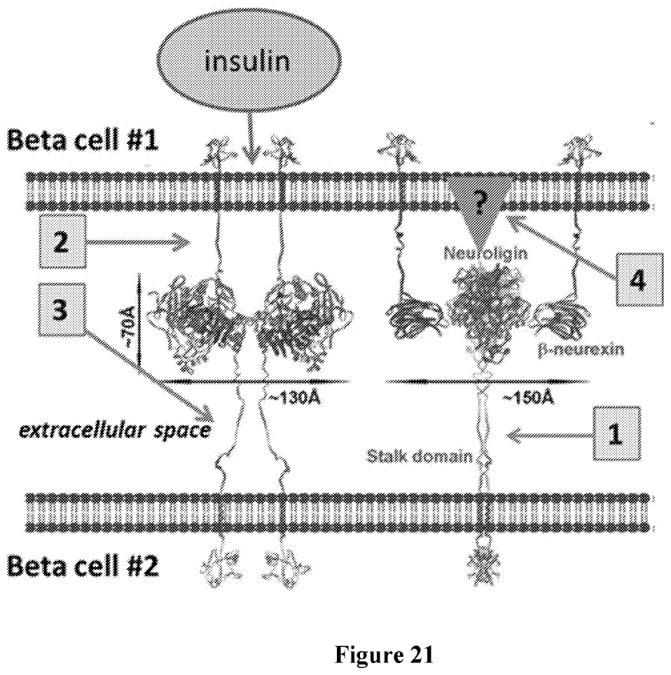

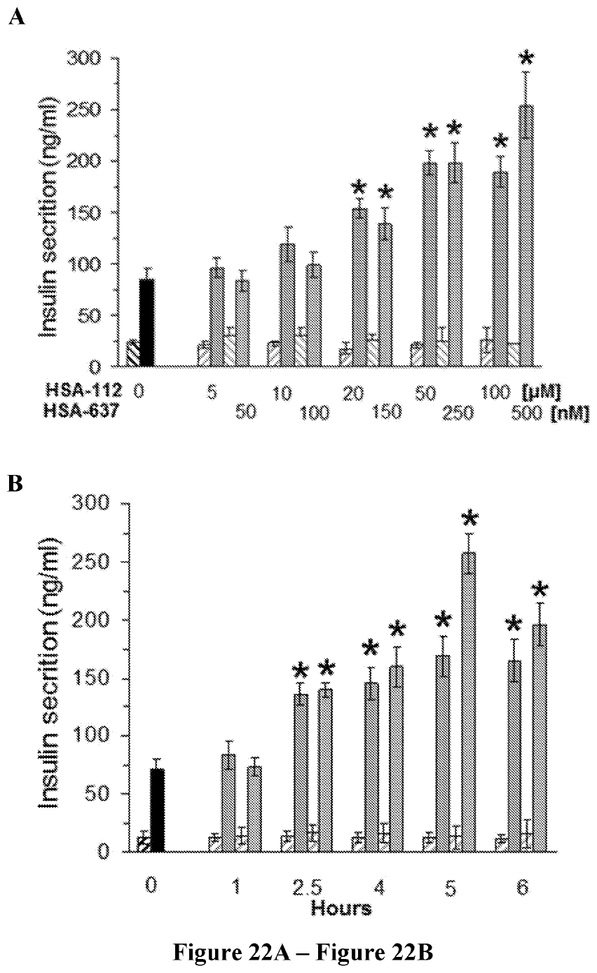

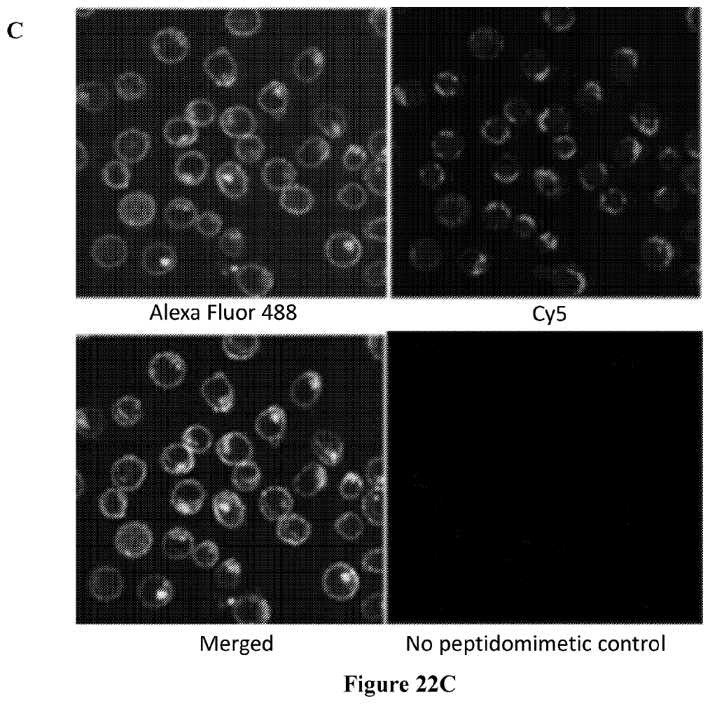

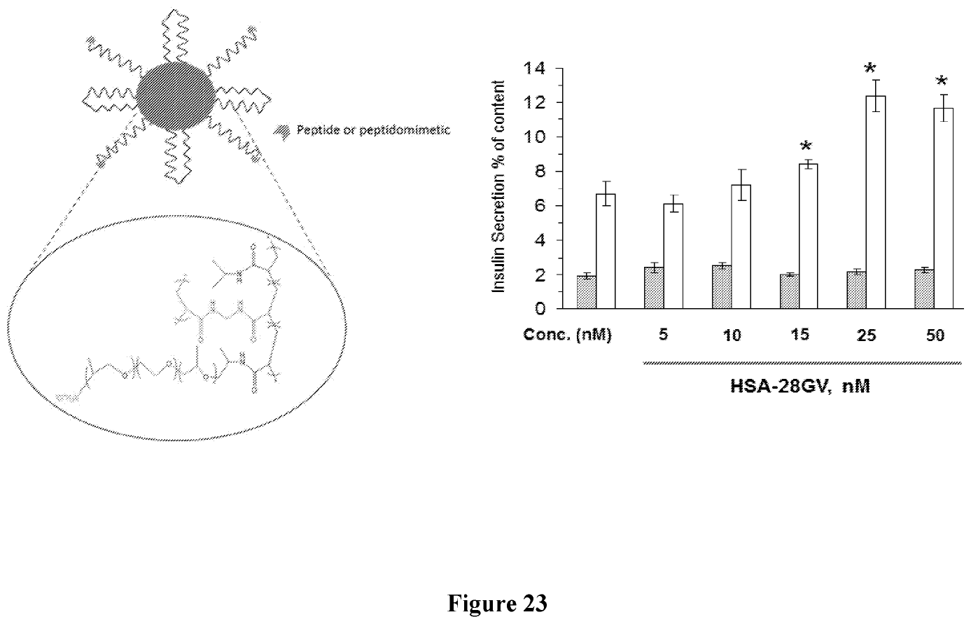

Although not wishing to be bound to any particular theory, this postulated mechanism—neuroligin-2 enhancing insulin secretory function upon clustering—presents an opportunity for exploiting neuroligin-2 and/or its binding partners as therapeutic targets (FIG. 21). To further test the mechanistic importance of clustering and simultaneously evaluate this therapeutic approach, agents that mimic clustered neuroligin are used. Peptidomimetic molecules emulating neuroligin-2's neurexin binding domain have been synthesized. Monomers and dimers of the peptidomimetic reagent (compound “HSA-28”) had no effect on INS-1 (3 cells (not shown). However, HSA-28 dimers clustered on a polyethylene glycol backbone (compound “HSA-112”) yielded increased insulin secretion in a dose-dependent manner (FIG. 22A). More extensive clustering of HSA-28 was achieved by using it to coat a nanoparticle (compound “HSA-637”). This reagent also enhanced insulin secretion (FIG. 22B). An additional compound made using a highly biocompatible nanoparticle (Byk et al., 2012, J Pept Sci 18:S1), was synthesized and tested with rat islets with similar results (FIG. 23). Additionally, clustered, recombinant neuroligin-2 enhanced insulin secretion from human islet β-cells (discussed below). These data are consistent with the explanation that clustering is essential for neuroligin-2 function and that this mechanism is a promising therapeutic target.

Does Clustering of Neurexin by Neuroligin Trigger Maturation and Enhanced Function of β-Cells? is this Pathway a Promising Therapeutic Target?

To further test whether clustered neuroligin-2 enhances β-cell function and functional maturation through clustering interactions with neurexin, the peptidomimetic-based reagents described above—which mimic specifically the neurexin binding site—are tested against primary islet cells, including human islet cells. Dissociated and intact rat and human islets are incubated with varying concentrations of clustered peptidomimetic agent HSA-112 and nanoparticle HSA-637 or HSA-G28V. It is then be determined if the reagents increase stimulated secretion, suppress basal insulin secretion and/or increase punctateness (a marker of degree of assembly) of the secretory protein assemblies. Punctateness is determined by immunofluorescent staining of syntaxin-1 followed by computer image analysis of the intensity of stained punctae. Functional maturation of β-cells is particularly evident during transition from very low fetal or neonatal levels of GSIS to normal (mature) levels (Navarro-Tableros et al., 2007, Am J Physiol 292:E1018-29). MafA is a key driver of such maturation, and it, pdx1 and urocortin-3 are markers of the more mature state (Aguayo-Mazzucato et al., 2011, Diabetologia 54:583-93; Blum et al., 2012, Nat Biotechnol 261-4). qPCR analysis is used to determine whether MafA, pdx1 and urocortin-3 transcript levels increase in response to treatment with the neuroligin-2-mimetic reagents

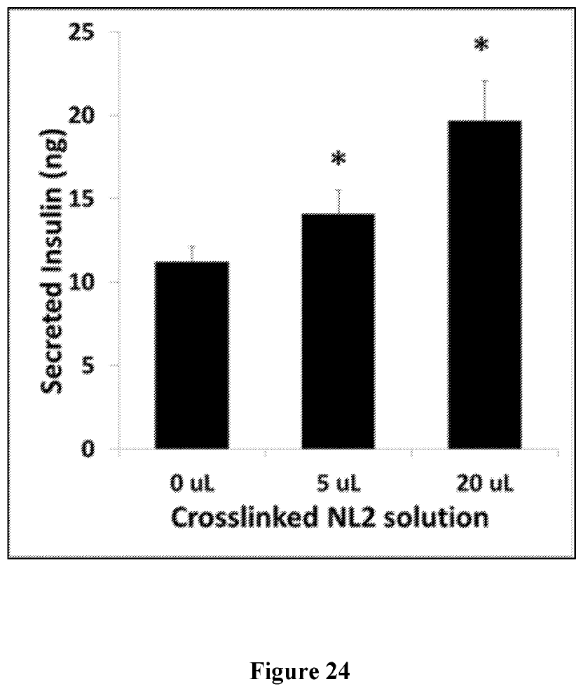

A recombinant protein with the extracellular domain of neuroligin-2 fused to an IgG Fc domain (rNL2ED-Fc) has been made. In contrast to the peptidomimetic agents, which are modeled on the neurexin binding site, this protein should be able to interact with all extracellular neuroligin-2 binding partners. Chemically cross-linked clusters incorporating this protein are made. Clustered complexes that sufficiently mimic endogenous clustered neuroligin-2 promote β-cell maturation and function to a greater degree than the peptidomimetic-based reagents. FIG. 24 shows results using rNL2-ED cross-linked using sulfo-N-succinimidyl 4-maleimidobutyrate sodium salt into a mixture of two complexes of 1121 kDa and 17320 kDa. Clustered neuroligin-2 enhanced insulin secretion by human β-cells in a dose-dependent manner. In contrast, rNL2ED monomers had no effect (not shown).

The additional cross-linked protein complexes and immunocomplexes that have been or are being generated are tested using INS-1 cells and human and rat islets. The clustered neuroligin-2 compound with the greatest effect on GSIS is used to show that exposure to clustered neuroligin-2 enhances maturation of the secretory machinery (as measured by punctateness of syntaxin-1), GSIS, and expression of mafA, pdx1 and urocortin-3. Using both the peptide reagents and neuroligin-2 extracellular domain in these experiments allow determination and differentiation of neurexin-dependent effects (peptide reagents) versus effects on maturation and secretion of interaction with the entire extracellular domain.

What is the Role of the Cytoplasmic Domain of Neuroligin-2 in β-Cell Function and does it Influence Insulin Secretion Via its Binding Partner Epac-2A?

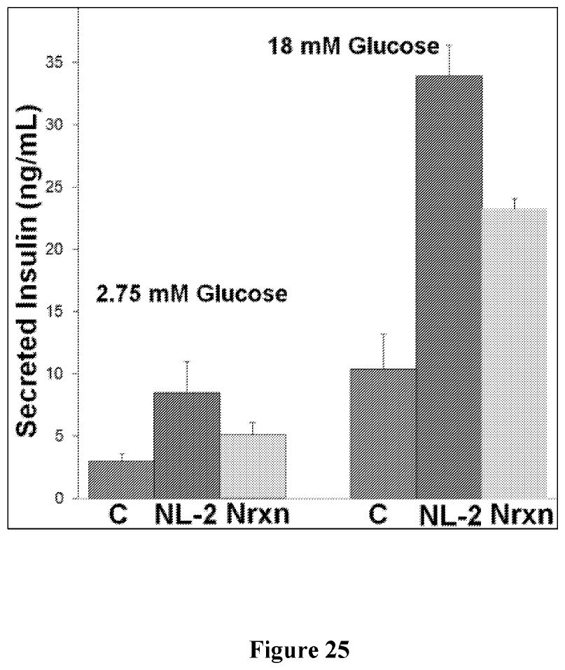

Coculture of β-cells with neurexin-1-expressing HEK293 cells, like neuroligin coculture (Mosedale et al., 2012, J Biol Chem 287:6350-61), increases insulin secretion (FIG. 25). This suggests that just an neurexin clustering by neuroligin enhances β-cell function, the converse is also likely true. In the model described herein (FIG. 18), neuroligin does not directly interact with the insulin secretory machinery. It is hypothesized that the cytoplasmic domain of neuroligin influences insulin secretion via its known binding partners Epac2 and gephyrin.

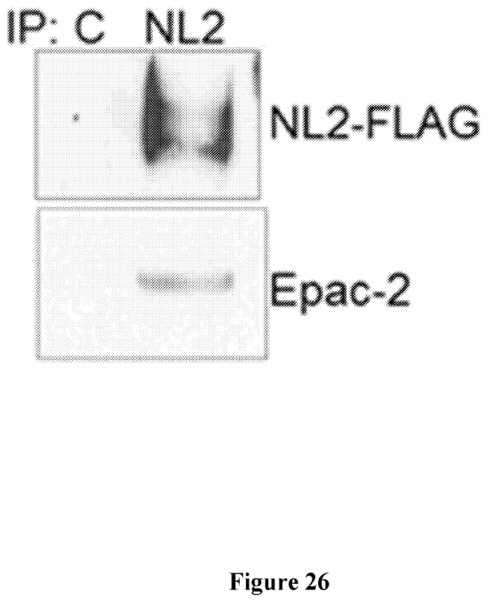

Epac-2A binds to the neuroligin-2 intracellular domain (Wollfrey et al., 2009, Nat Neurosci 12:1275-84). This previous finding was confirmed in β-cells (FIG. 26). Epac-2A plays a key role in the promotion of stimulated insulin secretion and its enhancement by GLP-1 (Holz et al., 2004, Diabetes 53:5-13; Song et al., 2013, Diabetes 2:2796-807). Two recent findings underscore the potential importance of this interaction with neuroligin-2 in β-cells. First, neuroligin binding enhances Epac-2A activity; second, neuroligin binding causes Epac-2A to localize to the plasma membrane (Wollfrey et al., 2009, Nat Neurosci 12:1275-84). Translocation of Epac-2A to the plasma membrane triggered by Ca²⁺ influxes and cAMP is integral to the β-cell's response to glucose stimulation (Idevall-Hagren, 2013, Sci Signal 6:ra29 1-11). siRNA-treated INS-1 cells and the islets from neuroligin-2 knockout mice are used to assess whether loss of neuroligin-2 expression impairs the ability of Epac-2A to localize to the plasma membrane. Differences in Epac-2 localization are analyzed by immunofluorescent staining and confocal microscopy. Epac-2 localization is analyzed under both basal and glucose-stimulated conditions. Co-immunoprecipitation is used to determine whether glucose stimulation of INS-1 cells increases Epac-2A binding to neuroligin-2. Epac-2A is immunoprecipitated from INS-1 cells under basal and glucose-stimulated conditions and levels of co-precipitated neuroligin-2 assessed by western blotting. There will be a greater degree of co-precipitation from cells with stimulated insulin secretion and that loss of neuroligin-2 will impair Epac-2 membrane localization. To evaluate the effect of neuroligin-2 on Epac-2A function INS-1 cells are treated with silenced neuroligin-2 expression and also islets from neuroligin-2 knockout mice with 8-CPT (8-4-Chlorophenylthio-adenosine-3′,5′-cyclic monophosphate), a selective Epac activator (Bos et al., 2006, Trends Biochem Sci 32:680-6; Imagawa et al., 1996, Res Mommun Mol Pathol Pharmacol 92:43-52). Insulin secretion is measured after treatment with 8-CPT or vehicle to assess whether lack of neuroligin-2 attenuates 8-CPT's stimulation of insulin secretion.

Does Neuroligin-2 Drive Gephyrin Clustering in the β-Cell and does the Neuroligin-2 Intracellular Domain Influence Insulin Through Gephyrin Interactions?

The model in FIG. 18 incorporates the known role of neuroligin-2 in nucleating the assembly of the post-synaptic submembrane protein scaffolding. This scaffolding is formed by the inhibitory post-synaptic protein gephyrin (Tyagarajan et al., 2014, Nat Rev 15:141-56). Gephyrin is of particular interest because of its global effects on synapse formation and function and its function as signaling hub, mediating of a number intracellular signaling pathways. Gephyrin mRNA is also a major target of a microRNA regulatory system operative in the β-cell—involving miR-375 and miR-184—that plays an essential role the control of islet cell mass and β-cell function (Poy et al., 2009, PNAS 106:5813-8; Tattikota et al., 2014, Cell Metab 19:122-34). Accordingly, knockdown of gephyrin in β-cells decreases insulin secretion (Tattikota et al., 2013, Mol Cell Proteomics 12:1214-25). The punctateness of gephyrin is assayed in the same way the punctateness of syntaxin is assayed to show that coculture with neurexin (as in FIG. 25) enhances gephyrin assembly and thus its punctateness. The neuroligin-2 constructs used carry epitope tags in their extracellular domains (HA or FLAG). To directly test whether neuroligin-2 clustering enhances insulin secretion and gephyrin clustering, neuroligin-2 is transfected into INS-1 cells or dissociated islet cells and clustering is induced using antibodies targeting the extracellular epitope tag (Graf et al., 2006, J Neurosci 26:4256-65; Mah et al., 2010, J Neurosci 30:5559-68. To test whether gephyrin mediates neuroligin function, it is determined whether siRNA silencing of gephyrin attenuates the effect on insulin secretion seen in coculture with neurexin-expressing HEK293 cells (FIG. 25). Transfection of neuroligin-2 into INS-1 cells and dissociated rat islet cells increases insulin secretion. This experiment is repeated comparing a neuroligin-2 construct lacking the known gephyrin binding site with wild-type neuroligin-2 to determine whether impairment of gephyrin binding prevents the increase in insulin secretion.

Does Clustering of Neuroligin-2 Affect Pharmacologically-Induced, Non-VDCC-Mediated Calcium Triggering of Insulin Secretion?

The presence of neurexin in secretory microdomains is essential for voltage-dependent calcium channel (VDCC) function (Dudanova et al., 2006, J Neurosci 26:10599-613). It is hypothesized herein that extracellular neuroligin interactions increase insulin secretion by enhancing secretory microdomain formation and—via neurexin—VDCC function and localization to the exocytic microdomains (FIG. 18). If the enhancement of insulin secretion by neuroligin is due to effects on VDCC function and/or VDCC localization at the secretory microdomains, pharmacologic treatments to increase cytoplasmic Ca²⁺ in a manner that bypasses the VDCCs should completely or partially reverse neuroligin-driven differences in stimulated insulin secretion (Rutter et al., 2006, Cell Calcium 40:539-51). INS-1 cells are cultured in 3 mM glucose and treated with clustered recombinant neuroligin (as were human islet cells in FIG. 24). They are then treated with the calcium ionophore A21387 (10 μM) and, separately, with thapsigargin, an agent that blocks Ca²⁺ uptake into the endoplasmic reticulum (5 μM). These treatments raise intracellular Ca²⁺ concentrations and thereby trigger insulin secretion. Since these treatments bypass VDCCs and fail to induce locally high Ca²⁺ concentrations at the secretory microdomains, they will eliminate or attenuate the increased insulin secretion induced by clustered neuroligin-2. Potassium-induced insulin secretion is analyzed to test that neuroligin-2 effects are due to effects on the exocytic machinery rather than the glucose-sensing mechanism. Neuroligin-treated cells still exhibit enhanced insulin secretion after potassium treatment, which directly depolarizes β-cells, bypassing the glucose sensing mechanism.

Is Neuroligin-2 Function Necessary for Voltage-Dependent Calcium Channel (VDCC) Function in β-Cells?

It is hypothesized that effects on VDCC function are another cellular mechanism whereby neuroligin-2 activity underlies normal β-cell function.

Clustering of neuroligin-2 and its binding partners enhances β-cell function. Further, neuroligin-2 plays a significant role in the function and localization of its cytoplasmic binding partner Epac-2, clustering of neuroligin-2 drives assembly of the gephyrin scaffolding and impairment of gephyrin-neuroligin binding attenuates the effect of neuroligin on insulin secretion.

### Example 3: The Role of Neuroligin-2 in Islet Development, in the Establishment of β-Cell Mass and Functional Maturation, and in Glucose Homeostasis in Mice

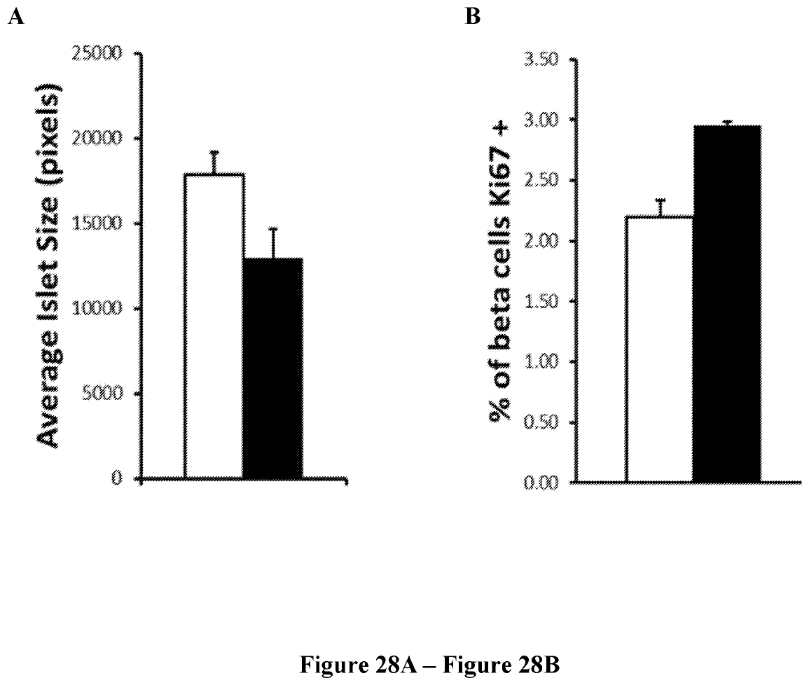

It is hypothesized herein that neuroligin influences islet size and number and is necessary for the attainment of normal β-cell mass and for normal glucose homeostasis in vivo. Results from a global neuroligin-2 knockout mouse demonstrate that neuroligin-2 deficiency has effects on β-cell function, pancreatic islet phenotype and number and, in the neonatal period, on β-cell proliferation (FIG. 28). Because the global knockout affects brain function, weight and activity levels, it is not a satisfactory model system for assessing effects on whole-body glucose homeostasis. A β-cell-specific knockout model is employed to test the effect of neuroligin-2 deficiency on β-cell function in vivo.

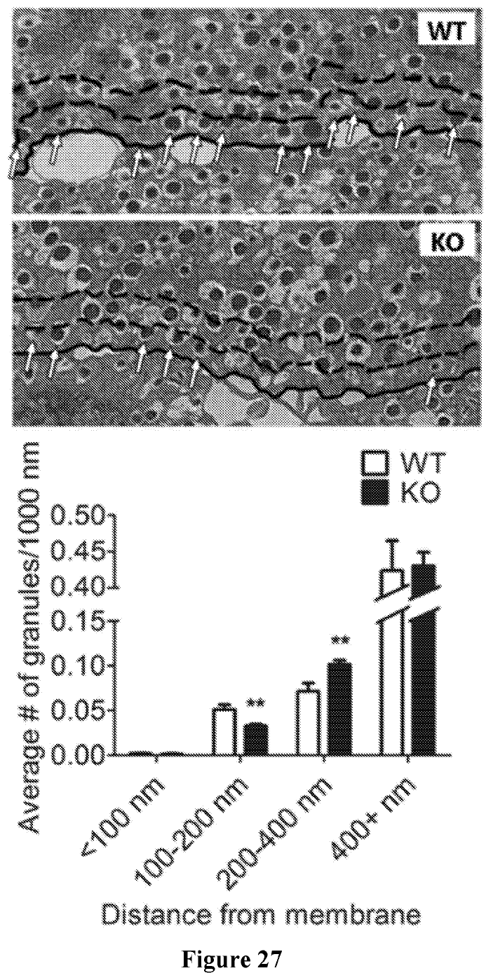

Islets from mice with global neuroligin-2 knockout exhibited enhanced insulin secretion after normalization to cellular insulin content, perhaps indicative of an increased efficiency of insulin secretion (Zhang et al., 2013, PLoS One 8:e65711). Islet insulin content, however, was markedly decreased, so, absolute secretion—in contrast to normalized secretion—was lower in knockout mice. Knockout mice exhibited decreased neurexin expression. Loss of neurexin impairs insulin granule docking in a manner that, like knockout of granuphilin and a number of other docking proteins, leads to increased insulin secretion (Mosedale et al., 2012, J Biol Chem 287:6350-61; Kasai et al., 2008, Traffic 9:1191-203). As seen in FIG. 27, docking was indeed decreased in the mutant mice. Islets in the mutant mice were also smaller and fewer in number, and pancreatic insulin content and β-cell area were markedly decreased. Subsequently it was found that neonatal global knockout mice also have smaller islets, but increased β-cell proliferation (ki67 staining; FIG. 27). These data indicate that neuroligin-2 affects β-cell development early on. To elucidate the role of neuroligin-2 in islet development and function, this analysis is repeated in pancreases from β-cell-specific knockout mice.

What is the Effect of β-Cell-Specific Loss of Neuroligin-2 on Islet Development, β-Cell Mass and Pancreatic Insulin Content?

To test the role of neuroligin-2 in islet development, β-cell-specific, conditional knockout mice are used. Mice with LoxP sites flanking neuroligin-2 exons 2-5 (B6; SJL-^{Nlgn2tm1.1Sud}/J; homozygotes are designated Nlgn2fl/fl) are bred with mice constitutively expressing Cre recombinase downstream of a mouse insulin promoter (B6[Cg]-Ins1^{tm1.1(cre)Thor/J}; designated here Ins1-Cre; does not encode human growth hormone). Use of a mouse insulin promoter to drive Cre expression in this strain avoids brain and other non-β-cell Cre expression (Tamarina et al., 2014, Islets 6:e2685; Wicksteed et al., 2010, Diabetes 59:3090-8; Thorens et al., 2015, Diabetologia 58:558-65). This tests the hypotheses that neuroligin-2, in parallel to its role in the synapse, helps to drive β-cell differentiation.

These hypotheses are that neuroligin-2 is necessary 1) for attainment of normal pancreatic islet mass and total insulin content 2) for attainment of normal islet size 3) for β-cells to have normal insulin content 4) for normal glucose homeostasis. Pancreases from 6 each of Cre-positive Nlgn2fl/fl and littermate control Nlgn2fl/fl and Ins1-Cre male mice at 8-10 weeks of age will be sectioned for immunofluorescence analysis and another 6 of each for measurement of pancreatic insulin content (normalized to total protein content). To help examine maturation and differentiated state, qPCR is used to compare transcript levels of MafA, urocortin, Glut2 and glucokinase and of constituents of the synaptic-like secretory machinery, including Munc18, syntaxin and snap25, in islets from mutant and control mice (Ostenson et al., 2006, Diabetes 55:435-40; Aguayo-Mazzucato et al., 2011, Diabetologia 54:583-93; Blum et al., 2012, Nat Biotechnol 261-4). Both of the analyses shown in FIG. 28 are repeated with these mice. Glucose homeostasis and islet function are analyzed by glucose tolerance testing, feeding of a high-fat diet and by islet perifusion in parallel with studies of the inducible Nlgn2 knockout mice described below.

What is the Effect of β-Cell-Specific Loss of Neuroligin-2 β-Cell Function on Glucose Homeostasis in Mice?

To avoid effects due to potentially abnormal islet development resulting from the constitutive absence of neuroligin-2 in the model above, islet function and glucose homeostasis is also tested in mice with β-cell specific neuroligin-2 knockout induced in adulthood. The mouse strain Tg (Ins1-CreERT) (MIP1-CreERT) are used. Cre expression in this mouse has no effect on islet morphology or function, however the construct used to express Cre results in growth hormone expression (Tamarina et al., 2014, Islets 6:e2685; Brouwers et al., 2014, Cell Metab 20:979-90). This affects some aspects of islet physiology but not studies using isolated islets or in vivo glucose tolerance tests and is addressed by using MIP1-CreERT littermate controls (Brouwers et al., 2014, Cell Metab 20:979-90; Magnuson et al., 2013, Cell Betab 18:9-20; Oropeza et al., 2015, Diabetes 64:3798-807). Eight week old mice are treated with 200 mg/kg of tamoxifen for five consecutive days (controls and KO mice will be treated in parallel). In work characterizing the MIP1-CreERT mice, this regimen resulted in loss of a foxed gene sequence in 92% of β-cells without an effect of the tamoxifen on subsequent glucose tolerance (Tamarina et al., 2014, Islets 6:e2685). Mice are employed in studies 2 to 3 weeks after the last tamoxifen injection.

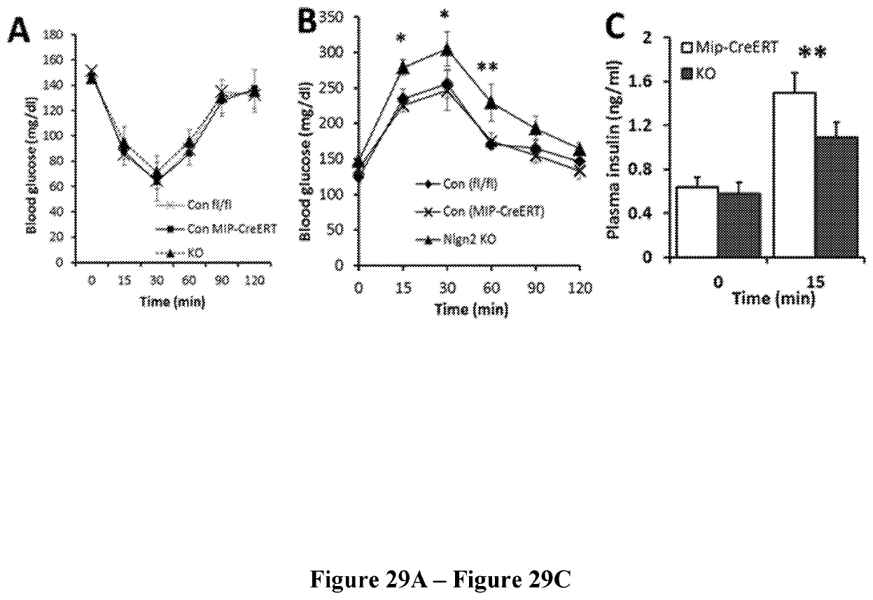

The effect of neuroligin loss on glucose homeostasis is assessed by measurements of plasma glucose, C-peptide and glucagon and by standard glucose tolerance testing (IPGTT) in seven to ten each of male and female KO mice, Cre-negative (control) Nrlg2fl/fl littermates and MIP-CreERT control littermates (see Vertebrate Animals regarding mouse numbers). All mice are on a C57Bl6/J background. The development of obesity and hyperglycemia in this strain as a result of a high-fat diet has been well-characterized, and it is a commonly-employed model of diet-induced diabetes (Surwit et al., 1995, Metabolism 44:645-51; Surwit et al., 1988, Diabetes 37:1163-7). Glucose homeostasis and β-cell function is challenged by feeding the mice a high fat diet for 12 weeks (nine each of male and female Nrlgn2fl/fl:MIP1-CreERT mice and Nrlgn2fl/fl and MIP-CreERT littermate controls) to determine susceptibility to impaired glucose tolerance—as measured by glucose tolerance testing and measurements of fasting insulin and glucose—under diabetogenic conditions (Kozak et al., 2002, Ann N Y Acad Sci 967:80-7; Kossmeisl et al., 2003, Diabetes 52:1958-66). The conditional knockout does not affect weight (not shown) or insulin sensitivity (FIG. 29). While glucose homeostasis resembles that in controls at baseline, glucose tolerance is impaired and plasma insulin 15 min after glucose administration is lower (FIG. 29B and FIG. 29C).

Basal and stimulated insulin secretion by isolated islets are analyzed in standard fashion by static culture and by perifusion. To examine maturation and differentiated state, qPCR is used to compare transcript levels of MafA, urocortin, Glut2 and glucokinase and of constituents of the synaptic-like secretory machinery, including Munc18, syntaxin and snap25, in islets from mutant and control mice. Epac2 participates in GLP-1-mediated promotion of insulin secretion (Holz et al., 2004, Diabetes 53:5-13). To help test whether Epac2 function is impaired in knockout mice, insulin secretory response to GLP-1 is assessed.

Is β-Cell Voltage-Dependent Calcium Channel (VDCC) Function Impaired in Neuroligin-2 Knockout Mice?

By recruiting neurexin to sites of exocytosis (FIG. 18), it is hypothesized that neuroligins also functions to establish normal β-cell VDCC function. This is because alpha-neurexins are essential for VDCC function (Siddiqui et al., 2011, Curr Opin Neuorobiol 21:132-43; Dudanova et al., 2006, J Neurosci 26:10599-613). Enhancing VDCC function is therefore a mechanism whereby extracellular neuroligin interactions—as seen in the coculture model—enhance insulin secretion. Perfusion-based methodologies allowing simultaneous measurement of insulin secretion rate, intracellular calcium concentration and oxygen consumption are used to analyze islets from neuroligin-2 β-cell knockout mice and controls generated as described above using both constitutively-expressing and inducible Cre-expressing strains. Islets are loaded with the ratiometric calcium indicator fura-2AM and then insulin secretion and intracellular calcium concentration are assessed in parallel at baseline (3 mM glucose) and then at high glucose (20 mM). In mice, non-L-type VDCCs may be particularly important for second-phase insulin secretion. To analyze the effect of neuroligin deficiency on non-L-type VDCC function, the intracellular calcium levels, baseline insulin secretion and the magnitude of increase in glucose-stimulated insulin secretion are assessed after increasing glucose concentration from 3 mM to 20 mM in perifusion buffer containing the L-type dihydropyridine channel blocker nimodipine (5 μM). ω-Conotoxin GVIA and ω-agatoxin IVA is substituted in place of nimodipine to block N and P/Q-type channels in order to analyze effects on the L-type channel (the major β-cell VDCC). To test the prediction that non-VDCC-dependent amplification pathways—which are likely less to be dependent on normal excitosome assembly—remain intact despite neuroligin deficiency, knockout and control islets stimulated with 20 mM glucose are further treated with 0.25 mM IBMX, allowing evaluation of whether cAMP-mediated amplification of insulin secretion remains operative.

β-cell specific neuroligin-2 knockdown in mice results in smaller islets, reduced β-cell mass, lower β-cell insulin content, changes in insulin secretion, altered glucose homeostasis and increased susceptibility to diet-induced diabetes, and perhaps impaired calcium-channel function and reduced expression at the mRNA level of markers of functional maturation.

### Example 4: Synaptogenic Synaptic Cleft Proteins that are Present on the β-Cell Surface and that Engage in Trans-Cellular Interactions that Promote β Cell Function

The discovery that neuroligins and neurexins are synaptogenic has been followed by the identification of numerous other synaptogenic proteins (Missler et al., 2012, Cold Spring Harb Perspect Biol 4:a005694; Siddiqui et al., 2011, Curr Opin Neuorobiol 21:132-43). Transmembrane synaptogenic proteins are defined by the ability to induce formation of a pre- or post-synaptic site (a “hemi-synapse”) when expressed on the surface of a cell (or attached to a bead) and then brought into contact with a neuronal process. The picture that is emerging is one of synapse formation and maintenance of function being guided by a network of trans-cellular protein interactions across the synaptic cleft. This is potentially of great significance to the understanding of the β-cell surface. The limited number of transmembrane neuronal proteins that have been identified and characterized on the β-cell surface all influence insulin secretion or (in the case of NCAM) islet architecture. The thus-far identified and characterized β-cell surface neuronal proteins are: NCAM, isoforms of EphA and ephrinA, and neuroligin, neurexins and CADM family members: a total of 6 proteins/protein families (Suckow et al., 2008, Endocrinol 149:6006-17; Kelly et al., 2011, Islets 3:41-7; Konstantinova et al., 2007, Cell 129:359-70).

It is hypothesized herein that a distinct subset of neuronal synaptogenic proteins is expressed on the β-cell surface and that, as in the synapse, these proteins promote maturation and function through trans-cellular interactions. qPCR was used to analyze synaptogenic gene expression in INS-1 cells and rat islets (Table 2).

TABLE 2

| | Islets | INS1 | | Islets | INS1 |
|---|---|---|---|---|---|
| CADM1 | 13.0 | 140.0 | PTPRD | 6.4 | 0.8 |
| CADM2 | 29.9 | 1.1 | PTPRS | 9.7 | 32.3 |
| CADM3 | 8.0 | 13.0 | LAR | 432.0 | 40.7 |
| CADM4 | 71.1 | 33.0 | NGL1 | 9.5 | 8.4 |
| LRRTM1 | 3.5 | 2.3 | NGL2 | 0.4 | 1.2 |
| LRRTM2 | 31.5 | 42.0 | NGL3 | 8.0 | 1.0 |
| LRRTM3 | 5.6 | 2.3 | CLSTN1 | 1.5 | 2.4 |
| LRRTM4 | 15.4 | 23.5 | CLSTN2 | 1.5 | 48.1 |
| Slitrk1 | 0.5 | ND | CLSTN3 | 4.8 | 6.8 |
| Slitrk2 | 2.3 | 0.6 | IL1RAPL1 | 0.1 | ND |
| Slitrk3 | 0.1 | 0.1 | IL1RAcP | 14.2 | 105.0 |
| Slitrk4 | 0.3 | ND | IL1RAcPb | 0.9 | 1.2 |
| Slitrk5 | 0.8 | 1.0 | SALM3 | 43.3 | 41.5 |
| Slitrk6 | 517.0 | 498.0 | SALM5 | 0.6 | 2.0 |

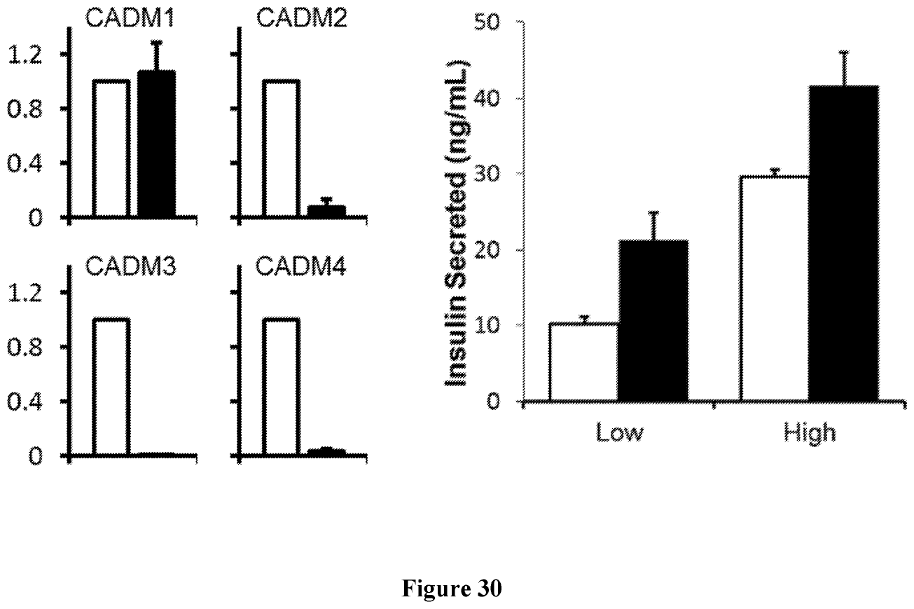

Coculture system was used to investigate trans-cellular interactions between HEK293 cells expressing the synaptogenic protein CADM1 and INS-1 β-cells (FIG. 30) and primary rat islet cells (not shown). Like neuroligin, CADM1 enhances insulin secretion in a trans-cellular manner (FIG. 24). EphA/ephrinA also interact extracellularly to help regulate insulin secretion, further evidence of the value of elucidating the role of synaptogenic proteins.

In addition to the neuroligins, neurexins, and certain Ephs and ephrins, nine other protein families that trigger synaptogenesis through trans-cellular interactions have been identified to date: 1) The CADM (SynCAM) proteins are found on both pre- and post-synaptic membranes (Biederer et al., 2002, Science 277:1525-31). 2) The LRRTM proteins are, like neuroligin, neurexin binding partners (Siddiqui et al., 2013, Neuron 79:680-95). 3) The Slitrk proteins are postsynaptic and bind 4) presynaptic receptor-type protein tyrosine phosphatases (PTPD, PTPS, LAR) to drive synapse formation and maturation (Takahashi et al., 2013, Trends Neurosci 36:522-34; Yim et al., 2013, PNAS 110:4057-62). 5) The netrin G ligands (NGLs) are postsynaptic and second only to the neurexins as the most potent inducers of synaptogenesis (Woo et al., 2009, Nat Neurosci 12:428-37; Siddiqui et al., 2011, Curr Opin Neuorobiol 21:132-43). Their binding partners include the presynaptic receptor-type protein tyrosine phosphatases. 6) The CLSTN (calsyntenin) proteins are postsynaptic binding partners of α-neurexins (Pettem et al., 2013, Neuron 80:113-28). 7) Interleukin-1 receptor accessory protein (IL1RA) variants link the immune system to synapse formation and possibly to β-cell function (Yoshida et al., 2012, J Neurosci 32:2588-600). 8) The SALM proteins are postsynaptic (Mah et al., 2010, J Neurosci 30:5559-68). 9) Cerebellins (Cbln-1 and -2) are also postsynaptic binding partners of neurexins (Matsuda et al., 2011, Eur J Neurosci 33:1447-61).

Which Synaptogenic Proteins are Expressed in Rat and Human Islet β-Cells?

Table 2 shows qPCR results of expression at the mRNA level. Transcripts encoding 14 synaptogenic proteins are present at levels at least 10% of brain levels in INS-1 cells and rat islets, and others are present at lower levels relative to brain. qPCR studies are used to determine whether there is β-cell expression of Cbln and new synaptogenic proteins that may be discovered during the project period. Transcript levels are determined using cDNA derived from FACS-sorted human islet β-cells. Expression at the protein level are analyzed by immunostaining studies of rat and human pancreas sections. Physiologically relevant expression of proteins is identified by gene silencing experiments using INS-1 and rat islet cells. Cells are treated with siRNA and insulin secretion is analyzed. Alteration of insulin secretion as a result of reduced expression will provide evidence of physiologically relevant expression.

Which Synaptogenic Proteins, in Addition to Neuroglin-2, Neurexins, CADMs and Eph/Ephrin, Influence Insulin Secretion and/or Maturation of the Insulin Secretory Machinery?

Synaptogenic proteins expressed at the transcript level in β-cells are analyzed in the coculture system. cDNAs in mammalian expression vectors are transfected into HEK293 cells. Forty eight to 72 hours after transfection, co-cultures are be established by pipetting INS-1 cells or dissociated rat islet cells onto the layer of transfected HEK293-cells. Proteins found to influence insulin secretion in these experiments are also tested in coculture with dissociated human islets. Control HEK293 cells are be mock-transfected or transfected with the neuronal, non-synaptic, transmembrane protein CASPR. There are no differences in insulin secretion between cocultures with non-transfected HEK293 cells, HEK293 cells transfected with empty vector and HEK293 cells transfected with CASPR. In other words, HEK293 transfection with an empty vector or with a control protein alone does not induce functional changes in co-cultured β-cells. Basal and glucose-stimulated insulin secretion from β-cells are compared in control cocultures and β-cells co-cultured with HEK293 cells expressing the protein of interest. Promotion of submembrane secretory complex formation is assessed by determining the punctateness of syntaxin staining.

### Example 5: Delivery of Synapse-Inducing Proteins, Synapse-Inducing Protein Fragments, Synapse-Inducing Protein-Derived Peptides and Peptidomimetics

Synapse-inducing proteins, synapse-inducing protein fragments, synapse-inducing protein-derived peptides and peptidomimetics can be used to treat diabetes or to generate insulin secreting cells. For Example, as described in experimental example 1, recombinant NL-2 and NL-2-derived peptides or peptidomimetics can be used treat diabetes and/or aid in the generation of insulin-secreting cells from stem cells are able to aid in the generation of insulin-secreting cells from stem cells.

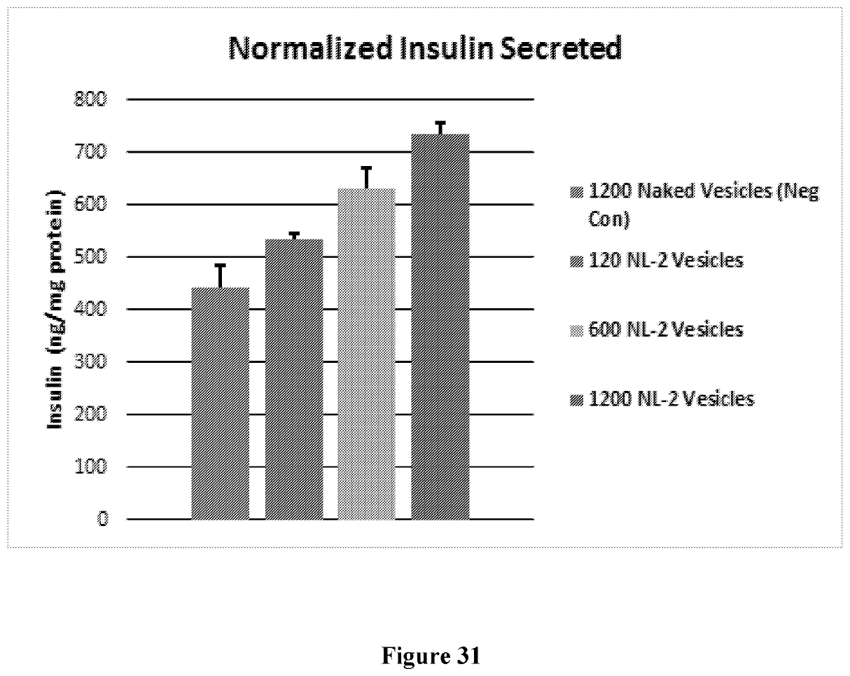

Delivery of these proteins, protein fragments, and protein-derived peptides and peptidomimetics can be achieved by conjugating the peptides to the surface of nanoparticles, cross-linking the proteins into protein complexes or delivery through lipid nanoparticles or liposomes. For example, FIGS. 3 and 12 demonstrates that a crosslinked NL-2-derived peptide and a NL-2-derived peptide conjugated to a nanoparticle, respectively, each increases insulin secretion. Further, FIG. 31 demonstrates increased insulin secretion by beta cells treated with increasing amounts of lipid particles carrying recombinant NL-2. It is noted that a near doubling of insulin secretion is observed and the upper plateau of the dose-response curve has not yet been reached. It is further noted that the vesicles used in this experiment have been stored for over a week at room temperature, an indication of good stability.

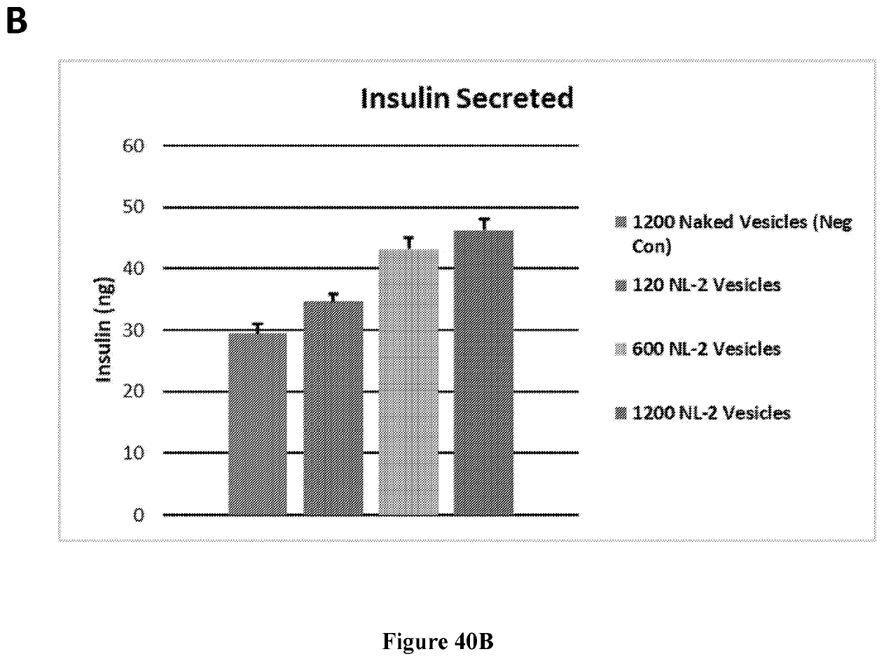

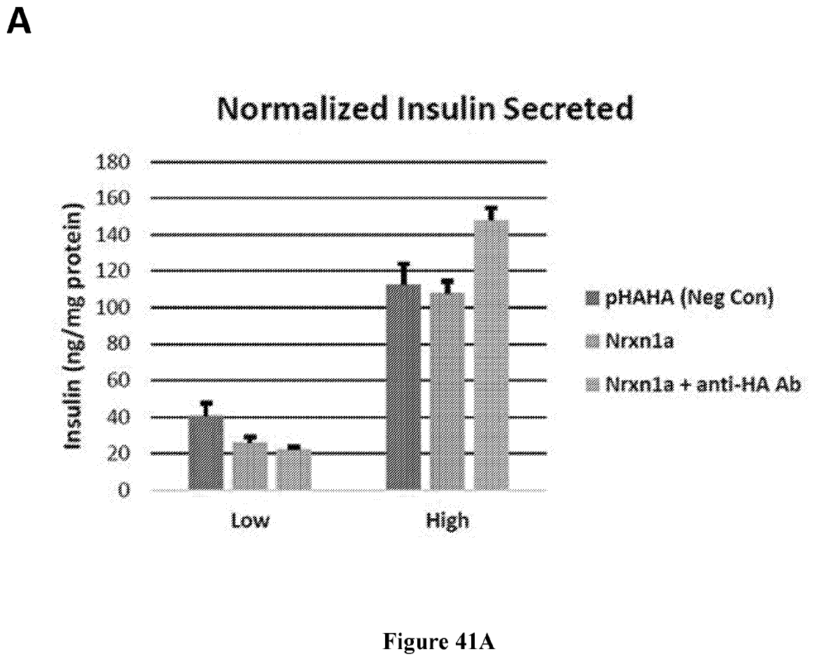

To further demonstrate that the recombinant neuroligin-2 extracellular domain can improve pancreatic beta cell function, the protein was attached to artificial lipid particles (FIG. 40). Control particles and increasing amounts of therapeutic vesicles (with the protein attached) were incubated in culture with beta cells. Insulin secretion was assessed 24 hours later. As can be seen, insulin secretion increased in a dose-dependent fashion. FIG. 40A depicts insulin normalized to cellular insulin content. FIG. 40B depicts absolute insulin secretion. These results demonstrate, with a different therapeutic particle, that clustered neuroligin-2 can increase insulin secretion. This extends the data demonstrated in Example 1 to demonstrate that clustered neuroligin-2 conjugated to more conventional nanoparticles can increase insulin secretion.

### Example 6: Neuroligand-2 Binds and Clusters Nrxn1a

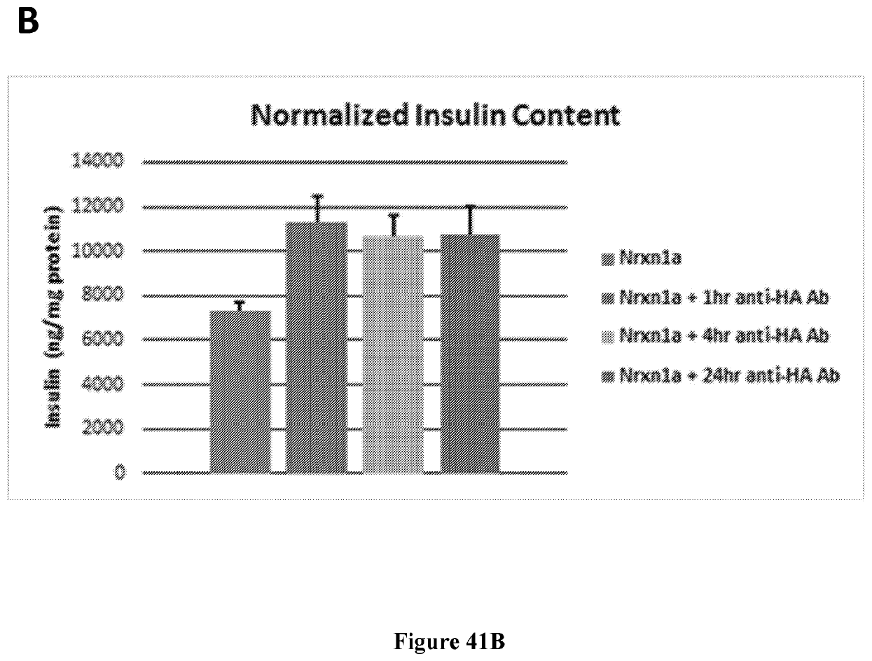

The extracellular domain of neuroligin-2 binds and clusters the extracellular domain of the protein neurexin-1 (Nrxn1a). It is hypothesized herein that recombinant neuroligin-2 and neuroligin-2-derived peptides exert their beneficial effects by binding and clustering Nrxn1a. Indeed, binding and clustering Nrxn1a with other agents may be another approach to activating the neuroligin-neurexin pathway. To test this, pancreatic beta cells were transfected so that they would produce Nrxn1a modified with an extracellular epitope tag. To bind and cluster neurexin-1, an antibody was introduced that binds the epitope tag into the beta cell cultures (FIG. 41). FIG. 41A demonstrates that at low glucose (left), using an antibody to cluster neurexin (gray column) did not increase insulin secretion (blue column is non-transfected negative control). At high glucose levels (right three columns), the antibody increased insulin secretion (orange vs gray column). FIG. 41B demonstrates that incubating with the antibody also increased the insulin content of the beta cells (blue column vs the other three). Increased insulin content was seen within 1 hour (red column) of antibody incubation.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising an agent that increases a β-cell surface protein activity.

2. The composition of claim 1, wherein the β-cell surface protein is selected from the group consisting of CADM1, CADM2, CADM3, CADM4, LRRTM1, LRRTM2, LRRTM3, LRRTM4, NEUREXIN-1, NEUREXIN-2, NEUREXIN-3, Slitrk1, Slitrk2, Slitrk3, Slitrk4, Slitrk5, Slitrk6, PTPRD, PTPRS, LAR, NL-1, NL-2, NL-3, CLSTN1, CLSTN2, CLSTN3, IL1RAPL1, IL1RAcP, IL1RAcPb, SALM3, and SALM5.

3. The composition of claim 2, wherein the β-cell surface protein is NL-2.

4. The composition of claim 1, wherein the agent comprises a protein, wherein the protein binds neurexin isoforms and mimics neuroligin activity.

5. The composition of claim 1, wherein the agent comprises an isolated peptide.

6. The composition of claim 5, wherein the isolated peptide comprises a β-cell surface protein-derived peptide or a NL-2-derived peptide.

7. (canceled)

8. The composition of claim 5, wherein the isolated peptide comprises an amino acid sequence selected from SEQ ID NOs: 1-3.

9. The composition of claim 8, wherein the isolated peptide comprises a dimer of two peptides comprising at least one amino acid sequence selected from SEQ ID NOs: 1-3.

10. The composition of claim 9, wherein the dimer is conjugated to PEG-2000.

11. The composition of claim 5, wherein the isolated peptide is conjugated to the surface of a delivery vehicle.

12. (canceled)

13. The composition of claim 11, wherein the delivery vehicle is a nanoparticle and the nanoparticle comprises a maghemite.

14. The composition of claim 13, wherein the nanoparticle further comprises an Ytterbium.

15. The composition of claim 12, wherein the nanoparticle is a Yb(III) cation doped-maghemite nanoparticle.

16. A cell engineered to secrete insulin, wherein the cell expresses a recombinant β-cell surface protein or a β-cell protein-derived peptide.

17. The cell of claim 16, wherein the β-cell surface protein is selected from the group consisting of CADM1, CADM2, CADM3, CADM4, LRRTM1, LRRTM2, LRRTM3, LRRTM4, NEUREXIN-1, NEUREXIN-2, NEUREXIN-3, Slitrk1, Slitrk2, Slitrk3, Slitrk4, Slitrk5, Slitrk6, PTPRD, PTPRS, LAR, NL-1, NL-2, NL-3, CLSTN1, CLSTN2, CLSTN3, IL1RAPL1, IL1RAcP, IL1RAcPb, NGL1, NGL2, NGL3, SALM3, and SALM5.

18. The cell of claim 16, wherein the cell expresses NL-2 or a NL-2 derived peptide.

19. The cell of claim 17, wherein the cell expresses a peptide having an amino acid sequence selected from the group consisting of SEQ ID NOs: 1-3.

20. A method for treating or preventing a condition associated with reduced insulin secretion in a subject in need thereof, the method comprising administering to the subject an effective amount of the composition of claim 1.

21.-30. (canceled)

31. The method of claim 20, wherein the condition is diabetes.

32. A method for treating or preventing a condition associated with reduced insulin secretion in a subject in need thereof, the method comprising:
differentiating a stem cell into a mature β-cell by culturing the stem cell in the presence of a composition comprising an agent that increases a β-cell surface protein activity, thereby producing a cluster of mature β-cells; and
transplanting the cluster of mature β-cells to the subject.
